(19) United States
(12) Patent Application Publication
(10) Pub. No.: US 2022/0242809 A1
(43) Pub. Date: Aug. 4, 2022

(54) METHODS FOR ISOLATING ESSENTIAL OILS, PHYTOCHEMICALS AND PHARMACEUTICALLY ACTIVE COMPONENTS FROM BIOMASS AND RECONSTITUTING SYNTHETIC FORMULATIONS COMPRISING SAME

(71) Applicant: Atlas Hemp Company LLC., Seattle, WA (US)

(72) Inventor: Robert H. MAHDAVI, Seattle, WA (US)

(21) Appl. No.: 17/626,707

(22) PCT Filed: Jul. 10, 2020
(86) PCT No.: PCT/US2020/041705

§ 371 Date: Jan. 12, 2022

Related U.S. Application Data
(63) Continuation of application No. 62/873,597, filed on Jul. 12, 2019.

(51) Int. Cl. C07C 37/50; C07C 37/70

(52) U.S. Cl. CPC C07C 37/50; C07C 37/70

(57) ABSTRACT

In alternative embodiments, provided are methods for extracting essential oils, phytochemicals and pharmaceutically active components such as terpenes, phytochemicals and pharmaceutically active components from biomass or any plant materials. In alternative embodiments, provided are methods for making synthetic formulations comprising biomass-derived plant materials using essential oils, phytochemicals and pharmaceutically active components isolated by methods as provided herein. In alternative embodiments, provided are synthetic biomass-derived formulations and plant materials using essential oils, phytochemicals and pharmaceutically active components isolated by methods as provided herein.

### RELATED APPLICATIONS

This patent Convention Treaty (PCT) International Application claims the benefit of priority to U.S. Provisional Application No. 62/873,597 filed Jul. 12, 2019. The aforementioned application is expressly incorporated herein by reference in its entirety and for all purposes.

### TECHNICAL FIELD

This invention generally relates to organic chemistry and the extraction of essential oils, phytochemicals and pharmaceutically active components from biomass or any plant materials. In alternative embodiments, provided are methods for extracting essential oils, phytochemicals and pharmaceutically active components such as terpenes, phytochemicals and pharmaceutically active components from biomass or any plant materials. In alternative embodiments, provided are methods for making synthetic formulations comprising biomass-derived plant materials using essential oils, phytochemicals and pharmaceutically active components isolated by methods as provided herein. In alternative embodiments, provided are synthetic biomass-derived formulations and plant materials using essential oils, phytochemicals and pharmaceutically active components isolated by methods as provided herein.

### BACKGROUND

Essential oils, phytochemicals and pharmaceutically active components from biomass or plant materials are a growing industry with increasing consumer demand. The exits a need for efficient and cost effective ways to analyze and isolate essential oils, phytochemicals and pharmaceutically active components from biomass and plants, and make plant-derived formulations with desired properties.

Several classes of phytochemicals are produced by the Cannabis Sativa L. plant including cannabinoids, terpenoids, flavonoids, cuticle waxes and other hydrocarbons. The quantitative distribution of these materials varies from one strain to another. The specific combination of compounds in a given strain work synergistically, or as an entourage, to create a characteristic flavor, odor, or therapeutic effect.

Terpenes are a class of chemical compounds consisting of combinations of isoprene units. The conjugated double bonds in the isoprene subunit are what impart the fragrance and bioactivity to terpenes. The terpenes found in Cannabis Sativa L. are primarily monoterpenes, consisting of two isoprene units, and sesquiterpenes, consisting of three isoprene units. Functional groups with oxygen may also be present in which case the compounds are properly referred to as terpenoids, rather than terpenes.

Much attention has been placed on the concentrations of various cannabinoids in Cannabis Sativa L. because of the psychoactive effect of Δ9-tetrahydrocannabinol (Δ9-THC), or potential medical benefits in the case of cannabidiol (CBD) and other cannabinoids. However, a larger part of the identifying characteristics of specific strains may be attributed to the terpenoid component of the chemical profiles. Cannabinoids do not have any odor, but terpenes are intensely fragrant even at low concentrations because of their higher volatility and chemical structure. Many of the terpenes are bioactive, with sedative or energizing effect, or therapeutic value including anti-cancer activity, control of epileptic seizures and other efficacies. Hundreds of different terpenes are typically present in a plant and the distribution of these terpenes is correlated to the genetics of the specific strain. With so many possible combinations, the fragrances and bioactivity of the individual terpenes combine synergistically to create a character unique to a given strain.

### SUMMARY

In alternative embodiments, provided are synthetic or non-natural formulations comprising a non-natural mix of bio-mass or plant-derived terpenes or terpenoids,

wherein optionally the terpenes or terpenoids comprise at least one, 2, 3, 4, 5, 6, 7, 8, 9, 10, 11, 12, 13, 14, 15, 16, 17, 18, 19 or 20 or more terpene or terpenoid species listed in Table 1, or at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thuj anol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Farnesene, (6E)-; beta-Ocimene; beta-Pinene; beta-Thujene; Borneol; Camphene; Camphor; Cannabidiol; Cannabigerolic Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isoborneol, (−)-; Isopulegol; Limonene; Linalool; Menthol; Myrcene; Nerol; Nerolidol; p-Cymene; Phytol; Pulegone; Sabinene; Sabinene Hydrate; Terpineol; Terpinolene; Valencene; (−)-Terpinen-4-ol, and (−)-Terpinen-4-ol,

and optionally the synthetic or non-natural formulation comprises all but one, 2, 3, 4, 5, 6, 7, 8, 9, 10, 11, 12, 13, 14, 15, 16, 17, 18, 19 or 20 or more of the compounds listed in Table 1, or at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Farnesene, (6E)-; beta-Ocimene; beta-Pinene; beta-Thujene; Borneol; Camphene; Camphor; Cannabidiol; Cannabigerolic Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isoborneol, (−)-; Isopulegol; Limonene; Linalool; Menthol; Myrcene; Nerol; Nerolidol; p-Cymene; Phytol; Pulegone; Sabinene; Sabinene Hydrate; Terpineol; Terpinolene; Valencene; (−)-Terpinen-4-ol, and (−)-Terpinen-4-ol,

and optionally the only terpene or terpenoid species in the synthetic or non-natural formulation are the at least one, 2, 3, 4, 5, 6, 7, 8, 9, 10, 11, 12, 13, 14, 15, 16, 17, 18, 19 or 20 or more terpene or terpenoid species listed in Table 1, or at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Farnesene, (6E)-; beta-Ocimene; beta-Pinene; beta-Thujene; Borneol; Camphene; Camphor; Cannabidiol; Cannabigerolic Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isoborneol, (−)-; Isopulegol; Limonene; Linalool; Menthol; Myrcene; Nerol; Nerolidol; p-Cymene; Phytol; Pulegone; Sabinene; Sabinene Hydrate; Terpineol; Terpinolene; Valencene; (−)-Terpinen-4-ol, and (−)-Terpinen-4-ol,

and optionally the synthetic or non-natural formulation, other than a solvent, thinner, diluent, dilutant or excipient, consists of only at least one, 2, 3, 4, 5, 6, 7, 8, 9, 10, 11, 12, 13, 14, 15, 16, 17, 18, 19 or 20 or more terpene or terpenoid species listed in Table 1, or at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Farnesene, (6E)-; beta-Ocimene; beta-Pinene; beta-Thujene; Borneol; Camphene; Camphor; Cannabidiol; Cannabigerolic Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isoborneol, (−)-; Isopulegol; Limonene; Linalool; Menthol; Myrcene; Nerol; Nerolidol; p-Cymene; Phytol; Pulegone; Sabinene; Sabinene Hydrate; Terpineol; Terpinolene; Valencene; (−)-Terpinen-4-ol, and (−)-Terpinen-4-ol,

and optionally the synthetic or non-natural formulation comprises a formulation of any one of Formula #1 to #164, as described herein.

In alternative embodiments of synthetic or non-natural formulations as provided herein:

at least one, 2, 3, 4, 5, 6, 7, 8, 9, 10, 11, 12, 13, 14, 15, 16, 17, 18, 19 or 20 or more of the terpene or terpenoid species is a synthetic terpene or terpenoid, or

the synthetic or non-natural formulation further comprises a non-terpene or non-terpenoid plant-derived phytochemical,

wherein optionally the non-terpene or non-terpenoid plant-derived phytochemical comprises a phytocannabinoid, a flavonoid, a cuticle wax or a plant-derived hydrocarbon,

wherein optionally the non-terpene or non-terpenoid plant-derived phytochemical, or the phytocannabinoid, comprises: tetrahydrocannabinol (THC), tetrahydrocannabivarin (THCV), cannabichromene (CBC), cannabichromanon (CBCN), cannabidiol (CBD), cannabielsoin (CBE), cannabidivarin (CBDV), cannbifuran (CBF), cannabicyclol (CBL), Cannabinol (CBN), Cannabinodiol (CBND), cannabitriol (CBT), cannabivarin (CBV), an isocanabinoid, cannabidiolic acid (CBDA), Δ9-tetrahydrocannabinol (Δ9-THC) or Δ8-tetrahydrocannabinol (Δ8-THC),

wherein optionally the non-terpene or non-terpenoid plant-derived phytochemical, or the phytocannabinoid, comprises: cannabigerolic Acid (CBGA), Cannabigerolic Acid, monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBDVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinol (THC), Tetrahydrocannabinolic acid C₄ (THCA-C₄), Tetrahydrocannbinol C₄ (THC-C₄), Tetrahydrocannabivarinic acid (THCVA), Tetrahydrocannabivarin (THCV), Tetrahydrocannabiorcolic acid (THCA-C1), Tetrahydrocannabiorcol (THC-C₁), Delta-7-cis-iso-tetrahydrocannabivarin, Δ⁸-tetrahydrocannabinolic acid (A8-THCA), Δ⁸˜tetrahydrocannabinol (Δ⁸-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C₄ (CBN-C₄), Cannabivarin (CBV), Cannabino-C₂ (CBN-C2), Cannabiorcol (CBN-C₁), Cannabinodiol (CBND), Cannabinodivarin (CBDV), Cannabitriol (CBT), 10-Ethoxy-9-hydroxy-Δ^{6a}-tetrahydrocannabinoi, 8, 9-Dihydroxy-Δ^{6a(10a)}-tetrahydrocannabinol (8,9-Di-OH-CBT-05), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ^{6a(10a)}-tetrahydrocannabinol (OTHC), Δ⁹-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Isocanabinoids, Epigallocatechin gallate, Dodeca-2E, 4E, 8Z, 10Z-tetraenoic acid isobutylamide, or Dodeca-2E,4E-dienoic acid isobutylamide,

wherein optionally the terpene, terpenoid or flavonoid is or comprises d-limonene linalool, 1,8-cineole (eucalyptol), α-pinene, terpineol-4-ol, p-cymene, borneol, Δ-3-carene, β-sitosterol, β-myrcene, β-caryophyllene, cannflavin A, apigenin, quercetin or pulegone or any combination thereof,

wherein optionally the terpene, terpenoid or flavonoid is or comprises: alpha-bisabolol; borneol; camphene; camphor; delta-3-carene; beta-caryophyllene; caryophyllene oxide; alpha-cedrene; beta-eudesmol; fenchyl alcohol; geraniol; guaiol; alpha-humulene; isoborneol; limonene; linalool; menthol; myrcene; nerol; ocimene; trans-ocimene; alpha-phellandrene; alpha-pinene; beta-pinene; sabinene; alpha-terpinene; alpha-terpineol; terpinolene; alpha-guaiene; elemene; farnesene; germacrene B; guaia-1(10), 11-diene; trans-2-pinanol; selina-3,7(11)-diene; eudesm-7(11)-en-4-ol; and/or valencene,

wherein optionally the terpene, terpenoid or flavonoid is or comprises: 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (β-Bisabolol), Borneol; Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),1 1-diene, Guaiacol, Guaiene (α-Guaiene), Gurjunene (α-Gurjunene), Herniarin, Hexanaldehyde, Hexanoic Acid, Humulene (α-Humulene) (β-Humulene), lonol (3-oxo-α-ionol) (β-IoηoI), lonone (α-lonone) (β-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, AIM Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, p-cymene, pentyl butyrate, phellandrene, phenylacetaldehyde, phenylethanethiol, Phenylacetic Acid, Phytol, Pinene, β-Pinene, propanethiol, Pristimerin, Pulegone, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone or Vanillin, and/or

wherein optionally the terpene, terpenoid or flavonoid is or comprises: a phenolic acid, a stilbenoid, a dihydroflavonol, an anthocyanin, an anthocyanidin, a polyphenol, a tannin, a flavone, flavan-3-ol, flavan-4-ol, flavan-3,4-diol flavonol, a stilbenoid, a phytochemicals, an antioxidant, a homoisoflavonoid, a phenylpropanoid, a phloroglucinol coumarin, a phenolic acid, a naphthodianthrone, a steroid glycoside, a bioflavonoid, an isoflavonoid, a neoflavonoid, adenosine, Adhyperforin, amentoflavone, Anandamide, Apigenin, Cannaflavin B, Catechin (C), Catechin 3-gallate (Cg), Chlorogenic acid, cichoric acid, caftaric acid, Daidzein, Delphinidin, Eleutherosides, epicatechin 3-gallate (ECg), Epicatechins, Epicatechin, epigallocatechin, myricetin, Oxalic acid, Pelargonidin, Tannin, Theaflavin-3-gallate, Theanine, Theobromine, Theophylline, Tryptophan, Tyramine, Xanthine, Caffeine, Cannaflavin A, Cannaflavin B, Catechin (C), Catechin 3-gallate (Cg), Epicatechin 3-gallate (ECg), Epicatechins (Epicatechin (EC)), epigallocatechin, Epigallocatechin (EGC), Epigallocatechin 3-gallate (EGCg), Gallocatechin (GC), Gallocatechin 3-gallate (GCg)), Gamma amino butyric acid, Genistein, Ginkgo biloba, Ginsenosides, Quercetin, Quercitrin or Rutin.

In alternative embodiments synthetic or non-natural formulation as provided herein further comprise:
a synthetic compound or a non-plant derived compound, wherein optionally the synthetic compound comprises a synthetic: cannabinoid, a terpenoid, a flavonoid, a cuticle wax or a synthetic version of a plant-derived hydrocarbon, or a synthetic d-limonene linalool, 1,8-cineole (eucalyptol), α-pinene, terpineol-4-ol, p-cymene, borneol, Δ-3-carene, β-sitosterol, β-myrcene, β-caryophyllene, cannflavin A, apigenin, quercetin or pulegone, or a synthetic version of a compound as listed in Table 1;
or are formulated with, a solvent, thinner, or a diluent (or dilutant), and optionally the solvent, thinner, or a diluent comprises squalane, squalene, 1,3-propanediol, acetone, acetonitrile, an alcohol, triacetin, dimethyl sulfoxide, dimethylformamide, phenol, propylene glycol, dipropylene glycol, 1,3-propanediol, isophytol, and/or phytol, and optionally the alcohol is or comprises a benzyl alcohol, or ethanol, butanol, 2-ethylhexanol, isobutanol, isopropanol, methanol, propanol or propylene glycol;
or are formulated with, a stabilizer, anti-oxidant or a compound to prevent crystallization, wherein optionally the stabilizer, anti-oxidant or a compound to prevent crystallization comprises triethyl citrate.

In alternative embodiments of synthetic formulations as provided herein: comprise or further comprise a non-plant derived compound or synthetic compound,

and optionally the non-plant derived compound or synthetic compound comprises an organic compound having a odor detectable to a human, wherein optionally the organic compound having a odor detectable to a human comprise or is: 3-methyl-2-butene-1-thiol; hexanoic acid hexyl ester; 2-heptanone, octanal or cis-4-decenal,

and optionally the non-plant derived compound or synthetic compound comprises an organic compound having a taste detectable to a human, wherein optionally the organic compound having a taste detectable to a human comprise or is: 4-hydroxy-2,5-dimethyl-3(2H)-furanone (strawberry), ethyl butyrate (apple, fruity), isoamyl acetate (banana), propyl hexanoate (pineapple, fruity), allyl hexanoate (pineapple, fruity), valencene (orange, fresh fruity), methyl anthranilate (also known as methyl 2-aminobenzoate) (grape), methyl butyrate (fruity, apple, pineapple), benzyl acetate (fruity, strawberry), p-mentha-8-thiol-3-one (grapefruit), (1S,4S)-trans-p-menthan-8-thiol-3-one acetate (black currant, exotic), or (1R,4S)-cis-p-menthan-8-thiol-3-one acetate (fruity, sweet),

and optionally the non-plant derived compound or synthetic comprises a surfactant, and optionally the surfactant comprises or is polysorbate 20, a TWEEN, a stearate, ammonium lauryl sulfate, sodium lauryl sulfate, perfluorooctanesulfonate (PFOS), dioctyl sodium sulfosuccinate, a siloxane, cetrimonium bromide (CTAB), cetylpyridinium chloride (CPC), benzalkonium chloride (BAC), benzethonium chloride (BZT), dimethyldioctadecylammonium chloride, octaethylene glycol monododecyl ether, pentaethylene glycol monododecyl ether and dioctadecyldimethylammonium bromide (DODAB); or the surfactants is an anionic, cationic, nonionic or zwitterionic surfactant.

In alternative embodiments of synthetic or non-natural formulations as provided herein comprise or further comprise: cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C₁), Tetrahydrocannabinolic acid A (THCA-A), tetrahydrocannabinolic acid B (THCA-B), Tetrahydrocannabinol (THC), Tetrahydrocannabinolic acid C₄ (THCA-C₄), Tetrahydrocannbinol C₄ (THC-C₄), Tetrahydrocannabivarinic acid (THCVA), Tetrahydrocannabivarin (THCV), Tetrahydrocannabiorcolic acid (THCA-C₁), Tetrahydrocannabiorcol (THC-C₁), Delta-7-cis-iso-tetrahydrocannabivarin, Δ⁸-tetrahydrocannabinolic acid (Δ⁸-THCA), Δ⁸-tetrahydrocannabinol (Δ⁸-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C₄ (CBN-C₄), Cannabivarin (CBV), Cannabino-C₂ (CBN-C₂), Cannabiorcol (CBN-C₁), Cannabinodiol (CBND), Cannabinodivarin (CBDV), Cannabitriol (CBT), 10-Ethoxy-9-hydroxy-Δ^{6a}-tetrahydrocannabinol, 8,9-Dihydroxy˜Δ^{6a(10a)}˜tetrahydrocannabinol (8,9˜Di-OH-CBT-Cs), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oχo-Δ^{6a(10a)} t-etrahydrocannabinol (OTHC), Δ⁸-c/s˜tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), an isocanabinoid or epigallocatechin gallate.

In alternative embodiments of synthetic or non-natural formulations as provided herein further comprise a mineral, wherein optionally the mineral comprises or is lithium.

In alternative embodiments of synthetic or non-natural formulations as provided herein: the synthetic or non-natural formulation comprises:

(a) one, two, three, 4, 5, 6, 7, 8, 9, 10 or all of the following terepenes: 3-Carene; 3,7(11)-Eudesmadiene; 4-Carvomenthenol; 4-Thuj anol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Farnesene, (6E)-; beta-Ocimene; beta-Pinene; beta-Thujene; Borneol; Camphene; Camphor; Cannabidiol; Cannabigerolic Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isoborneol, (−)-; Isopulegol; Limonene; Linalool; Menthol; Myrcene; Nerol; Nerolidol; p-Cymene; Phytol; Pulegone; Sabinene; Sabinene Hydrate; Terpineol; Terpinolene; Valencene; (−)-Terpinen-4-ol, and (−)-Terpinen-4-ol;

(b) the synthetic or non-natural formulation of (a), wherein the only terpenes in the synthetic formulation are one, two, three, 4, 5, 6, 7, 8, 9, 10 or all of the terpenes listed in (a); or

(c) the synthetic or non-natural formulation of (a), wherein the terpenes are present in approximately equal amounts.

In alternative embodiments provided are products of manufacture comprising or having contained therein a synthetic or non-natural formulation as provided herein, wherein optionally the product of manufacture is a medical device, a patch, a implant or a dispenser (optionally an aerosol dispenser or a nebulizer).

In alternative embodiments provided are pharmaceutical or nutraceutical formations comprising a synthetic or non-natural formulation as provided herein, wherein optionally the pharmaceutical or nutraceutical formulation is manufactured or formulated for oral, sublingual, buccal, aerosol or topical administration, wherein optionally the pharmaceutical or nutraceutical formulation is manufactured or formulated as a liquid, a tincture, an aerosol, a gel, a geltab, a capsule, a tablet, a mist, or a powder, and, wherein optionally the pharmaceutical or nutraceutical formulation further comprises a pharmaceutically acceptable excipient, or a sweetener, wherein optionally the sweetener is a Stevia extract.

In alternative embodiments provided are methods for treating, preventing or ameliorating a cancer or an epileptic seizure in an individual in need thereof, or for generating a sedative or energizing effect in an individual in need thereof, comprising administering a pharmaceutical or nutraceutical formation as provided herein.

In alternative embodiments provided are uses of a product of manufacture as provided herein for treating, preventing or ameliorating a cancer or an epileptic seizure in an individual in need thereof, or for generating a sedative or energizing effect in an individual in need thereof, wherein optionally the product of manufacture comprises a synthetic or non-natural formulation as provided herein, or a pharmaceutical or nutraceutical formation as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1, FIG. 2 and FIG. 3 are illustrations of GCMS chromatograms of the terpene extracts from three different strains of Cannabis Sativa L. with the most abundant terpenes identified; Table 1 shows the concentrations derived from this chromatographic data for 49 terpenes measured for each of the three strains.

FIG. 4 graphically shows the abundance of additional terpenes typically present in Cannabis Sativa L; the upper plot (graph) is scaled to the most abundant compounds; the lower plot (graph) is the same chromatogram expanded to show the many smaller peaks.

FIG. 5 graphically shows the HPLC chromatograms of 17 cannabinoids as listed in Table 1, below.

Like reference symbols in the various drawings indicate like elements.

### DETAILED DESCRIPTION

In alternative embodiments, provided are methods for extracting and/or purifying phytochemicals essential oils, and extracting and/or purifying phytochemicals and pharmaceutically active components such as terpenes, terpenoids, phytochemicals and pharmaceutically active components from a biomass or any plant materials. In alternative embodiments, provided are methods for making synthetic formulations and biomass-derived plant materials using essential oils, phytochemicals and pharmaceutically active components extracted, purified and/or isolated using methods as provided herein. In alternative embodiments, provided are synthetic biomass-derived formulations, for example, nutraceuticals or pharmaceuticals, using essential oils, phytochemicals and/or pharmaceutically active components of plant materials as extracted, purified and/or isolated by methods as provided herein.

In alternative embodiments, provided are synthetic biomass-derived formulations and plant materials having new or enhanced properties, for example, fragrances or flavors, or bioactivities, such as having a therapeutic sedative or an energizing effect, or a therapeutic value including anti-cancer activity or for the control of epileptic seizures. In alternative embodiments, these new or enhanced properties are achieved by formulating novel combinations of individual terpenes or terpenoids extracted, purified and/or isolated from plant materials, including terpenes or terpenoids or other plant materials that are synthetically manufactured. In alternative embodiments, the extracted, isolated or synthetic terpenes or terpenoids are combined with other natural or synthetic compounds such as cannabinoids, flavonoids, cuticle waxes and other hydrocarbons to create a new formulation having a desired property. In alternative embodiments, the isolated or synthetic terpenes or terpenoids are combined with other natural or synthetic compounds to synergistically create a character unique to a given strain.

In alternative embodiments, provided are methods for the chemical characterization and isolation of individual chemical components of a biomass such as a plant; for example for the chemical characterization of an inflorescence of a biomass such as a plant; for example, for isolation of individual chemical components of the inflorescence of different strains of Cannabis, such as Cannabis Sativa L., and the reformulation of these chemical profiles using the isolated compounds to make synthetic formulations or non-natural mixtures of compounds.

The specific combination of phytochemicals (for example, terpenes, terpenoids, flavonoids, waxes and any pharmaceutically active compound contained in a plant) in a given strain of plant, e.g., as in different strains of Cannabis, such as Cannabis Sativa L., work together and synergistically to create the plant's characteristic appearance, flavor, odor, taste and/or potential therapeutic effect. Provided herein are synthetic (or non-natural) formulations comprising non-natural mixtures of these phytochemicals, wherein the synthetic reformulations can comprise phytochemicals from only one plant strain, or from a mix of similar or different plant strains, or from a mix of different plants. In alternative embodiments, the phytochemical reformulations can further comprise non-plant chemicals, including synthetic, microbial (e.g., bacterial) or animal-based chemicals. The final compositions of phytochemical reformulations as provided herein are designed to impart a unique, non-natural property or properties to the phytochemical reformulation; for example, a synthetic reformulation of Cannabis that has no or little psychoactive effect, but retains other properties, for example, a new or altered therapeutic effect, or a specific or unique flavor, feel or odor. The specific combination of phytochemicals used in synthetic reformulations as provided herein include for example, cannabinoids, terpenes, terpenoids, flavonoids, cuticle waxes and other hydrocarbons.

In alternative embodiments, terpenes or terpenoids isolated by methods as provided herein, or used in compositions as provided herein (including for example nutraceuticals or pharmaceuticals as provided herein), or used on methods as provided herein, comprise:

Hemiterpenes, including 2-methyl-1,3-butadiene, hemialboside, and hymenoside;

Monoterpenes, including pinene, alpha-pinene, beta-pinene, cis-pinane, trans-pinane, cis-pinanol, trans-pinanol, limonene; linalool; myrcene; eucalyptol; α-phellandrene; β-phellandrene; α-ocimene; β-ocimene, cis-ocimene, ocimene, delta-3-carene; fenchol; sabinene, borneol, isoborneol, camphene, camphor, phellandrene, α-phellandrene, α-terpinene, geraniol, linalool, nerol, menthol, myrcene, terpinolene, α-terpinolene, β-terpinolene, γ-terpinolene, delta-terpinolene, α-terpineol, and trans-2-pinanol;

Sesquiterpenes, including caryophyllene, caryophyllene oxide, humulene, α-humulene, α-bisabolene; β-bisabolene; santalol; selinene; nerolidol, bisabolol; α-cedrene, β-cedrene, β-eudesmol, eudesm-7(11)-en-4-ol, selina-3,7(11)-diene, guaiol, valencene, α-guaiene, beta-guaiene, delta-guaiene, guaiene, farnesene, α-farnesene, β-farnesene, elemene, α-elemene, β-elemene, gamma-elemene, delta-elemene, germacrene, germacrene A, germacrene B, germacrene C, germacrene D, and germacrene E;

Diterpenes, including oridonin, phytol, and isophytol;

Triterpenes, including ursolic acid, oleanolic acid; and/or

1.5 ene compounds, including guaia-1(10),11-diene, and 1.5 ene. Guaia-1(10),11-diene.

Provided is a chemical characterization of the inflorescence of specific strains of Cannabis sativa L. and the reformulation of these chemical profiles using isolated compounds from a variety of plant derived sources to make new, synthetic formulations. Over 100 different strains of Cannabis sativa L. with validated genetics are grown from seed under controlled consistent environmental conditions. The complete terpene and/or terpenoid components from biomass of each of these strains are quantitatively extracted using several techniques including freeze-dried extraction, super-critical CO₂ extraction, and vacuum-oven extraction with optimized cold-trap and short-path distillation. These extracted materials are analyzed by gas chromatography-mass spectrometry with single ion monitoring for over 50 different terpenes for which standards are available. This analytical data is used to create specific terpene/terpenoid profiles for each of the strains. Recipes derived from these profiles are then used to create strain-specific formulations using isolated terpenes from other sources. Novel, synthetic terpene or terpenoid formulations are made to replicate specific Cannabis strains, for example, to replicate the phytochemical makeup of Cannabis sativa, wherein the new synthetic strain has no or substantially less psychoactive effect from Δ9-THC and/or other cannabinoids. In alternative embodiments, individual cannabinoids such as cannabidiol (CBD) or cannabidiolic acid (CBD-a) are added for therapeutic synergy working together, or in synergy, with the terpenes.

In alternative embodiments, these new synthetic formulations are added to cannabinoid concentrates to re-create products with specific strain characteristics, for example, for the recreational and/or medical cannabis markets. In alternative embodiments, these new synthetic or non-natural terpene formulations are used topically in balms and salves. In alternative embodiments, these new synthetic formulations are used in aerosols, in diffusers or as an aromatherapy. In alternative embodiments, diluting agents such as squalane or 1,3-propanediol are added to the formulations for use in these products of manufacture, for example, in the aerosols, vaporizers, diffusers and the like.

In alternative embodiments, synthetic or non-natural terpene formulations, including pharmaceutical and/or nutraceutical formulations, are administered or delivered using a drug delivery device as provided herein, for example, an inhaler, asthma “puffer-like” device, nebulizer, or the nasal spray device is, or comprises parts of, or is made or used, by a method or a device as described in e.g., U.S. Pat. No. 10,583,261, or 10,561,809 (describing a breath actuated dry powder inhaler with a single air circulation chamber for de-agglomeration of entrained powdered medicament), or U.S. Pat. No. 10,561,807 (describing inhaler devices configured for consuming a defined capacity and generate an aerosol or aerosol imparted with flavor, a sensor configured to detect a predefined variable, an interface configured to make a notification to an inhaler of the aerosol, and a controller), or U.S. Pat. No. 10,463,815 (describing a dry powder inhaler may include a powder storage region, an inlet channel, a dispersion chamber, and an outlet channel); or U.S. patent application publication no. 20200069897 (describing inhalers having a breath actuated trigger mechanism reactive to an inhalation flow to trigger the release of a substance to be inhaled); or 20200061314 (describing a smart inhaler device having a flow pathway comprising a cartridge receptacle that is able to house a cartridge, flow meter, pump, and vaporizer; a wireless communication module; and at least one sensor that captures identifying information related to the cartridge); or 2020004691 (describing dry powder inhalers having replaceable cartridges containing a dry powder for local or systemic delivery through the pulmonary tract and lungs); or 20200046916 (describing an inhaler having a refill assembly comprising: a patient port; a canister actuable by the reusable assembly to deliver a dose of medicament to the patient port, a sleeve which is selectively actuable by a user independently of the reusable assembly so as to act on the canister to deliver a dose of medicament); or 20200046029 (describing an apparatus for generating an aerosol and/or a vapour in an inhaler device includes a reservoir for storing a supply of a liquid; a heating system fluidly connected with the reservoir for receiving the liquid and configured to heat the liquid to generate the aerosol and/or vapor therefrom; a pumping system configured to pump the liquid from the reservoir to the heating system; and a valve arrangement for regulating flow from the pumping system to the heating system); or 20200016345 (describing a dry powder inhaler having a first chamber having an orifice for holding a dry powder and a gas, and a second chamber directly connected to the first chamber by at least one passageway for receiving an aerosolized form of the dry powder from in the first chamber and delivering the aerosolized dry powder to a user). An inhaler as provided herein, or as used in methods as provided herein, can comprise use of a dose counter, e.g., as described in U.S. Pat. No. 10,561,808.

In alternative embodiments, pharmaceutical and nutraceutical formulations or compositions comprise synthetic or non-natural terpene formulations as provided herein for use in in vivo, in vitro or ex vivo methods to treat, prevent, reverse and/or ameliorate conditions or diseases as described herein.

In alternative embodiments, synthetic or non-natural terpene formulations, including pharmaceutical and/or nutraceutical formulations, are administered or delivered by inhalation and/or by an oral, IM or IV formulation, wherein optionally the oral formulation is a liquid, gel, a geltab, a capsule or a tablet) or administration is by subcutaneous administration, sublingual administration, inhalation (optionally by inhalation of a liquid, a tincture, an aerosol, a spray, a mist or a powder), by absorbable patch, by use of an implant, or by use of an enema or a suppository.

In alternative embodiments, the pharmaceutical and nutraceutical formulations or compositions used to practice methods as provided herein can be administered parenterally, topically, orally or by local administration, such as by aerosol or transdermally. These pharmaceutical and nutraceutical formulations or compositions as provided herein can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co., Easton Pa. (“Remington's”). For example, in alternative embodiments, pharmaceutical and nutraceutical formulations or compositions as provided herein are formulated in a buffer, in a saline solution, in a powder, a tincture, an emulsion, in a vesicle, in a liposome, in a nanoparticle, in a nanolipoparticle, a pill, a tablet, a gel, a geltab, a capsule, a liquid, and the like. In alternative embodiments, the pharmaceutical and nutraceutical formulations or compositions as provided herein are formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods as provided herein can be in forms such as tablets, pills, powders, tinctures, capsules, liquids, gels, geltabs, syrups, slurries, suspensions, etc., suitable for in vivo, in vitro or ex vivo applications.

In alternative embodiment, pharmaceutical and nutraceutical formulations or compositions as provided herein used to practice methods as provided herein can comprise a solution of compositions disposed in or dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

Pharmaceutical and nutraceutical formulations or compositions as provided herein used to practice methods as provided herein can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The pharmaceutical and nutraceutical formulations or compositions as provided herein used to practice methods as provided herein can be delivered by the use of liposomes. By using liposomes, particularly where the liposome surface carries ligands specific for target cells (e.g., an injured or diseased neuronal cell or CNS tissue), or are otherwise preferentially directed to a specific tissue or organ type, one can focus the delivery of the active agent into a target cells in an in vivo, in vitro or ex vivo application.

Nanoparticles, Nanolipoparticles and Liposomes

Also provided are nanoparticles, nanolipoparticles, vesicles and liposomal membranes comprising pharmaceutical and nutraceutical formulations or compositions as provided herein used to practice methods as provided herein.

Provided are multilayered liposomes comprising pharmaceutical and nutraceutical formulations or compositions as provided herein used to practice methods as provided herein, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, e.g., as described in Park, et al., U.S. Pat. Pub. No. 20070042031, including method of producing a liposome by encapsulating an active agent (e.g., synthetic or non-natural formulations comprising a non-natural mix of bio-mass or plant-derived terpenes or terpenoids as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, e.g., for targeting delivery of a compound (e.g., pharmaceutical and nutraceutical formulations or compositions as provided herein), as described e.g., in U.S. Pat. Pub. No. 20070110798.

The invention also provides nanoparticles comprising compounds (e.g., pharmaceutical and nutraceutical formulations or compositions as provided herein) used to practice methods as provided herein in the form of active agent-containing nanoparticles (e.g., a secondary nanoparticle), as described, e.g., in U.S. Pat. Pub. No. 20070077286. In one embodiment, provided are nanoparticles comprising a fat-soluble active agent of this invention or a fat-solubilized water-soluble active agent to act with a bivalent or trivalent metal salt.

In one embodiment, solid lipid suspensions can be used to formulate and to deliver compositions used to practice methods as provided herein to mammalian cells in vivo, in vitro or ex vivo, as described, e.g., in U.S. Pat. Pub. No. 20050136121.

Dosaging

The pharmaceutical compositions and formulations, including nutraceutical formulations, used to practice methods as provided herein can be administered for prophylactic and/or therapeutic treatments. In alternative embodiments, therapeutic applications, compositions are administered to a subject already suffering from a disease or condition as described herein (a “therapeutically effective amount”). For example, in alternative embodiments, polypeptides used to practice methods as provided herein are administered to an individual in need thereof in an amount sufficient to treat, prevent, reverse and/or ameliorate a disease or condition as described herein.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice the invention are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, as a tincture or other topical, a spray or by inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, e.g., compositions for treating any neurological or neuromuscular disease, condition, infection or injury, including related inflammatory and autoimmune diseases and conditions, and the like. For example, compositions used to practice methods as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (e.g., a ficolin), carbohydrate-binding domains, and the like and combinations thereof.

In alternative embodiments, pharmaceutical compositions and formulations, including nutraceutical formulations, for sublingual administration with oral spray or gels, geltabs, capsules or softgel caps or equivalent are formulated as 1% to 2.5% of total infusion of terpenes, and optionally at a dosage of 5 mg/g to 50 mg/g dosage per 1 mL once a day.

In alternative embodiments, pharmaceutical compositions and formulations, including nutraceutical formulations, for inhalation are formulated as a concentrate of 1% to 10% of total infusion of terpenes, and optionally administered at a dosage of 5 mg/g to 50 mg/g of dosage per 1 mL once a day.

In alternative embodiments, pharmaceutical compositions and formulations, including nutraceutical formulations, are formulated as a tincture with pure plant based oils, optionally formulated at a 1% to 2.5% of total infusion of terpenes and optionally at a dosage of 30 mg/g to 2500 mg/g of dosage per 1 mL once a day.

Exemplary Formulations

### Formula #1

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; Guaiol; D-Limonene; Geranyl Acetate; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 0.52 (mg/g), Camphene 0.057 (mg/g), alpha-Pinene 0.262 (mg/g), alpha-Phellandrene 0.011 (mg/g), gamma-Terpinene 0.002 (mg/g), alpha-Terpinene 0.004 (mg/g), p-Cymene 0.014 (mg/g), Terpinolene 0.234 (mg/g), Fenchone 0.006 (mg/g), beta-Pinene 0.413 (mg/g), Fenchol 0.139 (mg/g), Terpineol 0.06 (mg/g), 3-Carene 0.015 (mg/g), Myrcene 3.74 (mg/g), Borneol 0.005 (mg/g), Guaiol 0.003 (mg/g), D-Limonene 3.309 (mg/g), Geranyl Acetate 0.002 (mg/g), Caryophyllene oxide 0.049 (mg/g), BetaCaryophyllene 0.605 (mg/g), alpha-Farnesene 0.029 (mg/g), beta-Farnesene 0.04 (mg/g), Humulene 0.142 (mg/g), beta-Ocimene 0.059 (mg/g), alpha-Ocimene 0.006 (mg/g), Sabinene Hydrate 0.004 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Neuroprotective, Immunosuppressive, Appetite Stimulant, Bronchodilator, Anti-ischemic, Anti-psychotic, Anti-emetic, Anti-anxiety (Anxiolytic).

### Formula #2

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; Terpinolene; Fenchone; beta-Pinene; Fenchol; Terpineol; 3-Carene; Myrcene; Borneol; Cedrol; Guaiol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Eucalyptol 0.019 (mg/g), Linalool 0.108 (mg/g), Camphene 0.02 (mg/g), alpha-Pinene 0.612 (mg/g), alpha-Phellandrene 0.006 (mg/g), gamma-Terpinene 0.002 (mg/g), alpha-Terpinene 0.002 (mg/g), Terpinolene 0.101 (mg/g), Fenchone 0.007 (mg/g), beta-Pinene 0.347 (mg/g), Fenchol 0.084 (mg/g), Terpineol 0.021 (mg/g), 3-Carene 0.005 (mg/g), Myrcene 1.757 (mg/g), Borneol 0.005 (mg/g), Cedrol 0.004 (mg/g), Guaiol 0.005 (mg/g), D-Limonene 0.715 (mg/g), Caryophyllene oxide 0.064 (mg/g), BetaCaryophyllene 3.641 (mg/g), alpha-Farnesene 0.196 (mg/g), beta-Farnesene 0.439 (mg/g), Humulene 1.107 (mg/g), beta-Ocimene 0.084 (mg/g), alpha-Ocimene 0.007 (mg/g), Sabinene Hydrate 0.003 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-psychotic, Bronchodilator, Neuroprotective, Anti-emetic, Immunosuppressive, Anti-anxiety (Anxiolytic), Anti-ischemic, Appetite Stimulant.

### Formula #3

(a) one, two, three, 4, 5, 6, 7, 8, 9, 10 or all of the following terepenes: Menthol;

Linalool; Camphene; alpha-Pinene; gamma-Terpinene; Terpinolene; Fenchone; beta-Pinene; Fenchol; Terpineol; Myrcene; Borneol; D-Limonene; Caryophyllene oxide; BetaCaryophyllene; alpha-Farnesene; Humulene; Sabinene Hydrate;

Optionally formulated as: Menthol 0.001 (mg/g), Linalool 0.28 (mg/g), Camphene 0.02 (mg/g), alpha-Pinene 0.751 (mg/g), gamma-Terpinene 0.001 (mg/g), Terpinolene 0.014 (mg/g), Fenchone 0.005 (mg/g), beta-Pinene 0.389 (mg/g), Fenchol 0.046 (mg/g), Terpineol 0.015 (mg/g), Myrcene 7.461 (mg/g), Borneol 0.003 (mg/g), D-Limonene 0.618 (mg/g), Caryophyllene oxide 0.003 (mg/g), BetaCaryophyllene 0.28 (mg/g), alpha-Farnesene 0.01 (mg/g), Humulene 0.066 (mg/g), Sabinene Hydrate 0.003 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-anxiety (Anxiolytic), Anti-emetic, Appetite Stimulant, Neuroprotective, Immunosuppressive, Anti-psychotic, Anti-spasmotic, Bronchodilator.

### Formula #4

(a) one, two, three, 4, 5, 6, 7, 8, 9, 10 or all of the following terepenes: Eucalyptol; Linalool; Camphene; alpha-Pinene; p-Cymene; Terpinolene; Fenchone; beta-Pinene; Fenchol; Terpineol; Myrcene; Borneol; Cedrol; D-Limonene; Caryophyllene oxide; BetaCaryophyllene; Humulene; beta-Ocimene;

Optionally formulated as: Eucalyptol 0.027 (mg/g), Linalool 0.087 (mg/g), Camphene 0.048 (mg/g), alpha-Pinene 0.946 (mg/g), p-Cymene 0.006 (mg/g), Terpinolene 0.018 (mg/g), Fenchone 0.013 (mg/g), beta-Pinene 0.809 (mg/g), Fenchol 0.047 (mg/g), Terpineol 0.006 (mg/g), Myrcene 5.835 (mg/g), Borneol 0.002 (mg/g), Cedrol 0.001 (mg/g), D-Limonene 1.581 (mg/g), Caryophyllene oxide 0.029 (mg/g), BetaCaryophyllene 0.356 (mg/g), Humulene 0.059 (mg/g), beta-Ocimene 0.124 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-spasmotic, Anti-emetic, Bronchodilator, Anti-ischemic, Immunosuppressive, Anti-psychotic, Appetite Stimulant, Anti-anxiety (Anxiolytic).

### Formula #5

(a) one, two, three, 4, 5, 6, 7, 8, 9, 10 or all of the following terepenes: Linalool; Camphene; alpha-Pinene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene; alpha-Ocimene;

Optionally formulated as: Linalool 0.908 (mg/g), Camphene 0.062 (mg/g), alpha-Pinene 0.29 (mg/g), Terpinolene 0.036 (mg/g), Fenchone 0.03 (mg/g), beta-Pinene 0.418 (mg/g), Fenchol 0.183 (mg/g), Terpineol 0.09 (mg/g), Myrcene 2.456 (mg/g), Borneol 0.008 (mg/g), D-Limonene 3.322 (mg/g), Caryophyllene oxide 0.03 (mg/g), BetaCaryophyllene 1.433 (mg/g), alpha-Farnesene 0.059 (mg/g), beta-Farnesene 0.055 (mg/g), Humulene 0.371 (mg/g), beta-Ocimene 0.168 (mg/g), alpha-Ocimene 0.011 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-anxiety (Anxiolytic), Anti-spasmotic, Anti-ischemic, Anti-emetic, Bronchodilator, Anti-psychotic, Neuroprotective, Immunosuppressive.

### Formula #6

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene;

Optionally formulated as: Linalool 0.33 (mg/g), Camphene 0.028 (mg/g), alpha-Pinene 0.711 (mg/g), alpha-Phellandrene 0.016 (mg/g), gamma-Terpinene 0.004 (mg/g), alpha-Terpinene 0.007 (mg/g), p-Cymene 0.006 (mg/g), Terpinolene 0.375 (mg/g), Fenchone 0.019 (mg/g), beta-Pinene 0.41 (mg/g), Fenchol 0.073 (mg/g), Terpineol 0.04 (mg/g), 3-Carene 0.014 (mg/g), Myrcene 5.758 (mg/g), Borneol 0.004 (mg/g), Cedrol 0.001 (mg/g), D-Limonene 1.477 (mg/g), Caryophyllene oxide 0.016 (mg/g), BetaCaryophyllene 0.459 (mg/g), alpha-Farnesene 0.032 (mg/g), beta-Farnesene 0.039 (mg/g), Humulene 0.11 (mg/g), beta-Ocimene 0.043 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Bronchodilator, Anti-psychotic, Anti-anxiety (Anxiolytic), Appetite Stimulant, Neuroprotective, Anti-spasmotic, Anti-emetic, Anti-ischemic.

### Formula #7

(a) one, two, three, 4, 5, 6, 7, 8, 9, 10 or all of the following terepenes: Camphor; Eucalyptol; Linalool; Camphene; alpha-Pinene; gamma-Terpinene; Terpinolene; Fenchone; beta-Pinene; Fenchol; Terpineol; Myrcene; Borneol; Alloaromadendrene; D-Limonene; Caryophyllene oxide; BetaCaryophyllene; alpha-Farnesene; beta-Farnesene; Humulene; Sabinene Hydrate;

Optionally formulated as: Camphor 0.001 (mg/g), Eucalyptol 0.012 (mg/g), Linalool 0.259 (mg/g), Camphene 0.03 (mg/g), alpha-Pinene 1.794 (mg/g), gamma-Terpinene 0.002 (mg/g), Terpinolene 0.012 (mg/g), Fenchone 0.007 (mg/g), beta-Pinene 0.792 (mg/g), Fenchol 0.048 (mg/g), Terpineol 0.019 (mg/g), Myrcene 5.55 (mg/g), Borneol 0.003 (mg/g), Alloaromadendrene 0.019 (mg/g), D-Limonene 0.522 (mg/g), Caryophyllene oxide 0.002 (mg/g), BetaCaryophyllene 0.601 (mg/g), alpha-Farnesene 0.012 (mg/g), beta-Farnesene 0.05 (mg/g), Humulene 0.142 (mg/g), Sabinene Hydrate 0.004 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Bronchodilator, Neuroprotective, Anti-anxiety (Anxiolytic), Anti-ischemic, Anti-emetic, Appetite Stimulant, Anti-psychotic, Anti-spasmotic.

### Formula #8

(a) one, two, three, 4, 5, 6, 7, 8, 9, 10 or all of the following terepenes: Linalool; Camphene; alpha-Pinene; Terpinolene; Fenchone; beta-Pinene; Fenchol; Terpineol; Myrcene; Borneol; D-Limonene; BetaCaryophyllene; Humulene;

Optionally formulated as: Linalool 0.237 (mg/g), Camphene 0.02 (mg/g), alpha-Pinene 0.685 (mg/g), Terpinolene 0.012 (mg/g), Fenchone 0.004 (mg/g), beta-Pinene 0.331 (mg/g), Fenchol 0.035 (mg/g), Terpineol 0.016 (mg/g), Myrcene 7.778 (mg/g), Borneol 0.002 (mg/g), D-Limonene 0.621 (mg/g), BetaCaryophyllene 0.209 (mg/g), Humulene 0.047 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Neuroprotective, Anti-spasmotic, Anti-psychotic, Anti-anxiety (Anxiolytic), Immunosuppressive, Anti-emetic, Appetite Stimulant, Anti-ischemic.

### Formula #9

(a) one, two, three, 4, 5, 6, 7, 8, 9, 10 or all of the following terepenes: Eucalyptol; Linalool; Camphene; alpha-Pinene; Terpinolene; Fenchone; beta-Pinene; Fenchol; Terpineol; Myrcene; Borneol; D-Limonene; Caryophyllene oxide; BetaCaryophyllene; alpha-Farnesene; Humulene; beta-Ocimene; Sabinene Hydrate;

Optionally formulated as: Eucalyptol 0.014 (mg/g), Linalool 0.111 (mg/g), Camphene 0.031 (mg/g), alpha-Pinene 1.81 (mg/g), Terpinolene 0.013 (mg/g), Fenchone 0.01 (mg/g), beta-Pinene 0.794 (mg/g), Fenchol 0.069 (mg/g), Terpineol 0.018 (mg/g), Myrcene 5.471 (mg/g), Borneol 0.005 (mg/g), D-Limonene 0.875 (mg/g), Caryophyllene oxide 0.001 (mg/g), BetaCaryophyllene 0.451 (mg/g), alpha-Farnesene 0.023 (mg/g), Humulene 0.093 (mg/g), beta-Ocimene 0.124 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-psychotic, Anti-spasmotic, Anti-emetic, Bronchodilator, Anti-anxiety (Anxiolytic), Appetite Stimulant, Immunosuppressive, Neuroprotective.

### Formula #10

(a) one, two, three, 4, 5, 6, 7, 8, 9, 10 or all of the following terepenes: Eucalyptol; Linalool; Camphene; alpha-Pinene; p-Cymene; Terpinolene; beta-Pinene; Fenchol; Myrcene; Borneol; D-Limonene; Caryophyllene oxide; BetaCaryophyllene; Humulene; beta-Ocimene;

Optionally formulated as: Eucalyptol 0.04 (mg/g), Linalool 0.168 (mg/g), Camphene 0.022 (mg/g), alpha-Pinene 1.059 (mg/g), p-Cymene 0.006 (mg/g), Terpinolene 0.013 (mg/g), beta-Pinene 0.353 (mg/g), Fenchol 0.007 (mg/g), Myrcene 7.305 (mg/g), Borneol 0.001 (mg/g), D-Limonene 0.384 (mg/g), Caryophyllene oxide 0.02 (mg/g), BetaCaryophyllene 0.152 (mg/g), Humulene 0.024 (mg/g), beta-Ocimene 0.441 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Immunosuppressive, Anti-spasmotic, Anti-anxiety (Anxiolytic), Anti-psychotic, Neuroprotective, Anti-ischemic, Appetite Stimulant, Anti-emetic.

### Formula #11

(a) one, two, three, 4, 5, 6, 7, 8, 9, 10 or all of the following terepenes: alpha-Pinene; gamma-Terpinene; alpha-Terpinene; 4-Carvomenthenol; Terpinolene; beta-Pinene; Terpineol; Valencene; Sabinene Hydrate; Camphor; Linalool; Camphene; alpha-Phellandrene; p-Cymene; Fenchone; Fenchol; 3-Carene; Myrcene; Borneol; D-Menthol; D-Limonene; Caryophyllene oxide; Bisabolene; BetaCaryophyllene; alpha-Farnesene; beta-Farnesene; Humulene; beta-Ocimene; Nerolidol; cis-Nerolidol; alpha-Ocimene;

Optionally formulated as: alpha-Pinene 26.01 (mg/g), gamma-Terpinene 0.45 (mg/g), alpha-Terpinene 0.51 (mg/g), 4-Carvomenthenol 0.45 (mg/g), Terpinolene 31.2 (mg/g), beta-Pinene 29.57 (mg/g), Terpineol 7.95 (mg/g), Valencene 2.45 (mg/g), Sabinene Hydrate 0.41 (mg/g), Camphor 0.65 (mg/g), Linalool 73.42 (mg/g), Camphene 5.78 (mg/g), alpha-Phellandrene 1.27 (mg/g), p-Cymene 0.26 (mg/g), Fenchone 2.43 (mg/g), Fenchol 25.68 (mg/g), 3-Carene 1.26 (mg/g), Myrcene 144.06 (mg/g), Borneol 3.99 (mg/g), D-Menthol 0.49 (mg/g), D-Limonene 297.35 (mg/g), Caryophyllene oxide 0.3 (mg/g), Bisabolene 23.47 (mg/g), BetaCaryophyllene 76.55 (mg/g), alpha-Farnesene 2.47 (mg/g), beta-Farnesene 2 (mg/g), Humulene 18.16 (mg/g), beta-Ocimene 2.95 (mg/g), Nerolidol 0.52 (mg/g), cis-Nerolidol 1.43 (mg/g), alpha-Ocimene 0.68 (mg/g),

(b) in alternative embodiment, this formulation is used to treat, ameliorate, lesson the symptoms of, or prevent any one of the following conditions, or be used as an: Anti-inflammatory, Anti-bacterial, Anti-insomnia, Anti-depressant, Anti-convulsive, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Bronchodilator, Anti-ischemic, Appetite Stimulant, Anti-emetic, Immunosuppressive, Anti-anxiety (Anxiolytic), Anti-psychotic, Anti-spasmotic.

### Formula #12

(a) one, two, three, 4, 5, 6, 7, 8, 9, 10 or all of the following terepenes: Menthol; Eucalyptol; Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Menthol 0.0005 (mg/g), Eucalyptol 0.026 (mg/g), Linalool 0.0635 (mg/g), Camphene 0.0395 (mg/g), alpha-Pinene 0.3395 (mg/g), gamma-Terpinene 0.0015 (mg/g), p-Cymene 0.008 (mg/g), Terpinolene 0.0515 (mg/g), Fenchone 0.0055 (mg/g), beta-Pinene 0.4675 (mg/g), Fenchol 0.052 (mg/g), Terpineol 0.0045 (mg/g), Myrcene 4.873 (mg/g), Borneol 0.001 (mg/g), Cedrol 0.001 (mg/g), D-Limonene 3.071 (mg/g), Caryophyllene oxide 0.0385 (mg/g), BetaCaryophyllene 0.68 (mg/g), alpha-Farnesene 0.008 (mg/g), beta-Farnesene 0.004 (mg/g), Humulene 0.106 (mg/g), beta-Ocimene 0.0845 (mg/g), alpha-Ocimene 0.0095 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-spasmotic, Anti-anxiety (Anxiolytic), Neuroprotective, Anti-psychotic, Immunosuppressive, Anti-ischemic, Anti-emetic, Bronchodilator.

### Formula #13

(a) one, two, three, 4, 5, 6, 7, 8, 9, 10 or all of the following terepenes: Camphor; Linalool; Camphene; alpha-Pinene; Citronellol; Terpinolene; Fenchone; beta-Pinene; Fenchol; Terpineol; Myrcene; Borneol; D-Limonene; Caryophyllene oxide; Bisabolene; BetaCaryophyllene; alpha-Farnesene; beta-Farnesene; Humulene; beta-Ocimene; cis-Nerolidol; Valencene;

Optionally formulated as: Camphor 0.3 (mg/g), Linalool 23.9 (mg/g), Camphene 2.5 (mg/g), alpha-Pinene 57.9 (mg/g), Citronellol 0.9 (mg/g), Terpinolene 2.5 (mg/g), Fenchone 1.7 (mg/g), beta-Pinene 35.6 (mg/g), Fenchol 9.4 (mg/g), Terpineol 4.4 (mg/g), Myrcene 288.8 (mg/g), Borneol 1.8 (mg/g), D-Limonene 85.4 (mg/g), Caryophyllene oxide 0.9 (mg/g), Bisabolene 17.2 (mg/g), BetaCaryophyllene 110.2 (mg/g), alpha-Farnesene 7.5 (mg/g), beta-Farnesene 14.7 (mg/g), Humulene 25.8 (mg/g), beta-Ocimene 6.9 (mg/g), cis-Nerolidol 2.4 (mg/g), Valencene 4.4 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Anti-spasmotic, Anti-emetic, Neuroprotective, Bronchodilator, Anti-anxiety (Anxiolytic), Anti-psychotic, Anti-ischemic, Appetite Stimulant, Immunosuppressive.

### Formula #14

(a) one, two, three, 4, 5, 6, 7, 8, 9, 10 or all of the following terepenes: Eucalyptol; Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; Humulene; beta-Ocimene; alpha-Ocimene;

Optionally formulated as: Eucalyptol 0.028 (mg/g), Linalool 0.064 (mg/g), Camphene 0.039 (mg/g), alpha-Pinene 0.339 (mg/g), gamma-Terpinene 0.001 (mg/g), p-Cymene 0.008 (mg/g), Terpinolene 0.052 (mg/g), Fenchone 0.006 (mg/g), beta-Pinene 0.467 (mg/g), Fenchol 0.052 (mg/g), Terpineol 0.004 (mg/g), Myrcene 4.88 (mg/g), Borneol 0.001 (mg/g), Cedrol 0.001 (mg/g), D-Limonene 3.072 (mg/g), Caryophyllene oxide 0.038 (mg/g), BetaCaryophyllene 0.68 (mg/g), alpha-Farnesene 0.008 (mg/g), Humulene 0.106 (mg/g), beta-Ocimene 0.084 (mg/g), alpha-Ocimene 0.01 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Neuroprotective, Anti-spasmotic, Anti-psychotic, Bronchodilator, Anti-anxiety (Anxiolytic), Anti-emetic, Appetite Stimulant, Immunosuppressive.

### Formula #15

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; Humulene; beta-Ocimene; alpha-Ocimene;

Optionally formulated as: Eucalyptol 0.023 (mg/g), Linalool 0.107 (mg/g), Camphene 0.048 (mg/g), alpha-Pinene 1.095 (mg/g), alpha-Phellandrene 0.001 (mg/g), gamma-Terpinene 0.002 (mg/g), p-Cymene 0.009 (mg/g), Terpinolene 0.022 (mg/g), Fenchone 0.007 (mg/g), beta-Pinene 1.139 (mg/g), Fenchol 0.075 (mg/g), Terpineol 0.016 (mg/g), 3-Carene 0.003 (mg/g), Myrcene 4.365 (mg/g), Borneol 0.002 (mg/g), Cedrol 0.002 (mg/g), D-Limonene 2.314 (mg/g), Caryophyllene oxide 0.06 (mg/g), BetaCaryophyllene 0.546 (mg/g), alpha-Farnesene 0.012 (mg/g), Humulene 0.096 (mg/g), beta-Ocimene 0.041 (mg/g), alpha-Ocimene 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Neuroprotective, Anti-emetic, Immunosuppressive, Bronchodilator, Anti-psychotic, Anti-ischemic, Appetite Stimulant, Anti-spasmotic.

### Formula #16

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; Isopulegol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Eucalyptol 0.022 (mg/g), Linalool 0.327 (mg/g), Camphene 0.049 (mg/g), alpha-Pinene 2.059 (mg/g), alpha-Phellandrene 0.004 (mg/g), gamma-Terpinene 0.003 (mg/g), p-Cymene 0.003 (mg/g), Terpinolene 0.065 (mg/g), Fenchone 0.009 (mg/g), beta-Pinene 0.94 (mg/g), Fenchol 0.148 (mg/g), Terpineol 0.048 (mg/g), 3-Carene 0.003 (mg/g), Myrcene 1.265 (mg/g), Borneol 0.009 (mg/g), Cedrol 0.001 (mg/g), Isopulegol 0.008 (mg/g), D-Limonene 1.089 (mg/g), Caryophyllene oxide 0.029 (mg/g), BetaCaryophyllene 2.67 (mg/g), alpha-Farnesene 0.099 (mg/g), beta-Farnesene 0.231 (mg/g), Humulene 0.768 (mg/g), beta-Ocimene 0.019 (mg/g), alpha-Ocimene 0.006 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-spasmotic, Anti-psychotic, Immunosuppressive, Appetite Stimulant, Anti-emetic, Anti-ischemic, Anti-anxiety (Anxiolytic), Neuroprotective

### Formula #17

(a) one, two, three, 4, 5, 6, 7, 8, 9, 10 or all of the following terepenes: Cedrol; Guaiol; Caryophyllene oxide; BetaCaryophyllene; alpha-Farnesene; beta-Farnesene; Humulene; cis-Nerolidol; Eucalyptol; Linalool; Camphene; alpha-Pinene; alpha-Phellandrene; gamma-Terpinene; alpha-Terpinene; p-Cymene; Terpinolene; gamma-Terpineol; Fenchone; beta-Pinene; Fenchol; Limonene; 3-Carene; Myrcene; Borneol; beta-Ocimene; trans-beta-Ocimene;

Optionally formulated as: Cedrol 0.1 (mg/g), Guaiol 0.1 (mg/g), Caryophyllene oxide 1.7 (mg/g), BetaCaryophyllene 130.1 (mg/g), alpha-Farnesene 11.2 (mg/g), beta-Farnesene 38.5 (mg/g), Humulene 27.2 (mg/g), cis-Nerolidol 0.5 (mg/g), Eucalyptol 1.9 (mg/g), Linalool 2.2 (mg/g), Camphene 1.9 (mg/g), alpha-Pinene 27.5 (mg/g), alpha-Phellandrene 0.9 (mg/g), gamma-Terpinene 0.7 (mg/g), alpha-Terpinene 0.6 (mg/g), p-Cymene 0.4 (mg/g), Terpinolene 27.8 (mg/g), gamma-Terpineol 1.2 (mg/g), Fenchone 0.8 (mg/g), beta-Pinene 40.6 (mg/g), Fenchol 7.2 (mg/g), Limonene 252.7 (mg/g), 3-Carene 0.6 (mg/g), Myrcene 323 (mg/g), Borneol 0.5 (mg/g), beta-Ocimene 32.2 (mg/g), trans-beta-Ocimene 2.1 (mg/g),

(b) in alternative embodiment, this formulation is used to treat, ameliorate, lesson the symptoms of, or prevent any one of the following conditions, or be used as an: Anti-inflammatory, Anti-bacterial, Anti-cancer, Appetite Suppressant, Anti-depressant, Anti-insomnia, Anti-convulsive, Analgesic,

(c) in alternative embodiment, this formulation is used to treat, ameliorate, lesson the symptoms of, or prevent any one of the following conditions, or be used as an: Anti-ischemic, Anti-psychotic, Anti-spasmotic, Immunosuppressive, Anti-emetic, Anti-anxiety (Anxiolytic), Bronchodilator, Neuroprotective, Appetite Stimulant

### Formula #18

(a) one, two, three, 4, 5, 6, 7, 8, 9, 10 or all of the following terepenes: Linalool; Camphene; alpha-Pinene; alpha-Phellandrene; Terpinolene; Fenchone; beta-Pinene; Fenchol; Terpineol; 3-Carene; Myrcene; Borneol; D-Limonene; Caryophyllene oxide; BetaCaryophyllene; alpha-Farnesene; Humulene; beta-Ocimene; Sabinene Hydrate; Optionally formulated as: Linalool 0.15 (mg/g), Camphene 0.017 (mg/g), alpha-Pinene 0.548 (mg/g), alpha-Phellandrene 0.003 (mg/g), Terpinolene 0.076 (mg/g), Fenchone 0.005 (mg/g), beta-Pinene 0.227 (mg/g), Fenchol 0.034 (mg/g), Terpineol 0.01 (mg/g), 3-Carene 0.002 (mg/g), Myrcene 7.197 (mg/g), Borneol 0.003 (mg/g), D-Limonene 0.48 (mg/g), Caryophyllene oxide 0.005 (mg/g), BetaCaryophyllene 0.626 (mg/g), alpha-Farnesene 0.022 (mg/g), Humulene 0.131 (mg/g), beta-Ocimene 0.405 (mg/g), Sabinene Hydrate 0.003 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-psychotic, Anti-emetic, Bronchodilator, Appetite Stimulant, Anti-spasmotic, Anti-anxiety (Anxiolytic), Neuroprotective, Immunosuppressive

### Formula #19

(a) one, two, three, 4, 5, 6, 7, 8, 9, 10 or all of the following terepenes: Camphor; Eucalyptol; Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Camphor 0.001 (mg/g), Eucalyptol 0.001 (mg/g), Linalool 0.1 (mg/g), Camphene 0.056 (mg/g), alpha-Pinene 0.232 (mg/g), gamma-Terpinene 0.001 (mg/g), p-Cymene 0.004 (mg/g), Terpinolene 0.03 (mg/g), Fenchone 0.011 (mg/g), beta-Pinene 0.362 (mg/g), Fenchol 0.145 (mg/g), Terpineol 0.053 (mg/g), Myrcene 3.491 (mg/g), Borneol 0.006 (mg/g), Cedrol 0.002 (mg/g), D-Limonene 4.495 (mg/g), Caryophyllene oxide 0.044 (mg/g), BetaCaryophyllene 0.686 (mg/g), alpha-Farnesene 0.031 (mg/g), beta-Farnesene 0.02 (mg/g), Humulene 0.154 (mg/g), beta-Ocimene 0.014 (mg/g), alpha-Ocimene 0.01 (mg/g), Sabinene Hydrate 0.003 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Bronchodilator, Appetite Stimulant, Anti-psychotic, Anti-anxiety (Anxiolytic), Anti-emetic, Neuroprotective, Immunosuppressive, Anti-ischemic

### Formula #20

(a) one, two, three, 4, 5, 6, 7, 8, 9, 10 or all of the following terepenes: Linalool; Fenchol; Terpineol; alpha-Cedrene; alpha-Bisabolol; Caryophyllene oxide; BetaCaryophyllene; Humulene; cis-Nerolidol; Valencene; Eucalyptol; Camphene; alpha-Pinene; gamma-Terpinene; alpha-Terpinene; p-Cymene; Terpinolene; beta-Pinene; 3-Carene; Myrcene; D-Limonene;

Optionally formulated as: Linalool 0.87 (mg/g), Fenchol 0.71 (mg/g), Terpineol 0.21 (mg/g), alpha-Cedrene 0.93 (mg/g), alpha-Bisabolol 0.04 (mg/g), Caryophyllene oxide 0.47 (mg/g), BetaCaryophyllene 26.28 (mg/g), Humulene 5.14 (mg/g), cis-Nerolidol 0.33 (mg/g), Valencene 0.97 (mg/g), Eucalyptol 0.34 (mg/g), Camphene 1.35 (mg/g), alpha-Pinene 56.81 (mg/g), gamma-Terpinene 0.23 (mg/g), alpha-Terpinene 0.28 (mg/g), p-Cymene 0.06 (mg/g), Terpinolene 6.39 (mg/g), beta-Pinene 56.55 (mg/g), 3-Carene 0.27 (mg/g), Myrcene 796.48 (mg/g), D-Limonene 39.65 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Appetite Suppressant, Analgesic,

(c) in alternative embodiment, this formulation is used to treat, ameliorate, lesson the symptoms of, or prevent any one of the following conditions, or be used as an: Anti-psychotic, Anti-emetic, Anti-anxiety (Anxiolytic), Appetite Stimulant, Anti-ischemic, Neuroprotective, Immunosuppressive, Bronchodilator, Anti-spasmotic

### Formula #21

(a) one, two, three, 4, 5, 6, 7, 8, 9, 10 or all of the following terepenes: Linalool; Camphene; alpha-Pinene; gamma-Terpinene; p-Cymene; Terpinolene; Fenchone; beta-Pinene; Fenchol; Terpineol; Myrcene; Borneol; D-Limonene; Caryophyllene oxide; BetaCaryophyllene; Humulene; beta-Ocimene; alpha-Ocimene;

Optionally formulated as: Linalool 0.158 (mg/g), Camphene 0.071 (mg/g), alpha-Pinene 0.187 (mg/g), gamma-Terpinene 0.001 (mg/g), p-Cymene 0.004 (mg/g), Terpinolene 0.034 (mg/g), Fenchone 0.018 (mg/g), beta-Pinene 0.566 (mg/g), Fenchol 0.133 (mg/g), Terpineol 0.017 (mg/g), Myrcene 2.388 (mg/g), Borneol 0.003 (mg/g), D-Limonene 6.095 (mg/g), Caryophyllene oxide 0.014 (mg/g), BetaCaryophyllene 0.237 (mg/g), Humulene 0.04 (mg/g), beta-Ocimene 0.005 (mg/g), alpha-Ocimene 0.011 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Immunosuppressive, Appetite Stimulant, Neuroprotective, Anti-spasmotic, Anti-ischemic, Bronchodilator, Anti-psychotic, Anti-emetic

### Formula #22

(a) one, two, three, 4, 5, 6, 7, 8, 9, 10 or all of the following terepenes: Camphor; Linalool; Camphene; alpha-Pinene; alpha-Phellandrene; alpha-Terpinene; p-Cymene; Terpinolene; Fenchone; beta-Pinene; Fenchol; Terpineol; 3-Carene; Myrcene; Borneol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Camphor 0.001 (mg/g), Linalool 0.193 (mg/g), Camphene 0.029 (mg/g), alpha-Pinene 0.746 (mg/g), alpha-Phellandrene 0.016 (mg/g), alpha-Terpinene 0.006 (mg/g), p-Cymene 0.002 (mg/g), Terpinolene 0.341 (mg/g), Fenchone 0.013 (mg/g), beta-Pinene 0.369 (mg/g), Fenchol 0.076 (mg/g), Terpineol 0.055 (mg/g), 3-Carene 0.012 (mg/g), Myrcene 6.223 (mg/g), Borneol 0.005 (mg/g), D-Limonene 1.003 (mg/g), Caryophyllene oxide 0.003 (mg/g), BetaCaryophyllene 0.337 (mg/g), alpha-Farnesene 0.027 (mg/g), beta-Farnesene 0.045 (mg/g), Humulene 0.096 (mg/g), beta-Ocimene 0.368 (mg/g), alpha-Ocimene 0.005 (mg/g), Sabinene Hydrate 0.004 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-emetic, Anti-ischemic, Appetite Stimulant, Immunosuppressive, Anti-anxiety (Anxiolytic), Bronchodilator, Anti-psychotic, Neuroprotective

### Formula #23

(a) one, two, three, 4, 5, 6, 7, 8, 9, 10 or all of the following terepenes: Camphor; Linalool; 4-Carvomenthenol; Fenchol; Terpineol; Borneol; Alloaromadendrene; Caryophyllene oxide; Bisabolene; BetaCaryophyllene; alpha-Farnesene; beta-Farnesene; Humulene; cis-Nerolidol; Valencene; Camphene; alpha-Pinene; Terpinolene; Fenchone; beta-Pinene; Myrcene; D-Limonene; beta-Ocimene; Optionally formulated as: Camphor 0.4 (mg/g), Linalool 10.4 (mg/g), 4-Carvomenthenol 0.3 (mg/g), Fenchol 13.5 (mg/g), Terpineol 8.5 (mg/g), Borneol 3.6 (mg/g), Alloaromadendrene 0.6 (mg/g), Caryophyllene oxide 3.7 (mg/g), Bisabolene 32.9 (mg/g), BetaCaryophyllene 203.9 (mg/g), alpha-Farnesene 9.2 (mg/g), beta-Farnesene 32.5 (mg/g), Humulene 53.3 (mg/g), cis-Nerolidol 3.6 (mg/g), Valencene 11.4 (mg/g), Camphene 2.4 (mg/g), alpha-Pinene 84.5 (mg/g), Terpinolene 2.4 (mg/g), Fenchone 3.3 (mg/g), beta-Pinene 47.7 (mg/g), Myrcene 181.8 (mg/g), D-Limonene 39.3 (mg/g), beta-Ocimene 1.4 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-inflammatory, Anti-cancer, Appetite Suppressant,

(c) in alternative embodiment, this formulation is used to treat, ameliorate, lesson the symptoms of, or prevent any one of the following conditions, or be used as an: Immunosuppressive, Appetite Stimulant, Anti-psychotic, Bronchodilator, Anti-ischemic, Anti-spasmotic, Anti-anxiety (Anxiolytic), Neuroprotective, Anti-emetic

### Formula #24

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene; Optionally formulated as: Eucalyptol 0.023 (mg/g), Linalool 0.1065 (mg/g), Camphene 0.048 (mg/g), alpha-Pinene 1.1005 (mg/g), alpha-Phellandrene 0.0005 (mg/g), gamma-Terpinene 0.002 (mg/g), p-Cymene 0.009 (mg/g), Terpinolene 0.022 (mg/g), Fenchone 0.007 (mg/g), beta-Pinene 1.145 (mg/g), Fenchol 0.0745 (mg/g), Terpineol 0.0155 (mg/g), 3-Carene 0.0025 (mg/g), Myrcene 4.3605 (mg/g), Borneol 0.002 (mg/g), Cedrol 0.002 (mg/g), D-Limonene 2.3125 (mg/g), Caryophyllene oxide 0.0575 (mg/g), BetaCaryophyllene 0.5465 (mg/g), alpha-Farnesene 0.012 (mg/g), beta-Farnesene 0.003 (mg/g), Humulene 0.0965 (mg/g), beta-Ocimene 0.04 (mg/g), alpha-Ocimene 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-psychotic, Anti-emetic, Bronchodilator, Anti-anxiety (Anxiolytic), Appetite Stimulant, Immunosuppressive, Anti-ischemic, Anti-spasmotic

### Formula #25

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; D-Limonene; Caryophyllene oxide; BetaCaryophyllene; Humulene; beta-Ocimene; alpha-Ocimene;

Optionally formulated as: Eucalyptol 0.007 (mg/g), Linalool 0.093 (mg/g), Camphene 0.057 (mg/g), alpha-Pinene 0.577 (mg/g), alpha-Phellandrene 0.119 (mg/g), gamma-Terpinene 0.026 (mg/g), alpha-Terpinene 0.055 (mg/g), p-Cymene 0.098 (mg/g), Terpinolene 2.432 (mg/g), Fenchone 0.01 (mg/g), beta-Pinene 0.908 (mg/g), Fenchol 0.034 (mg/g), Terpineol 0.013 (mg/g), Sabinene 0.01 (mg/g), 3-Carene 0.235 (mg/g), Myrcene 2.233 (mg/g), Borneol 0.001 (mg/g), D-Limonene 1.583 (mg/g), Caryophyllene oxide 0.013 (mg/g), BetaCaryophyllene 0.029 (mg/g), Humulene 0.005 (mg/g), beta-Ocimene 0.643 (mg/g), alpha-Ocimene 0.01 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Immunosuppressive, Anti-psychotic, Anti-emetic, Neuroprotective, Bronchodilator, Anti-ischemic, Anti-anxiety (Anxiolytic), Anti-spasmotic.

### Formula #26

(a) one, two, three, 4, 5, 6, 7, 8, 9, 10 or all of the following terepenes: Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; Humulene; beta-Ocimene; alpha-Ocimene;

Optionally formulated as: Linalool 0.254 (mg/g), Camphene 0.103 (mg/g), alpha-Pinene 0.431 (mg/g), gamma-Terpinene 0.001 (mg/g), p-Cymene 0.005 (mg/g), Terpinolene 0.032 (mg/g), Fenchone 0.021 (mg/g), beta-Pinene 0.807 (mg/g), Fenchol 0.155 (mg/g), Terpineol 0.023 (mg/g), Myrcene 3.163 (mg/g), Borneol 0.004 (mg/g), Cedrol 0.002 (mg/g), D-Limonene 4.253 (mg/g), Caryophyllene oxide 0.043 (mg/g), BetaCaryophyllene 0.524 (mg/g), alpha-Farnesene 0.012 (mg/g), Humulene 0.125 (mg/g), beta-Ocimene 0.033 (mg/g), alpha-Ocimene 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Appetite Stimulant, Bronchodilator, Anti-ischemic, Anti-anxiety (Anxiolytic), Neuroprotective, Anti-emetic, Anti-spasmotic, Immunosuppressive.

### Formula #27

(a) one, two, three, 4, 5, 6, 7, 8, 9, 10 or all of the following terepenes: Menthol; Eucalyptol; Linalool; Camphene; alpha-Pinene; gamma-Terpinene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene;

Optionally formulated as: Menthol 0.002 (mg/g), Eucalyptol 0.008 (mg/g), Linalool 0.271 (mg/g), Camphene 0.023 (mg/g), alpha-Pinene 0.783 (mg/g), gamma-Terpinene 0.002 (mg/g), Terpinolene 0.098 (mg/g), Fenchone 0.004 (mg/g), beta-Pinene 0.403 (mg/g), Fenchol 0.067 (mg/g), Terpineol 0.031 (mg/g), Myrcene 6.471 (mg/g), Borneol 0.004 (mg/g), D-Limonene 0.776 (mg/g), Caryophyllene oxide 0.007 (mg/g), BetaCaryophyllene 0.729 (mg/g), alpha-Farnesene 0.048 (mg/g), beta-Farnesene 0.033 (mg/g), Humulene 0.165 (mg/g), beta-Ocimene 0.071 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-psychotic, Anti-ischemic, Anti-anxiety (Anxiolytic), Anti-emetic, Appetite Stimulant, Neuroprotective, Bronchodilator, Anti-spasmotic

### Formula #28

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; Humulene; beta-Ocimene;

Optionally formulated as: Eucalyptol 0.01 (mg/g), Linalool 0.112 (mg/g), Camphene 0.056 (mg/g), alpha-Pinene 1.868 (mg/g), alpha-Phellandrene 0.001 (mg/g), gamma-Terpinene 0.002 (mg/g), p-Cymene 0.011 (mg/g), Terpinolene 0.041 (mg/g), Fenchone 0.005 (mg/g), beta-Pinene 1.462 (mg/g), Fenchol 0.055 (mg/g), Terpineol 0.009 (mg/g), 3-Carene 0.006 (mg/g), Myrcene 4.821 (mg/g), Borneol 0.002 (mg/g), Cedrol 0.001 (mg/g), D-Limonene 1.119 (mg/g), Caryophyllene oxide 0.031 (mg/g), BetaCaryophyllene 0.254 (mg/g), Humulene 0.048 (mg/g), beta-Ocimene 0.012 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-anxiety (Anxiolytic), Anti-spasmotic, Bronchodilator, Appetite Stimulant, Anti-emetic, Immunosuppressive, Anti-ischemic, Anti-psychotic

### Formula #29

(a) one, two, three, 4, 5, 6, 7, 8, 9, 10 or all of the following terepenes: Eucalyptol; Linalool; Camphene; alpha-Pinene; p-Cymene; Terpinolene; Fenchone; beta-Pinene; Fenchol; Terpineol; Myrcene; Borneol; D-Limonene; Caryophyllene oxide; BetaCaryophyllene; Humulene;

Optionally formulated as: Eucalyptol 0.01 (mg/g), Linalool 0.066 (mg/g), Camphene 0.069 (mg/g), alpha-Pinene 2.551 (mg/g), p-Cymene 0.006 (mg/g), Terpinolene 0.008 (mg/g), Fenchone 0.007 (mg/g), beta-Pinene 1.57 (mg/g), Fenchol 0.035 (mg/g), Terpineol 0.003 (mg/g), Myrcene 3.971 (mg/g), Borneol 0.001 (mg/g), D-Limonene 1.321 (mg/g), Caryophyllene oxide 0.028 (mg/g), BetaCaryophyllene 0.196 (mg/g), Humulene 0.036 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-anxiety (Anxiolytic), Appetite Stimulant, Anti-psychotic, Anti-emetic, Bronchodilator, Neuroprotective, Anti-spasmotic, Immunosuppressive

### Formula #30

(a) one, two, three, 4, 5, 6, 7, 8, 9, 10 or all of the following terepenes: Menthol; Camphor; Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Geranyl Acetate; Caryophyllene oxide; BetaCaryophyllene; alpha-Farnesene; beta-Farnesene; Humulene; beta-Ocimene; alpha-Ocimene; Sabinene Hydrate;

Optionally formulated as: Menthol 0.003 (mg/g), Camphor 0.001 (mg/g), Linalool 0.565 (mg/g), Camphene 0.071 (mg/g), alpha-Pinene 0.244 (mg/g), gamma-Terpinene 0.001 (mg/g), p-Cymene 0.002 (mg/g), Terpinolene 0.027 (mg/g), Fenchone 0.014 (mg/g), beta-Pinene 0.435 (mg/g), Fenchol 0.246 (mg/g), Terpineol 0.084 (mg/g), Myrcene 2.423 (mg/g), Borneol 0.01 (mg/g), Cedrol 0.002 (mg/g), D-Limonene 3.847 (mg/g), Geranyl Acetate 0.001 (mg/g), Caryophyllene oxide 0.048 (mg/g), BetaCaryophyllene 1.449 (mg/g), alpha-Farnesene 0.056 (mg/g), beta-Farnesene 0.016 (mg/g), Humulene 0.318 (mg/g), beta-Ocimene 0.007 (mg/g), alpha-Ocimene 0.008 (mg/g), Sabinene Hydrate 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Bronchodilator, Anti-psychotic, Anti-spasmotic, Anti-emetic, Anti-ischemic, Neuroprotective, Immunosuppressive, Anti-anxiety (Anxiolytic)

### Formula #31

(a) one, two, three, 4, 5, 6, 7, 8, 9, 10 or all of the following terepenes: Eucalyptol; Linalool; Camphene; alpha-Pinene; p-Cymene; Terpinolene; Fenchone; beta-Pinene; Fenchol; Terpineol; Myrcene; Borneol; Cedrol; D-Limonene; Caryophyllene oxide; BetaCaryophyllene; Humulene; beta-Ocimene; alpha-Ocimene;

Optionally formulated as: Eucalyptol 0.023 (mg/g), Linalool 0.093 (mg/g), Camphene 0.025 (mg/g), alpha-Pinene 0.873 (mg/g), p-Cymene 0.008 (mg/g), Terpinolene 0.01 (mg/g), Fenchone 0.003 (mg/g), beta-Pinene 0.36 (mg/g), Fenchol 0.012 (mg/g), Terpineol 0.002 (mg/g), Myrcene 6.868 (mg/g), Borneol 0.001 (mg/g), Cedrol 0.002 (mg/g), D-Limonene 0.669 (mg/g), Caryophyllene oxide 0.069 (mg/g), BetaCaryophyllene 0.314 (mg/g), Humulene 0.05 (mg/g), beta-Ocimene 0.591 (mg/g), alpha-Ocimene 0.007 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-anxiety (Anxiolytic), Appetite Stimulant, Immunosuppressive, Anti-ischemic, Bronchodilator, Anti-emetic, Anti-psychotic, Anti-spasmotic.

### Formula #32

(a) one, two, three, 4, 5, 6, 7, 8, 9, 10 or all of the following terepenes: Linalool; Camphene; alpha-Pinene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene; alpha-Ocimene;

Optionally formulated as: Linalool 0.761 (mg/g), Camphene 0.069 (mg/g), alpha-Pinene 0.234 (mg/g), Terpinolene 0.031 (mg/g), Fenchone 0.015 (mg/g), beta-Pinene 0.402 (mg/g), Fenchol 0.229 (mg/g), Terpineol 0.077 (mg/g), Myrcene 3.663 (mg/g), Borneol 0.012 (mg/g), D-Limonene 2.921 (mg/g), Caryophyllene oxide 0.01 (mg/g), BetaCaryophyllene 1.167 (mg/g), alpha-Farnesene 0.046 (mg/g), beta-Farnesene 0.033 (mg/g), Humulene 0.296 (mg/g), beta-Ocimene 0.007 (mg/g), alpha-Ocimene 0.007 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-emetic, Appetite Stimulant, Anti-spasmotic, Neuroprotective, Bronchodilator, Anti-anxiety (Anxiolytic), Immunosuppressive, Anti-psychotic

### Formula #33

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Guaiol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.027 (mg/g), Linalool 0.308 (mg/g), Camphene 0.011 (mg/g), alpha-Pinene 0.169 (mg/g), alpha-Phellandrene 0.132 (mg/g), gamma-Terpinene 0.045 (mg/g), alpha-Terpinene 0.067 (mg/g), p-Cymene 0.012 (mg/g), Terpinolene 3.921 (mg/g), Fenchone 0.009 (mg/g), beta-Pinene 0.26 (mg/g), Fenchol 0.104 (mg/g), Terpineol 0.11 (mg/g), Sabinene 0.019 (mg/g), 3-Carene 0.1 (mg/g), Myrcene 2.165 (mg/g), Borneol 0.008 (mg/g), Guaiol 0.002 (mg/g), D-Limonene 0.595 (mg/g), Caryophyllene oxide 0.01 (mg/g), BetaCaryophyllene 0.798 (mg/g), alpha-Farnesene 0.049 (mg/g), beta-Farnesene 0.079 (mg/g), Humulene 0.2 (mg/g), beta-Ocimene 0.717 (mg/g), alpha-Ocimene 0.009 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Neuroprotective, Immunosuppressive, Appetite Stimulant, Anti-spasmotic, Anti-psychotic, Bronchodilator, Anti-anxiety (Anxiolytic), Anti-ischemic

### Formula #34

(a) one, two, three, 4, 5, 6, 7, 8, 9, 10 or all of the following terepenes: Eucalyptol; Linalool; Camphene; alpha-Pinene; Pulegone; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.034 (mg/g), Linalool 0.306 (mg/g), Camphene 0.03 (mg/g), alpha-Pinene 0.217 (mg/g), Pulegone 0.005 (mg/g), alpha-Phellandrene 0.012 (mg/g), gamma-Terpinene 0.006 (mg/g), alpha-Terpinene 0.005 (mg/g), p-Cymene 0.044 (mg/g), Terpinolene 0.754 (mg/g), Fenchone 0.015 (mg/g), beta-Pinene 0.402 (mg/g), Fenchol 0.132 (mg/g), Terpineol 0.03 (mg/g), 3-Carene 0.047 (mg/g), Myrcene 1.522 (mg/g), Borneol 0.003 (mg/g), Cedrol 0.009 (mg/g), D-Limonene 2.757 (mg/g), Caryophyllene oxide 0.294 (mg/g), BetaCaryophyllene 1.105 (mg/g), alpha-Farnesene 0.026 (mg/g), beta-Farnesene 0.074 (mg/g), Humulene 0.201 (mg/g), beta-Ocimene 0.028 (mg/g), alpha-Ocimene 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Neuroprotective, Anti-ischemic, Anti-emetic, Appetite Stimulant, Immunosuppressive, Anti-spasmotic, Bronchodilator, Anti-anxiety (Anxiolytic)

### Formula #35

(a) one, two, three, 4, 5, 6, 7, 8, 9, 10 or all of the following terepenes: Eucalyptol;

Linalool; Camphene; alpha-Pinene; p-Cymene; Terpinolene; beta-Pinene; Fenchol; Myrcene; Borneol; D-Limonene; Caryophyllene oxide; BetaCaryophyllene; Humulene; beta-Ocimene;

Optionally formulated as: Eucalyptol 0.041 (mg/g), Linalool 0.164 (mg/g), Camphene 0.02 (mg/g), alpha-Pinene 1.045 (mg/g), p-Cymene 0.006 (mg/g), Terpinolene 0.014 (mg/g), beta-Pinene 0.349 (mg/g), Fenchol 0.006 (mg/g), Myrcene 7.307 (mg/g), Borneol 0.001 (mg/g), D-Limonene 0.391 (mg/g), Caryophyllene oxide 0.021 (mg/g), BetaCaryophyllene 0.151 (mg/g), Humulene 0.025 (mg/g), beta-Ocimene 0.445 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-spasmotic, Anti-ischemic, Appetite Stimulant, Anti-psychotic, Neuroprotective, Anti-anxiety (Anxiolytic), Bronchodilator, Immunosuppressive

### Formula #36

(a) one, two, three, 4, 5, 6, 7, 8, 9, 10 or all of the following terepenes: Eucalyptol; Linalool; Camphene; alpha-Pinene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.038 (mg/g), Linalool 0.065 (mg/g), Camphene 0.052 (mg/g), alpha-Pinene 0.296 (mg/g), gamma-Terpinene 0.001 (mg/g), p-Cymene 0.012 (mg/g), Terpinolene 0.029 (mg/g), Fenchone 0.011 (mg/g), beta-Pinene 0.49 (mg/g), Fenchol 0.111 (mg/g), Terpineol 0.018 (mg/g), 3-Carene 0.004 (mg/g), Myrcene 4.001 (mg/g), Borneol 0.003 (mg/g), Cedrol 0.002 (mg/g), D-Limonene 4.136 (mg/g), Caryophyllene oxide 0.077 (mg/g), BetaCaryophyllene 0.437 (mg/g), alpha-Farnesene 0.012 (mg/g), beta-Farnesene 0.061 (mg/g), Humulene 0.082 (mg/g), beta-Ocimene 0.015 (mg/g), alpha-Ocimene 0.008 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Anti-emetic, Anti-anxiety (Anxiolytic), Anti-ischemic, Appetite Stimulant, Anti-spasmotic, Bronchodilator, Immunosuppressive, Neuroprotective

### Formula #37

(a) one, two, three, 4, 5, 6, 7, 8, 9, 10 or all of the following terepenes: Linalool; Citronellol; Terpinolene; gamma-Terpineol; Fenchone; Fenchol; Borneol; Guaiol; alpha-Bisabolol; BetaCaryophyllene; alpha-Farnesene; beta-Farnesene; Humulene; Sabinene Hydrate; Eucalyptol; Camphene; alpha-Pinene; alpha-Phellandrene; gamma-Terpinene; alpha-Terpinene; p-Cymene; beta-Pinene; Sabinene; Limonene; 3-Carene; Myrcene; beta-Ocimene; trans-beta-Ocimene;

Optionally formulated as: Linalool 2.9 (mg/g), Citronellol 0.1 (mg/g), Terpinolene 217 (mg/g), gamma-Terpineol 0.9 (mg/g), Fenchone 0.5 (mg/g), Fenchol 1.9 (mg/g), Borneol 0.2 (mg/g), Guaiol 0.4 (mg/g), alpha-Bisabolol 0.3 (mg/g), BetaCaryophyllene 20.3 (mg/g), alpha-Farnesene 3.7 (mg/g), beta-Farnesene 14.1 (mg/g), Humulene 6.2 (mg/g), Sabinene Hydrate 0.3 (mg/g), Eucalyptol 3 (mg/g), Camphene 2.6 (mg/g), alpha-Pinene 74.2 (mg/g), alpha-Phellandrene 14.9 (mg/g), gamma-Terpinene 7.3 (mg/g), alpha-Terpinene 8.6 (mg/g), p-Cymene 3.4 (mg/g), beta-Pinene 58.9 (mg/g), Sabinene 0.4 (mg/g), Limonene 81.2 (mg/g), 3-Carene 9.6 (mg/g), Myrcene 377 (mg/g), beta-Ocimene 35.5 (mg/g), trans-beta-Ocimene 2.3 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Anti-emetic, Anti-ischemic, Immunosuppressive, Appetite Stimulant, Anti-psychotic, Bronchodilator, Anti-spasmotic, Neuroprotective, Anti-anxiety (Anxiolytic)

### Formula #38

(a) one, two, three, 4, 5, 6, 7, 8, 9, 10 or all of the following terepenes: Eucalyptol; Linalool; Camphene; alpha-Pinene; p-Cymene; Terpinolene; Fenchone; beta-Pinene; Fenchol; Terpineol; Myrcene; Borneol; Cedrol; D-Limonene; Caryophyllene oxide; BetaCaryophyllene; Humulene; beta-Ocimene; alpha-Ocimene;

Optionally formulated as: Eucalyptol 0.006 (mg/g), Linalool 0.068 (mg/g), Camphene 0.038 (mg/g), alpha-Pinene 1.169 (mg/g), p-Cymene 0.008 (mg/g), Terpinolene 0.011 (mg/g), Fenchone 0.005 (mg/g), beta-Pinene 0.525 (mg/g), Fenchol 0.022 (mg/g), Terpineol 0.005 (mg/g), Myrcene 5.922 (mg/g), Borneol 0.001 (mg/g), Cedrol 0.004 (mg/g), D-Limonene 0.993 (mg/g), Caryophyllene oxide 0.17 (mg/g), BetaCaryophyllene 0.617 (mg/g), Humulene 0.103 (mg/g), beta-Ocimene 0.15 (mg/g), alpha-Ocimene 0.013 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-emetic, Anti-ischemic, Anti-anxiety (Anxiolytic), Immunosuppressive, Bronchodilator, Anti-psychotic, Appetite Stimulant, Anti-spasmotic.

### Formula #39

(a) one, two, three, 4, 5, 6, 7, 8, 9, 10 or all of the following terepenes: Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 0.531 (mg/g), Camphene 0.07 (mg/g), alpha-Pinene 0.391 (mg/g), alpha-Phellandrene 0.005 (mg/g), gamma-Terpinene 0.002 (mg/g), p-Cymene 0.003 (mg/g), Terpinolene 0.126 (mg/g), Fenchone 0.018 (mg/g), beta-Pinene 0.468 (mg/g), Fenchol 0.187 (mg/g), Terpineol 0.084 (mg/g), 3-Carene 0.004 (mg/g), Myrcene 3.129 (mg/g), Borneol 0.009 (mg/g), Cedrol 0.001 (mg/g), D-Limonene 3.512 (mg/g), Caryophyllene oxide 0.017 (mg/g), BetaCaryophyllene 0.933 (mg/g), alpha-Farnesene 0.083 (mg/g), beta-Farnesene 0.06 (mg/g), Humulene 0.213 (mg/g), beta-Ocimene 0.036 (mg/g), alpha-Ocimene 0.009 (mg/g), Sabinene Hydrate 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-emetic, Appetite Stimulant, Bronchodilator, Anti-ischemic, Anti-anxiety (Anxiolytic), Anti-psychotic, Anti-spasmotic, Neuroprotective

### Formula #40

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Cedrol; D-Limonene; Caryophyllene oxide; BetaCaryophyllene; beta-Farnesene; Humulene; beta-Ocimene; alpha-Ocimene; Optionally formulated as: Eucalyptol 0.022 (mg/g), Linalool 0.064 (mg/g), Camphene 0.018 (mg/g), alpha-Pinene 0.741 (mg/g), alpha-Phellandrene 0.117 (mg/g), gamma-Terpinene 0.029 (mg/g), alpha-Terpinene 0.049 (mg/g), p-Cymene 0.051 (mg/g), Terpinolene 2.752 (mg/g), Fenchone 0.001 (mg/g), beta-Pinene 0.7 (mg/g), Fenchol 0.017 (mg/g), Terpineol 0.007 (mg/g), Sabinene 0.011 (mg/g), 3-Carene 0.142 (mg/g), Myrcene 3.946 (mg/g), Borneol 0.001 (mg/g), Cedrol 0.001 (mg/g), D-Limonene 0.544 (mg/g), Caryophyllene oxide 0.018 (mg/g), BetaCaryophyllene 0.172 (mg/g), beta-Farnesene 0.016 (mg/g), Humulene 0.036 (mg/g), beta-Ocimene 0.525 (mg/g), alpha-Ocimene 0.007 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Anti-anxiety (Anxiolytic), Anti-emetic, Immunosuppressive, Neuroprotective, Anti-spasmotic, Anti-ischemic, Bronchodilator, Appetite Stimulant

### Formula #41

(a) one, two, three, 4, 5, 6, 7, 8, 9, 10 or all of the following terepenes: Menthol; Linalool; Camphene; alpha-Pinene; gamma-Terpinene; Terpinolene; Fenchone; beta-Pinene; Fenchol; Terpineol; Myrcene; Borneol; D-Limonene; Caryophyllene oxide; BetaCaryophyllene; alpha-Farnesene; Humulene; Sabinene Hydrate;

Optionally formulated as: Menthol 0.0005 (mg/g), Linalool 0.2585 (mg/g), Camphene 0.02 (mg/g), alpha-Pinene 0.718 (mg/g), gamma-Terpinene 0.0005 (mg/g), Terpinolene 0.013 (mg/g), Fenchone 0.0045 (mg/g), beta-Pinene 0.36 (mg/g), Fenchol 0.0405 (mg/g), Terpineol 0.0155 (mg/g), Myrcene 7.6195 (mg/g), Borneol 0.0025 (mg/g), D-Limonene 0.6195 (mg/g), Caryophyllene oxide 0.0015 (mg/g), BetaCaryophyllene 0.2445 (mg/g), alpha-Farnesene 0.005 (mg/g), Humulene 0.0565 (mg/g), Sabinene Hydrate 0.0015 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Appetite Stimulant, Anti-spasmotic, Bronchodilator, Immunosuppressive, Anti-anxiety (Anxiolytic), Anti-psychotic, Neuroprotective, Anti-ischemic

### Formula #42

(a) one, two, three, 4, 5, 6, 7, 8, 9, 10 or all of the following terepenes: Eucalyptol; Linalool; Camphene; alpha-Pinene; gamma-Terpinene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Eucalyptol 0.007 (mg/g), Linalool 0.682 (mg/g), Camphene 0.073 (mg/g), alpha-Pinene 0.257 (mg/g), gamma-Terpinene 0.002 (mg/g), Terpinolene 0.033 (mg/g), Fenchone 0.015 (mg/g), beta-Pinene 0.457 (mg/g), Fenchol 0.287 (mg/g), Terpineol 0.133 (mg/g), Myrcene 2.045 (mg/g), Borneol 0.012 (mg/g), D-Limonene 3.61 (mg/g), Caryophyllene oxide 0.029 (mg/g), BetaCaryophyllene 1.54 (mg/g), alpha-Farnesene 0.083 (mg/g), beta-Farnesene 0.038 (mg/g), Humulene 0.391 (mg/g), beta-Ocimene 0.006 (mg/g), alpha-Ocimene 0.009 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-ischemic, Anti-psychotic, Anti-emetic, Anti-anxiety (Anxiolytic), Anti-spasmotic, Bronchodilator, Appetite Stimulant, Neuroprotective

### Formula #43

(a) one, two, three, 4, 5, 6, 7, 8, 9, 10 or all of the following terepenes: Camphor; Linalool; Camphene; alpha-Pinene; alpha-Phellandrene; gamma-Terpinene; Terpinolene; Fenchone; beta-Pinene; Fenchol; Terpineol; Sabinene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Camphor 0.001 (mg/g), Linalool 1.113 (mg/g), Camphene 0.055 (mg/g), alpha-Pinene 0.287 (mg/g), alpha-Phellandrene 0.003 (mg/g), gamma-Terpinene 0.003 (mg/g), Terpinolene 0.101 (mg/g), Fenchone 0.03 (mg/g), beta-Pinene 0.386 (mg/g), Fenchol 0.224 (mg/g), Terpineol 0.145 (mg/g), Sabinene 0.001 (mg/g), Myrcene 1.054 (mg/g), Borneol 0.011 (mg/g), Cedrol 0.001 (mg/g), D-Limonene 3.796 (mg/g), Caryophyllene oxide 0.014 (mg/g), BetaCaryophyllene 1.25 (mg/g), alpha-Farnesene 0.122 (mg/g), beta-Farnesene 0.289 (mg/g), Humulene 0.36 (mg/g), beta-Ocimene 0.436 (mg/g), alpha-Ocimene 0.015 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-psychotic, Appetite Stimulant, Bronchodilator, Immunosuppressive, Anti-anxiety (Anxiolytic), Neuroprotective, Anti-ischemic, Anti-emetic

### Formula #44

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; Cedrol; Guaiol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 0.524 (mg/g), Camphene 0.048 (mg/g), alpha-Pinene 0.241 (mg/g), alpha-Phellandrene 0.007 (mg/g), gamma-Terpinene 0.003 (mg/g), alpha-Terpinene 0.003 (mg/g), p-Cymene 0.01 (mg/g), Terpinolene 0.414 (mg/g), Fenchone 0.036 (mg/g), beta-Pinene 0.34 (mg/g), Fenchol 0.134 (mg/g), Terpineol 0.073 (mg/g), 3-Carene 0.008 (mg/g), Myrcene 2.347 (mg/g), Borneol 0.007 (mg/g), Cedrol 0.002 (mg/g), Guaiol 0.003 (mg/g), D-Limonene 4.145 (mg/g), Caryophyllene oxide 0.043 (mg/g), BetaCaryophyllene 0.968 (mg/g), alpha-Farnesene 0.079 (mg/g), beta-Farnesene 0.104 (mg/g), Humulene 0.237 (mg/g), beta-Ocimene 0.021 (mg/g), alpha-Ocimene 0.011 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-emetic, Anti-psychotic, Anti-spasmotic, Anti-anxiety (Anxiolytic), Neuroprotective, Anti-ischemic, Appetite Stimulant, Immunosuppressive

### Formula #45

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; D-Limonene; Caryophyllene oxide; BetaCaryophyllene; alpha-Farnesene; beta-Farnesene; Humulene; beta-Ocimene; Sabinene Hydrate; Optionally formulated as: Linalool 0.14 (mg/g), Camphene 0.034 (mg/g), alpha-Pinene 1.269 (mg/g), alpha-Phellandrene 0.038 (mg/g), gamma-Terpinene 0.004 (mg/g), alpha-Terpinene 0.011 (mg/g), p-Cymene 0.082 (mg/g), Terpinolene 1.174 (mg/g), Fenchone 0.009 (mg/g), beta-Pinene 0.709 (mg/g), Fenchol 0.053 (mg/g), Terpineol 0.035 (mg/g), 3-Carene 0.1 (mg/g), Myrcene 4.062 (mg/g), Borneol 0.003 (mg/g), D-Limonene 1.493 (mg/g), Caryophyllene oxide 0.07 (mg/g), BetaCaryophyllene 0.224 (mg/g), alpha-Farnesene 0.011 (mg/g), beta-Farnesene 0.027 (mg/g), Humulene 0.057 (mg/g), beta-Ocimene 0.02 (mg/g), Sabinene Hydrate 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Bronchodilator, Anti-psychotic, Anti-emetic, Anti-anxiety (Anxiolytic), Anti-ischemic, Appetite Stimulant, Immunosuppressive, Neuroprotective

### Formula #46

(a) one, two, three, 4, 5, 6, 7, 8, 9, 10 or all of the following terepenes: Terpineol; Fenchol; beta-Farnesene; Borneol; Bisabolene; BetaCaryophyllene; alpha-Farnesene; Humulene; cis-Nerolidol; Camphene; alpha-Pinene; Terpinolene; Fenchone; beta-Pinene; Myrcene; D-Limonene; Linalool;

Optionally formulated as: Terpineol 2.3 (mg/g), Fenchol 7.3 (mg/g), beta-Farnesene 2.4 (mg/g), Borneol 1.2 (mg/g), Bisabolene 11.7 (mg/g), BetaCaryophyllene 69 (mg/g), alpha-Farnesene 5.3 (mg/g), Humulene 16.2 (mg/g), cis-Nerolidol 1.2 (mg/g), Camphene 4.6 (mg/g), alpha-Pinene 251.2 (mg/g), Terpinolene 1.5 (mg/g), Fenchone 0.6 (mg/g), beta-Pinene 105.3 (mg/g), Myrcene 270.5 (mg/g), D-Limonene 90.6 (mg/g), Linalool 16.4 (mg/g),

(b) in alternative embodiment, this formulation is used to treat, ameliorate, lesson the symptoms of, or prevent any one of the following conditions, or be used as an: Analgesic, Anti-insomnia, Anti-inflammatory, Anti-bacterial, Anti-cancer, Appetite Suppressant, Anti-depressant, Anti-convulsive,

(c) in alternative embodiment, this formulation is used to treat, ameliorate, lesson the symptoms of, or prevent any one of the following conditions, or be used as an: Bronchodilator, Appetite Stimulant, Neuroprotective, Immunosuppressive, Anti-ischemic, Anti-anxiety (Anxiolytic), Anti-psychotic, Anti-emetic, Anti-spasmotic

### Formula #47

(a) one, two, three, 4, 5, 6, 7, 8, 9, 10 or all of the following terepenes: Terpinolene; alpha-Pinene; alpha-Phellandrene; gamma-Terpinene; alpha-Terpinene; beta-Pinene; Terpineol; Sabinene; beta-Ocimene; Valencene; Linalool; Camphene; p-Cymene; cineole; Camphor; Fenchone; Fenchol; 3-Carene; Myrcene; Borneol; D-Limonene; Caryophyllene oxide; Bisabolene; BetaCaryophyllene; alpha-Farnesene; beta-Farnesene; Humulene; Nerolidol; cis-Nerolidol; alpha-Ocimene; beta-Elemene;

Optionally formulated as: Terpinolene 46.3 (mg/g), alpha-Pinene 17.4 (mg/g), alpha-Phellandrene 2.7 (mg/g), gamma-Terpinene 0.4 (mg/g), alpha-Terpinene 0.7 (mg/g), beta-Pinene 27.7 (mg/g), Terpineol 3.4 (mg/g), Sabinene 0.3 (mg/g), beta-Ocimene 8.3 (mg/g), Valencene 3.8 (mg/g), Linalool 17.2 (mg/g), Camphene 3.5 (mg/g), p-Cymene 1.1 (mg/g), cineole 0.2 (mg/g), Camphor 0.5 (mg/g), Fenchone 1.5 (mg/g), Fenchol 8.2 (mg/g), 3-Carene 2.6 (mg/g), Myrcene 147 (mg/g), Borneol 0.9 (mg/g), D-Limonene 216.6 (mg/g), Caryophyllene oxide 3.2 (mg/g), Bisabolene 17.5 (mg/g), BetaCaryophyllene 70.9 (mg/g), alpha-Farnesene 2.8 (mg/g), beta-Farnesene 9.3 (mg/g), Humulene 16.3 (mg/g), Nerolidol 0.2 (mg/g), cis-Nerolidol 1.2 (mg/g), alpha-Ocimene 0.3 (mg/g), beta-Elemene 0.6 (mg/g),

(b) in alternative embodiment, this formulation is used to treat, ameliorate, lesson the symptoms of, or prevent any one of the following conditions, or be used as an: Anti-bacterial, Anti-insomnia, Anti-inflammatory, Anti-depressant, Anti-convulsive, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-emetic, Anti-spasmotic, Anti-psychotic, Neuroprotective, Anti-anxiety (Anxiolytic), Bronchodilator, Immunosuppressive, Anti-ischemic

### Formula #48

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 1.074 (mg/g), Camphene 0.087 (mg/g), alpha-Pinene 0.448 (mg/g), p-Cymene 0.002 (mg/g), Terpinolene 0.025 (mg/g), Fenchone 0.012 (mg/g), beta-Pinene 0.554 (mg/g), Fenchol 0.251 (mg/g), Terpineol 0.102 (mg/g), Myrcene 1.152 (mg/g), Borneol 0.008 (mg/g), D-Limonene 4.662 (mg/g), Caryophyllene oxide 0.039 (mg/g), BetaCaryophyllene 0.921 (mg/g), alpha-Farnesene 0.036 (mg/g), beta-Farnesene 0.019 (mg/g), Humulene 0.237 (mg/g), beta-Ocimene 0.083 (mg/g), alpha-Ocimene 0.009 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Bronchodilator, Immunosuppressive, Anti-ischemic, Anti-spasmotic, Anti-anxiety (Anxiolytic), Appetite Stimulant, Anti-emetic, Neuroprotective

### Formula #49

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; Myrcene; Borneol; Cedrol; Isopulegol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 1.063 (mg/g), Camphene 0.088 (mg/g), alpha-Pinene 0.45 (mg/g), p-Cymene 0.002 (mg/g), Terpinolene 0.0245 (mg/g), Fenchone 0.012 (mg/g), beta-Pinene 0.5565 (mg/g), Fenchol 0.2525 (mg/g), Terpineol 0.1005 (mg/g), Myrcene 1.1545 (mg/g), Borneol 0.008 (mg/g), Cedrol 0.001 (mg/g), Isopulegol 0.0095 (mg/g), D-Limonene 4.7165 (mg/g), Caryophyllene oxide 0.039 (mg/g), BetaCaryophyllene 0.911 (mg/g), alpha-Farnesene 0.033 (mg/g), beta-Farnesene 0.0175 (mg/g), Humulene 0.2345 (mg/g), beta-Ocimene 0.083 (mg/g), alpha-Ocimene 0.0095 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-psychotic, Anti-emetic, Immunosuppressive, Anti-ischemic, Appetite Stimulant, Anti-anxiety (Anxiolytic), Neuroprotective, Bronchodilator

### Formula #50

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; Humulene; beta-Ocimene;

Optionally formulated as: Linalool 0.634 (mg/g), Camphene 0.069 (mg/g), alpha-Pinene 0.23 (mg/g), p-Cymene 0.009 (mg/g), Terpinolene 0.007 (mg/g), Fenchone 0.015 (mg/g), beta-Pinene 0.556 (mg/g), Fenchol 0.261 (mg/g), Terpineol 0.053 (mg/g), 3-Carene 0.004 (mg/g), Myrcene 1.198 (mg/g), Borneol 0.006 (mg/g), Cedrol 0.013 (mg/g), D-Limonene 4.996 (mg/g), Caryophyllene oxide 0.399 (mg/g), BetaCaryophyllene 0.925 (mg/g), alpha-Farnesene 0.031 (mg/g), Humulene 0.232 (mg/g), beta-Ocimene 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Bronchodilator, Anti-ischemic, Neuroprotective, Anti-spasmotic, Anti-psychotic, Anti-emetic, Appetite Stimulant, Anti-anxiety (Anxiolytic)

### Formula #51

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.015 (mg/g), Linalool 0.113 (mg/g), Camphene 0.084 (mg/g), alpha-Pinene 1.403 (mg/g), alpha-Phellandrene 0.006 (mg/g), gamma-Terpinene 0.003 (mg/g), p-Cymene 0.031 (mg/g), Terpinolene 0.184 (mg/g), Fenchone 0.006 (mg/g), beta-Pinene 1.374 (mg/g), Fenchol 0.078 (mg/g), Terpineol 0.012 (mg/g), 3-Carene 0.034 (mg/g), Myrcene 2.208 (mg/g), Borneol 0.002 (mg/g), Cedrol 0.002 (mg/g), D-Limonene 3.682 (mg/g), Caryophyllene oxide 0.089 (mg/g), BetaCaryophyllene 0.391 (mg/g), alpha-Farnesene 0.014 (mg/g), beta-Farnesene 0.023 (mg/g), Humulene 0.071 (mg/g), beta-Ocimene 0.048 (mg/g), alpha-Ocimene 0.008 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Neuroprotective, Anti-emetic, Appetite Stimulant, Anti-spasmotic, Bronchodilator, Anti-ischemic, Anti-anxiety (Anxiolytic), Anti-psychotic

### Formula #52

(a) one, two, three, 4, 5, 6, 7, 8, 9, 10 or all of the following terepenes: Menthol; Eucalyptol; Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Cedrol; D-Limonene; Caryophyllene oxide; BetaCaryophyllene; beta-Farnesene; Humulene; beta-Ocimene; alpha-Ocimene; Sabinene Hydrate;

Optionally formulated as: Menthol 0.0015 (mg/g), Eucalyptol 0.0225 (mg/g), Linalool 0.0665 (mg/g), Camphene 0.0215 (mg/g), alpha-Pinene 0.742 (mg/g), alpha-Phellandrene 0.1095 (mg/g), gamma-Terpinene 0.0345 (mg/g), alpha-Terpinene 0.0475 (mg/g), p-Cymene 0.05 (mg/g), Terpinolene 2.751 (mg/g), Fenchone 0.0005 (mg/g), beta-Pinene 0.6995 (mg/g), Fenchol 0.017 (mg/g), Terpineol 0.0065 (mg/g), Sabinene 0.0055 (mg/g), 3-Carene 0.137 (mg/g), Myrcene 3.9535 (mg/g), Borneol 0.0005 (mg/g), Cedrol 0.0005 (mg/g), D-Limonene 0.5445 (mg/g), Caryophyllene oxide 0.018 (mg/g), BetaCaryophyllene 0.174 (mg/g), beta-Farnesene 0.0095 (mg/g), Humulene 0.037 (mg/g), beta-Ocimene 0.526 (mg/g), alpha-Ocimene 0.0065 (mg/g), Sabinene Hydrate 0.0015 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Immunosuppressive, Anti-psychotic, Anti-emetic, Bronchodilator, Anti-spasmotic, Anti-ischemic, Neuroprotective, Anti-anxiety (Anxiolytic)

### Formula #53

(a) one, two, three, 4, 5, 6, 7, 8, 9, 10 or all of the following terepenes: Linalool; Camphene; alpha-Pinene; gamma-Terpinene; p-Cymene; Terpinolene; Fenchone; beta-Pinene; Fenchol; Terpineol; Myrcene; Borneol; D-Limonene; Caryophyllene oxide; BetaCaryophyllene; alpha-Farnesene; Humulene; beta-Ocimene; alpha-Ocimene;

Optionally formulated as: Linalool 0.162 (mg/g), Camphene 0.071 (mg/g), alpha-Pinene 0.186 (mg/g), gamma-Terpinene 0.002 (mg/g), p-Cymene 0.004 (mg/g), Terpinolene 0.034 (mg/g), Fenchone 0.018 (mg/g), beta-Pinene 0.556 (mg/g), Fenchol 0.134 (mg/g), Terpineol 0.018 (mg/g), Myrcene 2.346 (mg/g), Borneol 0.003 (mg/g), D-Limonene 6.14 (mg/g), Caryophyllene oxide 0.017 (mg/g), BetaCaryophyllene 0.234 (mg/g), alpha-Farnesene 0.004 (mg/g), Humulene 0.04 (mg/g), beta-Ocimene 0.003 (mg/g), alpha-Ocimene 0.011 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Immunosuppressive, Anti-psychotic, Anti-anxiety (Anxiolytic), Anti-emetic, Anti-ischemic, Bronchodilator, Neuroprotective, Anti-spasmotic.

### Formula #54

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; Myrcene; Bomeol; Cedrol; Isopulegol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 1.052 (mg/g), Camphene 0.089 (mg/g), alpha-Pinene 0.452 (mg/g), p-Cymene 0.002 (mg/g), Terpinolene 0.024 (mg/g), Fenchone 0.012 (mg/g), beta-Pinene 0.559 (mg/g), Fenchol 0.254 (mg/g), Terpineol 0.099 (mg/g), Myrcene 1.157 (mg/g), Borneol 0.008 (mg/g), Cedrol 0.002 (mg/g), Isopulegol 0.019 (mg/g), D-Limonene 4.771 (mg/g), Caryophyllene oxide 0.039 (mg/g), BetaCaryophyllene 0.901 (mg/g), alpha-Farnesene 0.03 (mg/g), beta-Farnesene 0.016 (mg/g), Humulene 0.232 (mg/g), beta-Ocimene 0.083 (mg/g), alpha-Ocimene 0.01 (mg/g), Sabinene Hydrate 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-ischemic, Appetite Stimulant, Anti-anxiety (Anxiolytic), Neuroprotective, Anti-spasmotic, Anti-emetic, Anti-psychotic, Bronchodilator

### Formula #55

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene; cis-beta-ocimene;

Optionally formulated as: Eucalyptol 0.007 (mg/g), Linalool 0.371 (mg/g), Camphene 0.099 (mg/g), alpha-Pinene 0.346 (mg/g), alpha-Phellandrene 0.002 (mg/g), gamma-Terpinene 0.003 (mg/g), p-Cymene 0.002 (mg/g), Terpinolene 0.061 (mg/g), Fenchone 0.01 (mg/g), beta-Pinene 0.86 (mg/g), Fenchol 0.126 (mg/g), Terpineol 0.033 (mg/g), Myrcene 1.781 (mg/g), Borneol 0.008 (mg/g), D-Limonene 4.516 (mg/g), Caryophyllene oxide 0.01 (mg/g), BetaCaryophyllene 0.644 (mg/g), alpha-Farnesene 0.137 (mg/g), beta-Farnesene 0.106 (mg/g), Humulene 0.144 (mg/g), beta-Ocimene 0.013 (mg/g), cis-beta-ocimene 0.023 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Anti-spasmotic, Appetite Stimulant, Immunosuppressive, Neuroprotective, Anti-emetic, Anti-ischemic, Bronchodilator, Anti-anxiety (Anxiolytic)

### Formula #56

(a) one, two, three, 4, 5, 6, 7, 8, 9, 10 or all of the following terepenes: Eucalyptol; Linalool; Camphene; alpha-Pinene; Pulegone; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.027 (mg/g), Linalool 0.023 (mg/g), Camphene 0.043 (mg/g), alpha-Pinene 0.608 (mg/g), Pulegone 0.003 (mg/g), alpha-Phellandrene 0.061 (mg/g), gamma-Terpinene 0.022 (mg/g), alpha-Terpinene 0.029 (mg/g), p-Cymene 0.044 (mg/g), Terpinolene 2.092 (mg/g), Fenchone 0.007 (mg/g), beta-Pinene 0.648 (mg/g), Fenchol 0.066 (mg/g), Terpineol 0.012 (mg/g), 3-Carene 0.101 (mg/g), Myrcene 1.556 (mg/g), Borneol 0.002 (mg/g), Cedrol 0.002 (mg/g), D-Limonene 2.803 (mg/g), Caryophyllene oxide 0.078 (mg/g), BetaCaryophyllene 0.817 (mg/g), alpha-Farnesene 0.082 (mg/g), beta-Farnesene 0.018 (mg/g), Humulene 0.124 (mg/g), beta-Ocimene 0.545 (mg/g), alpha-Ocimene 0.025 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Bronchodilator, Anti-emetic, Neuroprotective, Anti-spasmotic, Immunosuppressive, Anti-ischemic, Appetite Stimulant, Anti-psychotic

### Formula #57

(a) one, two, three, 4, 5, 6, 7, 8, 9, 10 or all of the following terepenes: Eucalyptol; Linalool; Camphene; alpha-Pinene; Pulegone; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.026 (mg/g), Linalool 0.024 (mg/g), Camphene 0.044 (mg/g), alpha-Pinene 0.612 (mg/g), Pulegone 0.002 (mg/g), alpha-Phellandrene 0.062 (mg/g), gamma-Terpinene 0.023 (mg/g), alpha-Terpinene 0.031 (mg/g), p-Cymene 0.046 (mg/g), Terpinolene 2.122 (mg/g), Fenchone 0.007 (mg/g), beta-Pinene 0.655 (mg/g), Fenchol 0.066 (mg/g), Terpineol 0.013 (mg/g), Sabinene 0.006 (mg/g), 3-Carene 0.103 (mg/g), Myrcene 1.593 (mg/g), Borneol 0.002 (mg/g), Cedrol 0.002 (mg/g), D-Limonene 2.769 (mg/g), Caryophyllene oxide 0.078 (mg/g), BetaCaryophyllene 0.82 (mg/g), alpha-Farnesene 0.011 (mg/g), beta-Farnesene 0.009 (mg/g), Humulene 0.126 (mg/g), beta-Ocimene 0.552 (mg/g), alpha-Ocimene 0.026 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-anxiety (Anxiolytic), Anti-ischemic, Immunosuppressive, Bronchodilator, Anti-spasmotic, Appetite Stimulant, Anti-emetic, Anti-psychotic

### Formula #58

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.011 (mg/g), Linalool 0.071 (mg/g), Camphene 0.043 (mg/g), alpha-Pinene 0.571 (mg/g), alpha-Phellandrene 0.054 (mg/g), gamma-Terpinene 0.021 (mg/g), alpha-Terpinene 0.026 (mg/g), p-Cymene 0.017 (mg/g), Terpinolene 1.508 (mg/g), Fenchone 0.008 (mg/g), beta-Pinene 0.716 (mg/g), Fenchol 0.051 (mg/g), Terpineol 0.01 (mg/g), Sabinene 0.006 (mg/g), 3-Carene 0.068 (mg/g), Myrcene 4.175 (mg/g), Borneol 0.001 (mg/g), Cedrol 0.001 (mg/g), D-Limonene 2.062 (mg/g), Caryophyllene oxide 0.018 (mg/g), BetaCaryophyllene 0.43 (mg/g), alpha-Farnesene 0.005 (mg/g), beta-Farnesene 0.015 (mg/g), Humulene 0.075 (mg/g), beta-Ocimene 0.027 (mg/g), alpha-Ocimene 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Anti-psychotic, Neuroprotective, Immunosuppressive, Anti-ischemic, Bronchodilator, Anti-spasmotic, Anti-emetic, Appetite Stimulant

### Formula #59

(a) one, two, three, 4, 5, 6, 7, 8, 9, 10 or all of the following terepenes: Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; D-Limonene; Caryophyllene oxide; BetaCaryophyllene; Humulene; Valencene;

Optionally formulated as: Linalool 0.018 (mg/g), Camphene 0.047 (mg/g), alpha-Pinene 1.08 (mg/g), alpha-Phellandrene 0.002 (mg/g), gamma-Terpinene 0.001 (mg/g), p-Cymene 0.003 (mg/g), Terpinolene 0.042 (mg/g), Fenchone 0.003 (mg/g), beta-Pinene 0.755 (mg/g), Fenchol 0.034 (mg/g), Terpineol 0.004 (mg/g), 3-Carene 0.003 (mg/g), Myrcene 6.594 (mg/g), Borneol 0.001 (mg/g), D-Limonene 1.167 (mg/g), Caryophyllene oxide 0.003 (mg/g), BetaCaryophyllene 0.074 (mg/g), Humulene 0.012 (mg/g), Valencene 0.002 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Anti-spasmotic, Appetite Stimulant, Bronchodilator, Neuroprotective, Anti-ischemic, Anti-emetic, Immunosuppressive, Anti-psychotic

### Formula #60

(a) one, two, three, 4, 5, 6, 7, 8, 9, 10 or all of the following terepenes: Linalool; Camphene; alpha-Pinene; alpha-Phellandrene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene;

Optionally formulated as: Linalool 0.061 (mg/g), Camphene 0.037 (mg/g), alpha-Pinene 0.615 (mg/g), alpha-Phellandrene 0.004 (mg/g), p-Cymene 0.016 (mg/g), Terpinolene 0.208 (mg/g), Fenchone 0.007 (mg/g), beta-Pinene 0.573 (mg/g), Fenchol 0.063 (mg/g), Terpineol 0.012 (mg/g), 3-Carene 0.018 (mg/g), Myrcene 5.906 (mg/g), Borneol 0.002 (mg/g), Cedrol 0.003 (mg/g), D-Limonene 1.602 (mg/g), Caryophyllene oxide 0.083 (mg/g), BetaCaryophyllene 0.392 (mg/g), alpha-Farnesene 0.011 (mg/g), beta-Farnesene 0.013 (mg/g), Humulene 0.072 (mg/g), beta-Ocimene 0.158 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Bronchodilator, Anti-spasmotic, Appetite Stimulant, Anti-anxiety (Anxiolytic), Anti-emetic, Neuroprotective, Anti-psychotic, Anti-ischemic

### Formula #61

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; beta-Farnesene; Humulene; beta-Ocimene; alpha-Ocimene; Sabinene Hydrate;

Optionally formulated as: Linalool 0.631 (mg/g), Camphene 0.06 (mg/g), alpha-Pinene 0.226 (mg/g), p-Cymene 0.003 (mg/g), Terpinolene 0.022 (mg/g), Fenchone 0.014 (mg/g), beta-Pinene 0.375 (mg/g), Fenchol 0.203 (mg/g), Terpineol 0.083 (mg/g), Myrcene 2.344 (mg/g), Borneol 0.008 (mg/g), Cedrol 0.002 (mg/g), D-Limonene 3.916 (mg/g), Caryophyllene oxide 0.065 (mg/g), BetaCaryophyllene 1.233 (mg/g), alpha-Farnesene 0.055 (mg/g), beta-Farnesene 0.089 (mg/g), Humulene 0.345 (mg/g), beta-Ocimene 0.004 (mg/g), alpha-Ocimene 0.005 (mg/g), Sabinene Hydrate 0.004 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Appetite Stimulant, Anti-spasmotic, Anti-psychotic, Neuroprotective, Anti-ischemic, Anti-emetic, Immunosuppressive, Anti-anxiety (Anxiolytic)

### Formula #62

(a) one, two, three, 4, 5, 6, 7, 8, 9, 10 or all of the following terepenes: Eucalyptol; Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; beta-Farnesene; Humulene; beta-Ocimene; Sabinene Hydrate;

Optionally formulated as: Eucalyptol 0.05 (mg/g), Linalool 0.085 (mg/g), Camphene 0.026 (mg/g), alpha-Pinene 0.59 (mg/g), gamma-Terpinene 0.003 (mg/g), p-Cymene 0.012 (mg/g), Terpinolene 0.018 (mg/g), Fenchone 0.005 (mg/g), beta-Pinene 0.446 (mg/g), Fenchol 0.02 (mg/g), Terpineol 0.003 (mg/g), Myrcene 6.5 (mg/g), Borneol 0.001 (mg/g), Cedrol 0.003 (mg/g), D-Limonene 0.785 (mg/g), Caryophyllene oxide 0.079 (mg/g), BetaCaryophyllene 0.697 (mg/g), alpha-Farnesene 0.02 (mg/g), beta-Farnesene 0.056 (mg/g), Humulene 0.162 (mg/g), beta-Ocimene 0.356 (mg/g), Sabinene Hydrate 0.002 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-anxiety (Anxiolytic), Anti-spasmotic, Appetite Stimulant, Anti-emetic, Anti-psychotic, Bronchodilator, Anti-ischemic, Immunosuppressive

### Formula #63

(a) one, two, three, 4, 5, 6, 7, 8, 9, 10 or all of the following terepenes: Eucalyptol; Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; Humulene; beta-Ocimene;

Optionally formulated as: Eucalyptol 0.036 (mg/g), Linalool 0.067 (mg/g), Camphene 0.022 (mg/g), alpha-Pinene 0.745 (mg/g), alpha-Phellandrene 0.001 (mg/g), gamma-Terpinene 0.004 (mg/g), p-Cymene 0.012 (mg/g), Terpinolene 0.07 (mg/g), Fenchone 0.008 (mg/g), beta-Pinene 0.49 (mg/g), Fenchol 0.034 (mg/g), Terpineol 0.005 (mg/g), 3-Carene 0.005 (mg/g), Myrcene 6.586 (mg/g), Borneol 0.002 (mg/g), Cedrol 0.001 (mg/g), D-Limonene 1.208 (mg/g), Caryophyllene oxide 0.037 (mg/g), BetaCaryophyllene 0.477 (mg/g), Humulene 0.08 (mg/g), beta-Ocimene 0.021 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-spasmotic, Immunosuppressive, Appetite Stimulant, Bronchodilator, Anti-psychotic, Anti-ischemic, Anti-emetic, Anti-anxiety (Anxiolytic)

### Formula #64

(a) one, two, three, 4, 5, 6, 7, 8, 9, 10 or all of the following terepenes: Eucalyptol; Linalool; Camphene; alpha-Pinene; p-Cymene; Terpinolene; beta-Pinene; Fenchol; Myrcene; Borneol; D-Limonene; Caryophyllene oxide; BetaCaryophyllene; Humulene; beta-Ocimene;

Optionally formulated as: Eucalyptol 0.0405 (mg/g), Linalool 0.166 (mg/g), Camphene 0.021 (mg/g), alpha-Pinene 1.052 (mg/g), p-Cymene 0.006 (mg/g), Terpinolene 0.0135 (mg/g), beta-Pinene 0.351 (mg/g), Fenchol 0.0065 (mg/g), Myrcene 7.306 (mg/g), Borneol 0.001 (mg/g), D-Limonene 0.3875 (mg/g), Caryophyllene oxide 0.0205 (mg/g), BetaCaryophyllene 0.1515 (mg/g), Humulene 0.0245 (mg/g), beta-Ocimene 0.443 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Bronchodilator, Anti-emetic, Neuroprotective, Anti-anxiety (Anxiolytic), Appetite Stimulant, Immunosuppressive, Anti-ischemic, Anti-spasmotic.

### Formula #65

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; Terpinolene; Fenchone; beta-Pinene; Fenchol; Terpineol; 3-Carene; Myrcene; Borneol; Alloaromadendrene; Guaiol; D-Limonene; Nerol; alpha-Bisabolol; Caryophyllene oxide; BetaCaryophyllene; alpha-Farnesene; beta-Farnesene; Humulene; beta-Ocimene; alpha-Ocimene; Sabinene Hydrate;

Optionally formulated as: Eucalyptol 0.025 (mg/g), Linalool 0.749 (mg/g), Camphene 0.023 (mg/g), alpha-Pinene 0.277 (mg/g), alpha-Phellandrene 0.002 (mg/g), gamma-Terpinene 0.003 (mg/g), alpha-Terpinene 0.002 (mg/g), Terpinolene 0.052 (mg/g), Fenchone 0.02 (mg/g), beta-Pinene 0.225 (mg/g), Fenchol 0.158 (mg/g), Terpineol 0.094 (mg/g), 3-Carene 0.001 (mg/g), Myrcene 2.414 (mg/g), Borneol 0.013 (mg/g), Alloaromadendrene 0.067 (mg/g), Guaiol 0.013 (mg/g), D-Limonene 0.751 (mg/g), Nerol 0.002 (mg/g), alpha-Bisabolol 0.009 (mg/g), Caryophyllene oxide 0.011 (mg/g), BetaCaryophyllene 2.495 (mg/g), alpha-Farnesene 0.173 (mg/g), beta-Farnesene 0.317 (mg/g), Humulene 0.558 (mg/g), beta-Ocimene 0.22 (mg/g), alpha-Ocimene 0.004 (mg/g), Sabinene Hydrate 0.008 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Bronchodilator, Anti-spasmotic, Anti-psychotic, Neuroprotective, Appetite Stimulant, Anti-emetic, Anti-ischemic, Immunosuppressive

### Formula #66

(a) one, two, three, 4, 5, 6, 7, 8, 9, 10 or all of the following terepenes: Menthol; Eucalyptol; Linalool; Camphene; alpha-Pinene; alpha-Phellandrene; gamma-Terpinene; alpha-Terpinene; p-Cymene; Terpinolene; beta-Pinene; Fenchol; Terpineol; 3-Carene; Myrcene; D-Limonene; Caryophyllene oxide; BetaCaryophyllene; beta-Farnesene; Humulene; beta-Ocimene; alpha-Ocimene; Sabinene Hydrate;

Optionally formulated as: Menthol 0.003 (mg/g), Eucalyptol 0.023 (mg/g), Linalool 0.069 (mg/g), Camphene 0.025 (mg/g), alpha-Pinene 0.743 (mg/g), alpha-Phellandrene 0.102 (mg/g), gamma-Terpinene 0.04 (mg/g), alpha-Terpinene 0.046 (mg/g), p-Cymene 0.049 (mg/g), Terpinolene 2.75 (mg/g), beta-Pinene 0.699 (mg/g), Fenchol 0.017 (mg/g), Terpineol 0.006 (mg/g), 3-Carene 0.132 (mg/g), Myrcene 3.961 (mg/g), D-Limonene 0.545 (mg/g), Caryophyllene oxide 0.018 (mg/g), BetaCaryophyllene 0.176 (mg/g), beta-Farnesene 0.003 (mg/g), Humulene 0.038 (mg/g), beta-Ocimene 0.527 (mg/g), alpha-Ocimene 0.006 (mg/g), Sabinene Hydrate 0.003 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Immunosuppressive, Anti-emetic, Anti-anxiety (Anxiolytic), Appetite Stimulant, Bronchodilator, Anti-ischemic, Anti-psychotic, Anti-spasmotic.

### Formula #67

(a) one, two, three, 4, 5, 6, 7, 8, 9, 10 or all of the following terepenes: Eucalyptol; Linalool; Camphene; alpha-Pinene; Pulegone; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.0265 (mg/g), Linalool 0.0235 (mg/g), Camphene 0.0435 (mg/g), alpha-Pinene 0.61 (mg/g), Pulegone 0.0025 (mg/g), alpha-Phellandrene 0.0615 (mg/g), gamma-Terpinene 0.0225 (mg/g), alpha-Terpinene 0.03 (mg/g), p-Cymene 0.045 (mg/g), Terpinolene 2.107 (mg/g), Fenchone 0.007 (mg/g), beta-Pinene 0.6515 (mg/g), Fenchol 0.066 (mg/g), Terpineol 0.0125 (mg/g), Sabinene 0.003 (mg/g), 3-Carene 0.102 (mg/g), Myrcene 1.5745 (mg/g), Borneol 0.002 (mg/g), Cedrol 0.002 (mg/g), D-Limonene 2.786 (mg/g), Caryophyllene oxide 0.078 (mg/g), BetaCaryophyllene 0.8185 (mg/g), alpha-Farnesene 0.0465 (mg/g), beta-Farnesene 0.0135 (mg/g), Humulene 0.125 (mg/g), beta-Ocimene 0.5485 (mg/g), alpha-Ocimene 0.0255 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-ischemic, Neuroprotective, Anti-anxiety (Anxiolytic), Immunosuppressive, Anti-emetic, Bronchodilator, Anti-psychotic, Appetite Stimulant

### Formula #68

(a) one, two, three, 4, 5, 6, 7, 8, 9, 10 or all of the following terepenes: Camphor; Linalool; BetaCaryophyllene; Fenchol; Terpineol; Borneol; Caryophyllene oxide; Bisabolene; alpha-Farnesene; beta-Farnesene; Humulene; Nerolidol; cis-Nerolidol; Valencene; Eucalyptol; Camphene; alpha-Pinene; cineole; Terpinolene; Fenchone; Myrcene; D-Limonene; beta-Pinene; beta-Ocimene; alpha-Ocimene; Sabinene Hydrate;

Optionally formulated as: Camphor 0.2 (mg/g), Linalool 143.9 (mg/g), BetaCaryophyllene 144.3 (mg/g), Fenchol 12.2 (mg/g), Terpineol 3.8 (mg/g), Borneol 1.7 (mg/g), Caryophyllene oxide 0.9 (mg/g), Bisabolene 11.8 (mg/g), alpha-Farnesene 1.3 (mg/g), beta-Farnesene 1.3 (mg/g), Humulene 36.1 (mg/g), Nerolidol 0.4 (mg/g), cis-Nerolidol 1.5 (mg/g), Valencene 5 (mg/g), Eucalyptol 0.4 (mg/g), Camphene 5.3 (mg/g), alpha-Pinene 20.1 (mg/g), cineole 0.9 (mg/g), Terpinolene 2.2 (mg/g), Fenchone 1.4 (mg/g), Myrcene 124.2 (mg/g), D-Limonene 177 (mg/g), beta-Pinene 33.5 (mg/g), beta-Ocimene 1 (mg/g), alpha-Ocimene 0.5 (mg/g), Sabinene Hydrate 0.3 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Anti-ischemic, Anti-spasmotic, Neuroprotective, Appetite Stimulant, Immunosuppressive, Anti-emetic, Anti-anxiety (Anxiolytic), Anti-psychotic

### Formula #69

(a) one, two, three, 4, 5, 6, 7, 8, 9, 10 or all of the following terepenes: Camphor; Linalool; BetaCaryophyllene; Fenchol; Terpineol; Borneol; Caryophyllene oxide; Bisabolene; alpha-Farnesene; beta-Farnesene; Humulene; Nerolidol; cis-Nerolidol; Valencene; Eucalyptol; Camphene; alpha-Pinene; cineole; Terpinolene; Fenchone; Myrcene; D-Limonene; beta-Pinene; beta-Ocimene; alpha-Ocimene; Sabinene Hydrate;

Optionally formulated as: Camphor 0.1 (mg/g), Linalool 144 (mg/g), BetaCaryophyllene 145 (mg/g), Fenchol 12 (mg/g), Terpineol 4 (mg/g), Borneol 1 (mg/g), Caryophyllene oxide 1 (mg/g), Bisabolene 12 (mg/g), alpha-Farnesene 2 (mg/g), beta-Farnesene 1 (mg/g), Humulene 35 (mg/g), Nerolidol 0.3 (mg/g), cis-Nerolidol 1.8 (mg/g), Valencene 4 (mg/g), Eucalyptol 0.1 (mg/g), Camphene 5.9 (mg/g), alpha-Pinene 20 (mg/g), cineole 0.9 (mg/g), Terpinolene 2 (mg/g), Fenchone 1.2 (mg/g), Myrcene 110 (mg/g), D-Limonene 179 (mg/g), beta-Pinene 35 (mg/g), beta-Ocimene 1 (mg/g), alpha-Ocimene 1 (mg/g), Sabinene Hydrate 0.9 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Anti-ischemic, Anti-spasmotic, Neuroprotective, Appetite Stimulant, Immunosuppressive, Anti-emetic, Anti-anxiety (Anxiolytic), Anti-psychotic

### Formula #70

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; beta-Farnesene; Humulene; beta-Ocimene; Sabinene Hydrate;

Optionally formulated as: Linalool 0.111 (mg/g), Camphene 0.014 (mg/g), alpha-Pinene 0.879 (mg/g), p-Cymene 0.001 (mg/g), Terpinolene 0.006 (mg/g), Fenchone 0.002 (mg/g), beta-Pinene 0.26 (mg/g), Fenchol 0.032 (mg/g), Terpineol 0.014 (mg/g), Myrcene 6.84 (mg/g), Borneol 0.003 (mg/g), Cedrol 0.003 (mg/g), D-Limonene 0.541 (mg/g), Caryophyllene oxide 0.064 (mg/g), BetaCaryophyllene 0.617 (mg/g), alpha-Farnesene 0.022 (mg/g), beta-Farnesene 0.011 (mg/g), Humulene 0.129 (mg/g), beta-Ocimene 0.384 (mg/g), Sabinene Hydrate 0.003 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-ischemic, Immunosuppressive, Neuroprotective, Bronchodilator, Anti-psychotic, Anti-spasmotic, Anti-anxiety (Anxiolytic), Anti-emetic

### Formula #71

(a) one, two, three, 4, 5, 6, 7, 8, 9, 10 or all of the following terepenes: Menthol; Eucalyptol; Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Menthol 0.001 (mg/g), Eucalyptol 0.024 (mg/g), Linalool 0.063 (mg/g), Camphene 0.04 (mg/g), alpha-Pinene 0.34 (mg/g), gamma-Terpinene 0.002 (mg/g), p-Cymene 0.008 (mg/g), Terpinolene 0.051 (mg/g), Fenchone 0.005 (mg/g), beta-Pinene 0.468 (mg/g), Fenchol 0.052 (mg/g), Terpineol 0.005 (mg/g), Myrcene 4.866 (mg/g), Borneol 0.001 (mg/g), Cedrol 0.001 (mg/g), D-Limonene 3.07 (mg/g), Caryophyllene oxide 0.039 (mg/g), BetaCaryophyllene 0.68 (mg/g), alpha-Farnesene 0.008 (mg/g), beta-Farnesene 0.008 (mg/g), Humulene 0.106 (mg/g), beta-Ocimene 0.085 (mg/g), alpha-Ocimene 0.009 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Immunosuppressive, Anti-anxiety (Anxiolytic), Appetite Stimulant, Bronchodilator, Anti-spasmotic, Anti-ischemic, Anti-emetic, Neuroprotective

### Formula #72

(a) one, two, three, 4, 5, 6, 7, 8, 9, 10 or all of the following terepenes: Eucalyptol; Linalool; Camphene; alpha-Pinene; gamma-Terpinene; alpha-Terpinene; p-Cymene; Terpinolene; beta-Pinene; Fenchol; Terpineol; 3-Carene; Myrcene; Borneol; D-Limonene; alpha-Cedrene; Caryophyllene oxide; BetaCaryophyllene; Humulene; cis-Nerolidol; cis-beta-ocimene; trans-beta-Ocimene; Valencene; Sabinene Hydrate;

Optionally formulated as: Eucalyptol 0.96 (mg/g), Linalool 8.05 (mg/g), Camphene 6.66 (mg/g), alpha-Pinene 35.09 (mg/g), gamma-Terpinene 0.35 (mg/g), alpha-Terpinene 0.23 (mg/g), p-Cymene 0.11 (mg/g), Terpinolene 3.14 (mg/g), beta-Pinene 74.08 (mg/g), Fenchol 3.98 (mg/g), Terpineol 0.6 (mg/g), 3-Carene 0.06 (mg/g), Myrcene 142.81 (mg/g), Borneol 0.05 (mg/g), D-Limonene 628.16 (mg/g), alpha-Cedrene 0.64 (mg/g), Caryophyllene oxide 0.31 (mg/g), BetaCaryophyllene 20.59 (mg/g), Humulene 3.29 (mg/g), cis-Nerolidol 0.15 (mg/g), cis-beta-ocimene 0.49 (mg/g), trans-beta-Ocimene 2.1 (mg/g), Valencene 0.31 (mg/g), Sabinene Hydrate 0.04 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-spasmotic, Appetite Stimulant, Bronchodilator, Anti-emetic, Anti-ischemic, Anti-psychotic, Anti-anxiety (Anxiolytic), Immunosuppressive

### Formula #73

(a) one, two, three, 4, 5, 6, 7, 8, 9, 10 or all of the following terepenes: Camphor; Linalool; Citronellol; Fenchone; Fenchol; Terpineol; Borneol; Caryophyllene oxide; Bisabolene; BetaCaryophyllene; beta-Farnesene; Humulene; cis-Nerolidol; Valencene; Camphene; alpha-Pinene; beta-Pinene; Myrcene; D-Limonene;

Optionally formulated as: Camphor 0.1 (mg/g), Linalool 13.1 (mg/g), Citronellol 0.6 (mg/g), Fenchone 1.4 (mg/g), Fenchol 7.1 (mg/g), Terpineol 2.6 (mg/g), Borneol 1.2 (mg/g), Caryophyllene oxide 6.1 (mg/g), Bisabolene 5 (mg/g), BetaCaryophyllene 78.4 (mg/g), beta-Farnesene 2.2 (mg/g), Humulene 17.3 (mg/g), cis-Nerolidol 2.7 (mg/g), Valencene 2.1 (mg/g), Camphene 2.1 (mg/g), alpha-Pinene 51.8 (mg/g), beta-Pinene 31.8 (mg/g), Myrcene 426.7 (mg/g), D-Limonene 93 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Anti-anxiety (Anxiolytic), Appetite Stimulant, Anti-psychotic, Anti-spasmotic, Anti-ischemic, Neuroprotective, Anti-emetic, Bronchodilator, Immunosuppressive

### Formula #74

(a) one, two, three, 4, 5, 6, 7, 8, 9, 10 or all of the following terepenes: Terpinolene; Terpineol; Valencene; Camphor; Linalool; Camphene; alpha-Pinene; Citronellol; 4-Carvomenthenol; Fenchone; beta-Pinene; Fenchol; Myrcene; Borneol; D-Menthol; D-Limonene; Nerol; Caryophyllene oxide; Bisabolene; BetaCaryophyllene; alpha-Farnesene; beta-Farnesene; Humulene; beta-Ocimene; Nerolidol; cis-Nerolidol; alpha-Ocimene;

Optionally formulated as: Terpinolene 1.65 (mg/g), Terpineol 5.03 (mg/g), Valencene 3.25 (mg/g), Camphor 0.16 (mg/g), Linalool 16.07 (mg/g), Camphene 2.98 (mg/g), alpha-Pinene 59.22 (mg/g), Citronellol 1.54 (mg/g), 4-Carvomenthenol 0.19 (mg/g), Fenchone 1.72 (mg/g), beta-Pinene 39.69 (mg/g), Fenchol 9.3 (mg/g), Myrcene 409.33 (mg/g), Borneol 1.58 (mg/g), D-Menthol 0.22 (mg/g), D-Limonene 125.77 (mg/g), Nerol 1.54 (mg/g), Caryophyllene oxide 0.25 (mg/g), Bisabolene 18.25 (mg/g), BetaCaryophyllene 43.55 (mg/g), alpha-Farnesene 8.86 (mg/g), beta-Farnesene 7.69 (mg/g), Humulene 10.17 (mg/g), beta-Ocimene 6.48 (mg/g), Nerolidol 0.59 (mg/g), cis-Nerolidol 1.55 (mg/g), alpha-Ocimene 0.19 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Appetite Stimulant, Anti-spasmotic, Anti-emetic, Anti-anxiety (Anxiolytic), Anti-ischemic, Immunosuppressive, Anti-psychotic, Neuroprotective, Bronchodilator

### Formula #75

(a) one, two, three, 4, 5, 6, 7, 8, 9, 10 or all of the following terepenes: Linalool; Camphene; alpha-Pinene; p-Cymene; Terpinolene; Fenchone; beta-Pinene; Fenchol; Terpineol; Myrcene; Borneol; D-Limonene; Caryophyllene oxide; BetaCaryophyllene; beta-Farnesene; Humulene; beta-Ocimene; alpha-Ocimene;

Optionally formulated as: Linalool 0.048 (mg/g), Camphene 0.028 (mg/g), alpha-Pinene 0.974 (mg/g), p-Cymene 0.002 (mg/g), Terpinolene 0.015 (mg/g), Fenchone 0.002 (mg/g), beta-Pinene 0.471 (mg/g), Fenchol 0.022 (mg/g), Terpineol 0.003 (mg/g), Myrcene 6.654 (mg/g), Borneol 0.001 (mg/g), D-Limonene 0.954 (mg/g), Caryophyllene oxide 0.015 (mg/g), BetaCaryophyllene 0.203 (mg/g), beta-Farnesene 0.006 (mg/g), Humulene 0.038 (mg/g), beta-Ocimene 0.435 (mg/g), alpha-Ocimene 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Bronchodilator, Anti-ischemic, Immunosuppressive, Anti-spasmotic, Anti-emetic, Anti-psychotic, Neuroprotective, Appetite Stimulant

### Formula #76

(a) one, two, three, 4, 5, 6, 7, 8, 9, 10 or all of the following terepenes: Linalool; Camphene; alpha-Pinene; gamma-Terpinene; p-Cymene; Terpinolene; Fenchone; beta-Pinene; Fenchol; Terpineol; Myrcene; Borneol; Cedrol; D-Limonene; Geranyl Acetate; Caryophyllene oxide; BetaCaryophyllene; Humulene; beta-Ocimene;

Optionally formulated as: Linalool 0.051 (mg/g), Camphene 0.056 (mg/g), alpha-Pinene 1.381 (mg/g), gamma-Terpinene 0.002 (mg/g), p-Cymene 0.006 (mg/g), Terpinolene 0.022 (mg/g), Fenchone 0.003 (mg/g), beta-Pinene 0.985 (mg/g), Fenchol 0.039 (mg/g), Terpineol 0.005 (mg/g), Myrcene 5.513 (mg/g), Borneol 0.001 (mg/g), Cedrol 0.001 (mg/g), D-Limonene 1.517 (mg/g), Geranyl Acetate 0.001 (mg/g), Caryophyllene oxide 0.022 (mg/g), BetaCaryophyllene 0.142 (mg/g), Humulene 0.031 (mg/g), beta-Ocimene 0.168 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-spasmotic, Appetite Stimulant, Bronchodilator, Anti-emetic, Anti-psychotic, Neuroprotective, Anti-anxiety (Anxiolytic), Anti-ischemic

### Formula #77

(a) one, two, three, 4, 5, 6, 7, 8, 9, 10 or all of the following terepenes: Linalool; alpha-Phellandrene; gamma-Terpinene; p-Cymene; Terpinolene; gamma-Terpineol; Fenchone; Fenchol; Limonene; 3-Carene; Borneol; Cedrol; Guaiol; Caryophyllene oxide; BetaCaryophyllene; alpha-Farnesene; beta-Farnesene; Humulene; beta-Ocimene; trans-beta-Ocimene; Camphene; alpha-Pinene; beta-Pinene; Myrcene;

Optionally formulated as: Linalool 4.1 (mg/g), alpha-Phellandrene 0.8 (mg/g), gamma-Terpinene 0.5 (mg/g), p-Cymene 0.8 (mg/g), Terpinolene 26 (mg/g), gamma-Terpineol 1.2 (mg/g), Fenchone 0.5 (mg/g), Fenchol 4.8 (mg/g), Limonene 270.5 (mg/g), 3-Carene 0.9 (mg/g), Borneol 0.3 (mg/g), Cedrol 0.1 (mg/g), Guaiol 0.1 (mg/g), Caryophyllene oxide 1.5 (mg/g), BetaCaryophyllene 24 (mg/g), alpha-Farnesene 3.2 (mg/g), beta-Farnesene 11.1 (mg/g), Humulene 5.5 (mg/g), beta-Ocimene 42.8 (mg/g), trans-beta-Ocimene 1 (mg/g), Camphene 4.4 (mg/g), alpha-Pinene 55.6 (mg/g), beta-Pinene 63.4 (mg/g), Myrcene 489.2 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-inflammatory, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Bronchodilator, Anti-spasmotic, Anti-psychotic, Appetite Stimulant, Anti-ischemic, Immunosuppressive, Neuroprotective, Anti-emetic

### Formula #78

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Citronellol; Terpinolene; Fenchone; beta-Pinene; Fenchol; Terpineol; Sabinene; 3-Carene; Myrcene; Borneol; Alloaromadendrene; Guaiol; D-Limonene; Caryophyllene oxide; BetaCaryophyllene; alpha-Farnesene; beta-Farnesene; Humulene; beta-Ocimene; alpha-Ocimene;

Optionally formulated as: Linalool 0.082 (mg/g), Camphene 0.01 (mg/g), alpha-Pinene 0.104 (mg/g), alpha-Phellandrene 0.142 (mg/g), gamma-Terpinene 0.036 (mg/g), alpha-Terpinene 0.073 (mg/g), p-Cymene 0.004 (mg/g), Citronellol 0.004 (mg/g), Terpinolene 3.783 (mg/g), Fenchone 0.004 (mg/g), beta-Pinene 0.162 (mg/g), Fenchol 0.031 (mg/g), Terpineol 0.02 (mg/g), Sabinene 0.005 (mg/g), 3-Carene 0.082 (mg/g), Myrcene 1.027 (mg/g), Borneol 0.002 (mg/g), Alloaromadendrene 0.051 (mg/g), Guaiol 0.005 (mg/g), D-Limonene 0.61 (mg/g), Caryophyllene oxide 0.005 (mg/g), BetaCaryophyllene 1.406 (mg/g), alpha-Farnesene 0.119 (mg/g), beta-Farnesene 0.277 (mg/g), Humulene 0.336 (mg/g), beta-Ocimene 0.878 (mg/g), alpha-Ocimene 0.009 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Neuroprotective, Appetite Stimulant, Bronchodilator, Anti-spasmotic, Anti-ischemic, Immunosuppressive, Anti-anxiety (Anxiolytic), Anti-psychotic, Anti-emetic.

### Formula #79

(a) one, two, three, 4, 5, 6, 7, 8, 9, 10 or all of the following terepenes: Linalool; Camphene; alpha-Pinene; gamma-Terpinene; p-Cymene; Terpinolene; Fenchone; beta-Pinene; Fenchol; Terpineol; Myrcene; Borneol; D-Limonene; Caryophyllene oxide; BetaCaryophyllene; alpha-Farnesene; Humulene; beta-Ocimene; alpha-Ocimene;

Optionally formulated as: Linalool 0.16 (mg/g), Camphene 0.071 (mg/g), alpha-Pinene 0.1865 (mg/g), gamma-Terpinene 0.0015 (mg/g), p-Cymene 0.004 (mg/g), Terpinolene 0.034 (mg/g), Fenchone 0.018 (mg/g), beta-Pinene 0.561 (mg/g), Fenchol 0.1335 (mg/g), Terpineol 0.0175 (mg/g), Myrcene 2.367 (mg/g), Borneol 0.003 (mg/g), D-Limonene 6.1175 (mg/g), Caryophyllene oxide 0.0155 (mg/g), BetaCaryophyllene 0.2355 (mg/g), alpha-Farnesene 0.002 (mg/g), Humulene 0.04 (mg/g), beta-Ocimene 0.004 (mg/g), alpha-Ocimene 0.011 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Appetite Stimulant, Anti-ischemic, Anti-anxiety (Anxiolytic), Immunosuppressive, Anti-spasmotic, Anti-psychotic, Anti-emetic, Neuroprotective.

### Formula #80

(a) one, two, three, 4, 5, 6, 7, 8, 9, 10 or all of the following terepenes: Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; D-Limonene; Caryophyllene oxide; BetaCaryophyllene; Humulene; beta-Ocimene;

Optionally formulated as: Linalool 0.095 (mg/g), Camphene 0.043 (mg/g), alpha-Pinene 1.22 (mg/g), alpha-Phellandrene 0.003 (mg/g), gamma-Terpinene 0.002 (mg/g), p-Cymene 0.005 (mg/g), Terpinolene 0.056 (mg/g), Fenchone 0.003 (mg/g), beta-Pinene 0.888 (mg/g), Fenchol 0.031 (mg/g), Terpineol 0.004 (mg/g), 3-Carene 0.004 (mg/g), Myrcene 6.286 (mg/g), Borneol 0.001 (mg/g), D-Limonene 0.981 (mg/g), Caryophyllene oxide 0.009 (mg/g), BetaCaryophyllene 0.14 (mg/g), Humulene 0.025 (mg/g), beta-Ocimene 0.013 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-emetic, Anti-ischemic, Anti-anxiety (Anxiolytic), Anti-psychotic, Neuroprotective, Anti-spasmotic, Bronchodilator, Appetite Stimulant.

### Formula #81

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; D-Limonene; Caryophyllene oxide; BetaCaryophyllene; alpha-Farnesene; beta-Farnesene; Humulene; beta-Ocimene; cis-Nerolidol;

Optionally formulated as: Linalool 0.338 (mg/g), Camphene 0.023 (mg/g), alpha-Pinene 0.708 (mg/g), alpha-Phellandrene 0.016 (mg/g), gamma-Terpinene 0.004 (mg/g), alpha-Terpinene 0.007 (mg/g), p-Cymene 0.006 (mg/g), Terpinolene 0.385 (mg/g), Fenchone 0.018 (mg/g), beta-Pinene 0.405 (mg/g), Fenchol 0.073 (mg/g), Terpineol 0.041 (mg/g), 3-Carene 0.014 (mg/g), Myrcene 5.779 (mg/g), Borneol 0.004 (mg/g), D-Limonene 1.482 (mg/g), Caryophyllene oxide 0.015 (mg/g), BetaCaryophyllene 0.455 (mg/g), alpha-Farnesene 0.03 (mg/g), beta-Farnesene 0.038 (mg/g), Humulene 0.108 (mg/g), beta-Ocimene 0.038 (mg/g), cis-Nerolidol 0.002 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Appetite Stimulant, Anti-psychotic, Anti-anxiety (Anxiolytic), Bronchodilator, Anti-spasmotic, Neuroprotective, Immunosuppressive, Anti-emetic.

### Formula #82

(a) one, two, three, 4, 5, 6, 7, 8, 9, 10 or all of the following terepenes: Linalool; Camphene; alpha-Pinene; alpha-Phellandrene; gamma-Terpinene; p-Cymene; Terpinolene; Fenchone; beta-Pinene; Fenchol; Terpineol; 3-Carene; Myrcene; Borneol; D-Limonene; Caryophyllene oxide; BetaCaryophyllene; alpha-Farnesene; beta-Farnesene; Humulene; beta-Ocimene;

Optionally formulated as: Linalool 0.21 (mg/g), Camphene 0.022 (mg/g), alpha-Pinene 0.689 (mg/g), alpha-Phellandrene 0.004 (mg/g), gamma-Terpinene 0.002 (mg/g), p-Cymene 0.003 (mg/g), Terpinolene 0.142 (mg/g), Fenchone 0.01 (mg/g), beta-Pinene 0.373 (mg/g), Fenchol 0.05 (mg/g), Terpineol 0.031 (mg/g), 3-Carene 0.004 (mg/g), Myrcene 5.73 (mg/g), Borneol 0.003 (mg/g), D-Limonene 1.402 (mg/g), Caryophyllene oxide 0.019 (mg/g), BetaCaryophyllene 0.965 (mg/g), alpha-Farnesene 0.067 (mg/g), beta-Farnesene 0.029 (mg/g), Humulene 0.227 (mg/g), beta-Ocimene 0.019 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-psychotic, Anti-ischemic, Anti-spasmotic, Anti-emetic, Bronchodilator, Anti-anxiety (Anxiolytic), Immunosuppressive, Neuroprotective.

### Formula #83

(a) one, two, three, 4, 5, 6, 7, 8, 9, 10 or all of the following terepenes: Linalool; Camphene; alpha-Pinene; alpha-Phellandrene; gamma-Terpinene; alpha-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; cis-Nerolidol;

Optionally formulated as: Linalool 0.334 (mg/g), Camphene 0.0255 (mg/g), alpha-Pinene 0.7095 (mg/g), alpha-Phellandrene 0.016 (mg/g), gamma-Terpinene 0.004 (mg/g), alpha-Terpinene 0.007 (mg/g), p-Cymene 0.006 (mg/g), Terpinolene 0.38 (mg/g), Fenchone 0.0185 (mg/g), beta-Pinene 0.4075 (mg/g), Fenchol 0.073 (mg/g), Terpineol 0.0405 (mg/g), 3-Carene 0.014 (mg/g), Myrcene 5.7685 (mg/g), Borneol 0.004 (mg/g), Cedrol 0.0005 (mg/g), D-Limonene 1.4795 (mg/g), Caryophyllene oxide 0.0155 (mg/g), BetaCaryophyllene 0.457 (mg/g), alpha-Farnesene 0.031 (mg/g), beta-Farnesene 0.0385 (mg/g), Humulene 0.109 (mg/g), beta-Ocimene 0.0405 (mg/g), cis-Nerolidol 0.001 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-spasmotic, Anti-anxiety (Anxiolytic), Appetite Stimulant, Anti-psychotic, Anti-ischemic, Bronchodilator, Immunosuppressive, Anti-emetic.

### Formula #84

(a) one, two, three, 4, 5, 6, 7, 8, 9, 10 or all of the following terepenes: Eucalyptol; Linalool; Camphene; alpha-Pinene; Pulegone; alpha-Phellandrene; gamma-Terpinene; alpha-Terpinene; p-Cymene; Terpinolene; Fenchone; beta-Pinene; Fenchol; Terpineol; Sabinene; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.023 (mg/g), Linalool 0.058 (mg/g), Camphene 0.036 (mg/g), alpha-Pinene 0.489 (mg/g), Pulegone 0.002 (mg/g), alpha-Phellandrene 0.181 (mg/g), gamma-Terpinene 0.055 (mg/g), alpha-Terpinene 0.089 (mg/g), p-Cymene 0.075 (mg/g), Terpinolene 3.651 (mg/g), Fenchone 0.005 (mg/g), beta-Pinene 0.86 (mg/g), Fenchol 0.022 (mg/g), Terpineol 0.008 (mg/g), Sabinene 0.02 (mg/g), 3-Carene 0.249 (mg/g), Myrcene 2.332 (mg/g), Borneol 0.001 (mg/g), Cedrol 0.001 (mg/g), D-Limonene 0.885 (mg/g), Caryophyllene oxide 0.02 (mg/g), BetaCaryophyllene 0.18 (mg/g), alpha-Farnesene 0.005 (mg/g), beta-Farnesene 0.009 (mg/g), Humulene 0.031 (mg/g), beta-Ocimene 0.691 (mg/g), alpha-Ocimene 0.009 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-spasmotic, Immunosuppressive, Anti-emetic, Anti-anxiety (Anxiolytic), Appetite Stimulant, Anti-psychotic, Anti-ischemic, Neuroprotective.

### Formula #85

(a) one, two, three, 4, 5, 6, 7, 8, 9, 10 or all of the following terepenes: Camphor; Linalool; Camphene; alpha-Pinene; Citronellol; Terpinolene; Fenchone; beta-Pinene; Fenchol; Terpineol; Myrcene; Borneol; D-Menthol; D-Limonene; Caryophyllene oxide; Bisabolene; BetaCaryophyllene; alpha-Farnesene; beta-Farnesene; Humulene; beta-Ocimene; cis-Nerolidol; Valencene;

Optionally formulated as: Camphor 0.2 (mg/g), Linalool 80.6 (mg/g), Camphene 3.7 (mg/g), alpha-Pinene 15.3 (mg/g), Citronellol 1.5 (mg/g), Terpinolene 3.6 (mg/g), Fenchone 2.4 (mg/g), beta-Pinene 22.3 (mg/g), Fenchol 12.9 (mg/g), Terpineol 7.7 (mg/g), Myrcene 62 (mg/g), Borneol 2.1 (mg/g), D-Menthol 0.3 (mg/g), D-Limonene 169.8 (mg/g), Caryophyllene oxide 1.7 (mg/g), Bisabolene 24.9 (mg/g), BetaCaryophyllene 156.2 (mg/g), alpha-Farnesene 7.4 (mg/g), beta-Farnesene 27.1 (mg/g), Humulene 37.5 (mg/g), beta-Ocimene 1.2 (mg/g), cis-Nerolidol 3.1 (mg/g), Valencene 8 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Anti-psychotic, Anti-anxiety (Anxiolytic), Anti-spasmotic, Neuroprotective, Anti-emetic, Appetite Stimulant, Immunosuppressive, Bronchodilator, Anti-ischemic.

### Formula #86

(a) one, two, three, 4, 5, 6, 7, 8, 9, 10 or all of the following terepenes: Eucalyptol; Linalool; Camphene; alpha-Pinene; Terpinolene; Fenchone; beta-Pinene; Fenchol; Terpineol; Myrcene; Borneol; D-Limonene; Caryophyllene oxide; BetaCaryophyllene; alpha-Farnesene; beta-Farnesene; Humulene; beta-Ocimene; Sabinene Hydrate;

Optionally formulated as: Eucalyptol 0.013 (mg/g), Linalool 0.527 (mg/g), Camphene 0.025 (mg/g), alpha-Pinene 0.935 (mg/g), Terpinolene 0.024 (mg/g), Fenchone 0.008 (mg/g), beta-Pinene 0.371 (mg/g), Fenchol 0.054 (mg/g), Terpineol 0.023 (mg/g), Myrcene 5.392 (mg/g), Borneol 0.004 (mg/g), D-Limonene 0.574 (mg/g), Caryophyllene oxide 0.016 (mg/g), BetaCaryophyllene 1.085 (mg/g), alpha-Farnesene 0.089 (mg/g), beta-Farnesene 0.31 (mg/g), Humulene 0.248 (mg/g), beta-Ocimene 0.27 (mg/g), Sabinene Hydrate 0.005 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Bronchodilator, Anti-psychotic, Anti-anxiety (Anxiolytic), Appetite Stimulant, Anti-emetic, Anti-ischemic, Anti-spasmotic, Immunosuppressive.

### Formula #87

(a) one, two, three, 4, 5, 6, 7, 8, 9, 10 or all of the following terepenes: Eucalyptol; Linalool; Camphene; alpha-Pinene; gamma-Terpinene; p-Cymene; Terpinolene; Fenchone; beta-Pinene; Fenchol; Terpineol; 3-Carene; Myrcene; Borneol; Cedrol; D-Limonene; Caryophyllene oxide; BetaCaryophyllene; alpha-Farnesene; beta-Farnesene; Humulene; beta-Ocimene; alpha-Ocimene;

Optionally formulated as: Eucalyptol 0.023 (mg/g), Linalool 0.106 (mg/g), Camphene 0.048 (mg/g), alpha-Pinene 1.106 (mg/g), gamma-Terpinene 0.002 (mg/g), p-Cymene 0.009 (mg/g), Terpinolene 0.022 (mg/g), Fenchone 0.007 (mg/g), beta-Pinene 1.151 (mg/g), Fenchol 0.074 (mg/g), Terpineol 0.015 (mg/g), 3-Carene 0.002 (mg/g), Myrcene 4.356 (mg/g), Borneol 0.002 (mg/g), Cedrol 0.002 (mg/g), D-Limonene 2.311 (mg/g), Caryophyllene oxide 0.055 (mg/g), BetaCaryophyllene 0.547 (mg/g), alpha-Farnesene 0.012 (mg/g), beta-Farnesene 0.006 (mg/g), Humulene 0.097 (mg/g), beta-Ocimene 0.039 (mg/g), alpha-Ocimene 0.006 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-anxiety (Anxiolytic), Appetite Stimulant, Anti-psychotic, Immunosuppressive, Neuroprotective, Anti-ischemic, Anti-spasmotic, Anti-emetic.

### Formula #88

(a) one, two, three, 4, 5, 6, 7, 8, 9, 10 or all of the following terepenes: Linalool; alpha-Pinene; alpha-Phellandrene; gamma-Terpinene; alpha-Terpinene; 4-Carvomenthenol; Terpinolene; beta-Pinene; Terpineol; Sabinene; Myrcene; D-Limonene; Humulene; beta-Ocimene; alpha-Ocimene; Valencene; Camphor; Camphene; p-Cymene; Fenchol; 3-Carene; Borneol; Geraniol; Caryophyllene oxide; Bisabolene; BetaCaryophyllene; alpha-Farnesene; beta-Farnesene;

Optionally formulated as: Linalool 12.75 (mg/g), alpha-Pinene 21.25 (mg/g), alpha-Phellandrene 8.69 (mg/g), gamma-Terpinene 1.04 (mg/g), alpha-Terpinene 2.65 (mg/g), 4-Carvomenthenol 0.43 (mg/g), Terpinolene 152.92 (mg/g), beta-Pinene 34.15 (mg/g), Terpineol 1.68 (mg/g), Sabinene 0.53 (mg/g), Myrcene 132.37 (mg/g), D-Limonene 25.49 (mg/g), Humulene 7.04 (mg/g), beta-Ocimene 28.55 (mg/g), alpha-Ocimene 0.93 (mg/g), Valencene 1.22 (mg/g), Camphor 3.29 (mg/g), Camphene 0.78 (mg/g), p-Cymene 6.1 (mg/g), Fenchol 1.59 (mg/g), 3-Carene 13.59 (mg/g), Borneol 0.24 (mg/g), Geraniol 0.12 (mg/g), Caryophyllene oxide 4.02 (mg/g), Bisabolene 9.61 (mg/g), BetaCaryophyllene 37.16 (mg/g), alpha-Farnesene 2.4 (mg/g), beta-Farnesene 1.93 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-ischemic, Immunosuppressive, Anti-psychotic, Neuroprotective, Anti-emetic, Anti-anxiety (Anxiolytic), Appetite Stimulant, Anti-spasmotic.

### Formula #89

(a) one, two, three, 4, 5, 6, 7, 8, 9, 10 or all of the following terepenes: Linalool; alpha-Pinene; alpha-Phellandrene; gamma-Terpinene; 4-Carvomenthenol; Terpinolene; Fenchone; beta-Pinene; Fenchol; Terpineol; Myrcene; Guaiol; D-Limonene; beta-Farnesene; Humulene; beta-Ocimene; Nerolidol; cis-Nerolidol; alpha-Ocimene; Valencene; Camphor; Eucalyptol; Camphene; 3-Carene; Borneol; Cedrol; alpha-Bisabolol; Caryophyllene oxide; Bisabolene; BetaCaryophyllene; alpha-Farnesene;

Optionally formulated as: Linalool 21.9 (mg/g), alpha-Pinene 8.93 (mg/g), alpha-Phellandrene 0.21 (mg/g), gamma-Terpinene 0.26 (mg/g), 4-Carvomenthenol 0.16 (mg/g), Terpinolene 4.18 (mg/g), Fenchone 1.1 (mg/g), beta-Pinene 17.17 (mg/g), Fenchol 8.03 (mg/g), Terpineol 3.33 (mg/g), Myrcene 283.81 (mg/g), Guaiol 0.51 (mg/g), D-Limonene 114.1 (mg/g), beta-Farnesene 14.43 (mg/g), Humulene 38.67 (mg/g), beta-Ocimene 13.06 (mg/g), Nerolidol 0.7 (mg/g), cis-Nerolidol 1.65 (mg/g), alpha-Ocimene 0.91 (mg/g), Valencene 4.19 (mg/g), Camphor 0.1 (mg/g), Eucalyptol 3.59 (mg/g), Camphene 2.35 (mg/g), 3-Carene 0.12 (mg/g), Borneol 0.9 (mg/g), Cedrol 0.13 (mg/g), alpha-Bisabolol 0.28 (mg/g), Caryophyllene oxide 1.43 (mg/g), Bisabolene 35.26 (mg/g), BetaCaryophyllene 154.01 (mg/g), alpha-Farnesene 14.32 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-emetic, Neuroprotective, Anti-ischemic, Anti-anxiety (Anxiolytic), Appetite Stimulant, Anti-psychotic, Immunosuppressive, Anti-spasmotic.

### Formula #90

(a) one, two, three, 4, 5, 6, 7, 8, 9, 10 or all of the following terepenes: alpha-Pinene; alpha-Phellandrene; gamma-Terpinene; alpha-Terpinene; 4-Carvomenthenol; Terpinolene; beta-Pinene; Terpineol; beta-Ocimene; cis-Nerolidol; alpha-Ocimene; Valencene; Camphor; Linalool; Camphene; Citronellol; Fenchone; Fenchol; 3-Carene; Myrcene; Borneol; D-Menthol; D-Limonene; Geraniol; Nerol; Caryophyllene oxide; Bisabolene; BetaCaryophyllene; alpha-Farnesene; beta-Farnesene; Humulene;

Optionally formulated as: alpha-Pinene 13.62 (mg/g), alpha-Phellandrene 0.17 (mg/g), gamma-Terpinene 0.08 (mg/g), alpha-Terpinene 0.14 (mg/g), 4-Carvomenthenol 0.34 (mg/g), Terpinolene 5.95 (mg/g), beta-Pinene 27.2 (mg/g), Terpineol 9.64 (mg/g), beta-Ocimene 0.94 (mg/g), cis-Nerolidol 2.32 (mg/g), alpha-Ocimene 0.29 (mg/g), Valencene 5.31 (mg/g), Camphor 0.22 (mg/g), Linalool 31.26 (mg/g), Camphene 3.98 (mg/g), Citronellol 1.12 (mg/g), Fenchone 0.76 (mg/g), Fenchol 18.87 (mg/g), 3-Carene 0.15 (mg/g), Myrcene 184.51 (mg/g), Borneol 2.72 (mg/g), D-Menthol 0.38 (mg/g), D-Limonene 228.93 (mg/g), Geraniol 0.67 (mg/g), Nerol 1.1 (mg/g), Caryophyllene oxide 1.11 (mg/g), Bisabolene 28.97 (mg/g), BetaCaryophyllene 98.83 (mg/g), alpha-Farnesene 7.36 (mg/g), beta-Farnesene 11.04 (mg/g), Humulene 23.49 (mg/g),

(b) in alternative embodiment, this formulation is used to treat, ameliorate, lesson the symptoms of, or prevent any one of the following conditions, or be used as an: Anti-inflammatory, Anti-bacterial, Anti-insomnia, Anti-depressant, Anti-convulsive, Anti-cancer, Analgesic, Appetite Suppressant,

(c) in alternative embodiment, this formulation is used to treat, ameliorate, lesson the symptoms of, or prevent any one of the following conditions, or be used as an: Neuroprotective, Anti-anxiety (Anxiolytic), Anti-ischemic, Anti-emetic, Appetite Stimulant, Immunosuppressive, Bronchodilator, Anti-psychotic, Anti-spasmotic.

### Formula #91

(a) one, two, three, 4, 5, 6, 7, 8, 9, 10 or all of the following terepenes: Eucalyptol; Linalool; alpha-Pinene; alpha-Phellandrene; gamma-Terpinene; alpha-Terpinene; 4-Carvomenthenol; Terpinolene; Fenchone; beta-Pinene; Fenchol; Terpineol; Myrcene; D-Menthol; D-Limonene; alpha-Farnesene; beta-Farnesene; Humulene; beta-Ocimene; cis-Nerolidol; alpha-Ocimene; Valencene; Camphor; Camphene; 3-Carene; Borneol; Caryophyllene oxide; Bisabolene; BetaCaryophyllene; Isoborneol, (−)-;

Optionally formulated as: Eucalyptol 0.87 (mg/g), Linalool 85.62 (mg/g), alpha-Pinene 29.19 (mg/g), alpha-Phellandrene 0.19 (mg/g), gamma-Terpinene 0.13 (mg/g), alpha-Terpinene 0.11 (mg/g), 4-Carvomenthenol 0.24 (mg/g), Terpinolene 5.37 (mg/g), Fenchone 4.19 (mg/g), beta-Pinene 48.06 (mg/g), Fenchol 26.58 (mg/g), Terpineol 8.21 (mg/g), Myrcene 65.99 (mg/g), D-Menthol 0.28 (mg/g), D-Limonene 345.24 (mg/g), alpha-Farnesene 12.12 (mg/g), beta-Farnesene 7.55 (mg/g), Humulene 13.37 (mg/g), beta-Ocimene 10.42 (mg/g), cis-Nerolidol 0.68 (mg/g), alpha-Ocimene 0.59 (mg/g), Valencene 2.86 (mg/g), Camphor 0.31 (mg/g), Camphene 7.1 (mg/g), 3-Carene 0.16 (mg/g), Borneol 2.02 (mg/g), Caryophyllene oxide 0.72 (mg/g), Bisabolene 43.31 (mg/g), BetaCaryophyllene 72.67 (mg/g), Isoborneol, (−)-0.05 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-psychotic, Neuroprotective, Appetite Stimulant, Immunosuppressive, Anti-anxiety (Anxiolytic), Anti-emetic, Bronchodilator, Anti-ischemic.

### Formula #92

(a) one, two, three, 4, 5, 6, 7, 8, 9, 10 or all of the following terepenes: alpha-Pinene; alpha-Phellandrene; gamma-Terpinene; alpha-Terpinene; 4-Carvomenthenol; Terpinolene; beta-Pinene; Terpineol; beta-Ocimene; Valencene; Camphor; Linalool; Camphene; Citronellol; Fenchone; Fenchol; 3-Carene; Myrcene; Borneol; D-Menthol; D-Limonene; Nerol; Caryophyllene oxide; Bisabolene; BetaCaryophyllene; alpha-Farnesene; beta-Farnesene; Humulene; cis-Nerolidol; alpha-Ocimene;

Optionally formulated as: alpha-Pinene 31.89 (mg/g), alpha-Phellandrene 0.1 (mg/g), gamma-Terpinene 0.15 (mg/g), alpha-Terpinene 0.07 (mg/g), 4-Carvomenthenol 0.31 (mg/g), Terpinolene 4.15 (mg/g), beta-Pinene 46.57 (mg/g), Terpineol 11.02 (mg/g), beta-Ocimene 11.02 (mg/g), Valencene 2.21 (mg/g), Camphor 0.33 (mg/g), Linalool 105.33 (mg/g), Camphene 6.81 (mg/g), Citronellol 0.81 (mg/g), Fenchone 3.21 (mg/g), Fenchol 29.1 (mg/g), 3-Carene 0.06 (mg/g), Myrcene 84.19 (mg/g), Borneol 2.85 (mg/g), D-Menthol 0.36 (mg/g), D-Limonene 347.05 (mg/g), Nerol 0.79 (mg/g), Caryophyllene oxide 0.37 (mg/g), Bisabolene 97.53 (mg/g), BetaCaryophyllene 48.87 (mg/g), alpha-Farnesene 30.21 (mg/g), beta-Farnesene 9.15 (mg/g), Humulene 11.33 (mg/g), cis-Nerolidol 0.97 (mg/g), alpha-Ocimene 0.63 (mg/g),

(b) in alternative embodiment, this formulation is used to treat, ameliorate, lesson the symptoms of, or prevent any one of the following conditions, or be used as an: Anti-inflammatory, Anti-bacterial, Anti-insomnia, Anti-depressant, Anti-convulsive, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Anti-ischemic, Anti-spasmotic, Neuroprotective, Anti-psychotic, Anti-anxiety (Anxiolytic), Anti-emetic, Appetite Stimulant, Immunosuppressive.

### Formula #93

(a) one, two, three, 4, 5, 6, 7, 8, 9, 10 or all of the following terepenes: Valencene; Terpineol; Terpinolene; gamma-Terpinene; Eucalyptol; Linalool; alpha-Ocimene; alpha-Pinene; alpha-Phellandrene; alpha-Terpinene; p-Cymene; 4-Carvomenthenol; Fenchone; beta-Pinene; Fenchol; 3-Carene; Myrcene; β-Eudesmol; D-Menthol; Guaiol; D-Limonene; Caryophyllene oxide; BetaCaryophyllene; alpha-Farnesene; beta-Farnesene; Humulene; beta-Ocimene; Nerolidol; cis-Nerolidol; Camphene; Borneol; Bisabolene; Camphor;

Optionally formulated as: Valencene 2.38 (mg/g), Terpineol 5.61 (mg/g), Terpinolene 17.32 (mg/g), gamma-Terpinene 0.49 (mg/g), Eucalyptol 0.68 (mg/g), Linalool 26.23 (mg/g), alpha-Ocimene 1.42 (mg/g), alpha-Pinene 31.53 (mg/g), alpha-Phellandrene 1.11 (mg/g), alpha-Terpinene 0.48 (mg/g), p-Cymene 0.27 (mg/g), 4-Carvomenthenol 0.38 (mg/g), Fenchone 0.99 (mg/g), beta-Pinene 21.99 (mg/g), Fenchol 11.81 (mg/g), 3-Carene 0.81 (mg/g), Myrcene 300.23 (mg/g), β-Eudesmol 0.15 (mg/g), D-Menthol 0.42 (mg/g), Guaiol 0.3 (mg/g), D-Limonene 117.35 (mg/g), Caryophyllene oxide 0.67 (mg/g), BetaCaryophyllene 52.47 (mg/g), alpha-Farnesene 3.37 (mg/g), beta-Farnesene 3.02 (mg/g), Humulene 14.01 (mg/g), beta-Ocimene 162.97 (mg/g), Nerolidol 0.56 (mg/g), cis-Nerolidol 1.62 (mg/g), Camphene 2.35 (mg/g), Borneol 2.25 (mg/g), Bisabolene 24 (mg/g), Camphor 0.22 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Immunosuppressive, Appetite Stimulant, Anti-psychotic, Anti-ischemic, Neuroprotective, Anti-emetic, Anti-spasmotic, Bronchodilator.

### Formula #94

(a) one, two, three, 4, 5, 6, 7, 8, 9, 10 or all of the following terepenes: gamma-Terpinene; 4-Carvomenthenol; Terpinolene; beta-Pinene; Terpineol; Valencene; Camphor; Eucalyptol; Linalool; Camphene; alpha-Pinene; cineole; Fenchone; Fenchol; Myrcene; Borneol; β-Eudesmol; Guaiol; D-Limonene; alpha-Bisabolol; Caryophyllene oxide; Bisabolene; BetaCaryophyllene; alpha-Farnesene; beta-Farnesene; Humulene; beta-Ocimene; Nerolidol; cis-Nerolidol; alpha-Ocimene;

Optionally formulated as: gamma-Terpinene 0.2 (mg/g), 4-Carvomenthenol 0.08 (mg/g), Terpinolene 0.84 (mg/g), beta-Pinene 18.08 (mg/g), Terpineol 1.67 (mg/g), Valencene 5.8 (mg/g), Camphor 0.07 (mg/g), Eucalyptol 2.67 (mg/g), Linalool 11.81 (mg/g), Camphene 2.45 (mg/g), alpha-Pinene 18.04 (mg/g), cineole 1.1 (mg/g), Fenchone 0.55 (mg/g), Fenchol 6.01 (mg/g), Myrcene 59.95 (mg/g), Borneol 0.68 (mg/g), β-Eudesmol 0.26 (mg/g), Guaiol 0.5 (mg/g), D-Limonene 127.48 (mg/g), alpha-Bisabolol 0.2 (mg/g), Caryophyllene oxide 1.98 (mg/g), Bisabolene 46.24 (mg/g), BetaCaryophyllene 281.71 (mg/g), alpha-Farnesene 20.3 (mg/g), beta-Farnesene 27.03 (mg/g), Humulene 83.79 (mg/g), beta-Ocimene 1.91 (mg/g), Nerolidol 1.09 (mg/g), cis-Nerolidol 2.81 (mg/g), alpha-Ocimene 0.27 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Bronchodilator, Anti-spasmotic, Anti-emetic, Neuroprotective, Anti-psychotic, Immunosuppressive, Appetite Stimulant, Anti-anxiety (Anxiolytic).

### Formula #95

(a) one, two, three, 4, 5, 6, 7, 8, 9, 10 or all of the following terepenes: Eucalyptol; Linalool; Camphene; alpha-Pinene; Terpinolene; Fenchone; beta-Pinene; Fenchol; Terpineol; Myrcene; Borneol; D-Limonene; Caryophyllene oxide; Bisabolene; BetaCaryophyllene; beta-Farnesene; Humulene; beta-Ocimene; Nerolidol; cis-Nerolidol;

Optionally formulated as: Eucalyptol 1.24 (mg/g), Linalool 2.22 (mg/g), Camphene 1.87 (mg/g), alpha-Pinene 116.53 (mg/g), Terpinolene 0.72 (mg/g), Fenchone 0.24 (mg/g), beta-Pinene 55.89 (mg/g), Fenchol 1.79 (mg/g), Terpineol 0.06 (mg/g), Myrcene 493.08 (mg/g), Borneol 0.22 (mg/g), D-Limonene 37.52 (mg/g), Caryophyllene oxide 0.38 (mg/g), Bisabolene 11.32 (mg/g), BetaCaryophyllene 72.84 (mg/g), beta-Farnesene 1.78 (mg/g), Humulene 23.56 (mg/g), beta-Ocimene 0.64 (mg/g), Nerolidol 0.27 (mg/g), cis-Nerolidol 0.97 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Neuroprotective, Appetite Stimulant, Anti-anxiety (Anxiolytic), Bronchodilator, Anti-ischemic, Anti-spasmotic, Anti-emetic, Immunosuppressive.

### Formula #96

(a) one, two, three, 4, 5, 6, 7, 8, 9, 10 or all of the following terepenes: gamma-Terpinene; Terpinolene; Terpineol; Valencene; Camphor; Eucalyptol; Linalool; Camphene; alpha-Pinene; alpha-Phellandrene; alpha-Terpinene; Citronellol; cineole; 4-Carvomenthenol; Fenchone; beta-Pinene; Fenchol; 3-Carene; Myrcene; D-Limonene; Caryophyllene oxide; BetaCaryophyllene; alpha-Farnesene; beta-Farnesene; Humulene; beta-Ocimene; cis-Nerolidol; alpha-Ocimene; Borneol; Bisabolene;

Optionally formulated as: gamma-Terpinene 0.21 (mg/g), Terpinolene 6.52 (mg/g), Terpineol 4.96 (mg/g), Valencene 2.02 (mg/g), Camphor 0.19 (mg/g), Eucalyptol 1.06 (mg/g), Linalool 57.82 (mg/g), Camphene 8.9 (mg/g), alpha-Pinene 32.08 (mg/g), alpha-Phellandrene 0.16 (mg/g), alpha-Terpinene 0.19 (mg/g), Citronellol 0.29 (mg/g), cineole 6.08 (mg/g), 4-Carvomenthenol 0.23 (mg/g), Fenchone 2.56 (mg/g), beta-Pinene 57.04 (mg/g), Fenchol 18.07 (mg/g), 3-Carene 0.17 (mg/g), Myrcene 93.45 (mg/g), D-Limonene 355.77 (mg/g), Caryophyllene oxide 0.53 (mg/g), BetaCaryophyllene 69.89 (mg/g), alpha-Farnesene 4.49 (mg/g), beta-Farnesene 9.19 (mg/g), Humulene 15.03 (mg/g), beta-Ocimene 0.71 (mg/g), cis-Nerolidol 0.86 (mg/g), alpha-Ocimene 0.55 (mg/g), Borneol 1.72 (mg/g), Bisabolene 10.6 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Immunosuppressive, Appetite Stimulant, Anti-psychotic, Anti-anxiety (Anxiolytic), Neuroprotective, Anti-emetic, Anti-spasmotic, Anti-ischemic.

### Formula #97

(a) one, two, three, 4, 5, 6, 7, 8, 9, 10 or all of the following terepenes: gamma-Terpinene; 4-Carvomenthenol; Terpinolene; beta-Pinene; Terpineol; Valencene; Camphor; Linalool; Camphene; alpha-Pinene; Fenchone; Fenchol; Myrcene; Borneol; Guaiol; D-Limonene; Caryophyllene oxide; Bisabolene; BetaCaryophyllene; alpha-Farnesene; beta-Farnesene; Humulene; beta-Ocimene; cis-Nerolidol; alpha-Ocimene;

Optionally formulated as: gamma-Terpinene 0.09 (mg/g), 4-Carvomenthenol 0.11 (mg/g), Terpinolene 1.77 (mg/g), beta-Pinene 28.9 (mg/g), Terpineol 4.49 (mg/g), Valencene 2.16 (mg/g), Camphor 0.13 (mg/g), Linalool 49.24 (mg/g), Camphene 4.08 (mg/g), alpha-Pinene 16.96 (mg/g), Fenchone 1.19 (mg/g), Fenchol 9.18 (mg/g), Myrcene 376.79 (mg/g), Borneol 1.12 (mg/g), Guaiol 0.84 (mg/g), D-Limonene 192.51 (mg/g), Caryophyllene oxide 0.52 (mg/g), Bisabolene 28.9 (mg/g), BetaCaryophyllene 49.76 (mg/g), alpha-Farnesene 16.35 (mg/g), beta-Farnesene 3.77 (mg/g), Humulene 15.02 (mg/g), beta-Ocimene 1.4 (mg/g), cis-Nerolidol 0.44 (mg/g), alpha-Ocimene 0.2 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-ischemic, Anti-emetic, Appetite Stimulant, Bronchodilator, Anti-spasmotic, Immunosuppressive, Anti-psychotic, Anti-anxiety (Anxiolytic).

### Formula #98

(a) one, two, three, 4, 5, 6, 7, 8, 9, 10 or all of the following terepenes: Terpinolene; Terpineol; Valencene; Camphor; Linalool; Camphene; alpha-Pinene; Citronellol; 4-Carvomenthenol; Fenchone; beta-Pinene; Fenchol; Myrcene; Borneol; D-Menthol; D-Limonene; Caryophyllene oxide; Bisabolene; BetaCaryophyllene; alpha-Farnesene; beta-Farnesene; Humulene; Nerolidol; cis-Nerolidol; alpha-Ocimene;

Optionally formulated as: Terpinolene 2.07 (mg/g), Terpineol 11.56 (mg/g), Valencene 7.73 (mg/g), Camphor 0.36 (mg/g), Linalool 106.6 (mg/g), Camphene 4.88 (mg/g), alpha-Pinene 15.75 (mg/g), Citronellol 0.66 (mg/g), 4-Carvomenthenol 0.25 (mg/g), Fenchone 2.18 (mg/g), beta-Pinene 28.47 (mg/g), Fenchol 21.08 (mg/g), Myrcene 10.96 (mg/g), Borneol 3.12 (mg/g), D-Menthol 0.27 (mg/g), D-Limonene 202.36 (mg/g), Caryophyllene oxide 0.82 (mg/g), Bisabolene 16.54 (mg/g), BetaCaryophyllene 165.52 (mg/g), alpha-Farnesene 13.67 (mg/g), beta-Farnesene 12.79 (mg/g), Humulene 51.55 (mg/g), Nerolidol 0.93 (mg/g), cis-Nerolidol 0.73 (mg/g), alpha-Ocimene 0.24 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Anti-spasmotic, Anti-psychotic, Anti-emetic, Anti-anxiety (Anxiolytic), Neuroprotective, Anti-ischemic, Appetite Stimulant, Immunosuppressive.

### Formula #99

(a) one, two, three, 4, 5, 6, 7, 8, 9, 10 or all of the following terepenes: 4-Carvomenthenol; Terpinolene; Terpineol; Valencene; Camphor; Linalool; Camphene; alpha-Pinene; Citronellol; Fenchone; beta-Pinene; Fenchol; Myrcene; Borneol; D-Menthol; D-Limonene; Geraniol; Caryophyllene oxide; Bisabolene; BetaCaryophyllene; alpha-Farnesene; beta-Farnesene; Humulene; beta-Ocimene; cis-Nerolidol; alpha-Ocimene; Isoborneol, (−)-;

Optionally formulated as: 4-Carvomenthenol 0.22 (mg/g), Terpinolene 2.64 (mg/g), Terpineol 10.06 (mg/g), Valencene 8.27 (mg/g), Camphor 0.27 (mg/g), Linalool 80.89 (mg/g), Camphene 5.35 (mg/g), alpha-Pinene 19.09 (mg/g), Citronellol 0.46 (mg/g), Fenchone 1.58 (mg/g), beta-Pinene 32.07 (mg/g), Fenchol 18.57 (mg/g), Myrcene 84.27 (mg/g), Borneol 2.66 (mg/g), D-Menthol 0.23 (mg/g), D-Limonene 261.58 (mg/g), Geraniol 0.29 (mg/g), Caryophyllene oxide 2.11 (mg/g), Bisabolene 140.45 (mg/g), BetaCaryophyllene 81.68 (mg/g), alpha-Farnesene 65.13 (mg/g), beta-Farnesene 21.89 (mg/g), Humulene 22 (mg/g), beta-Ocimene 0.29 (mg/g), cis-Nerolidol 0.76 (mg/g), alpha-Ocimene 0.26 (mg/g), Isoborneol, (−)-0.05 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Anti-psychotic, Appetite Stimulant, Immunosuppressive, Anti-spasmotic, Bronchodilator, Anti-emetic, Neuroprotective, Anti-anxiety (Anxiolytic), Anti-ischemic.

### Formula #100

(a) one, two, three, 4, 5, 6, 7, 8, 9, 10 or all of the following terepenes: Sabinene Hydrate; 4-Carvomenthenol; Terpinolene; Terpineol; alpha-Pinene; alpha-Phellandrene; gamma-Terpinene; alpha-Terpinene; beta-Pinene; Sabinene; beta-Ocimene; alpha-Ocimene; Valencene; p-Cymene; cineole; Fenchone; Fenchol; 3-Carene; Camphor; Borneol; β-Eudesmol; D-Menthol; Guaiol; D-Limonene; Caryophyllene oxide; Bisabolene; BetaCaryophyllene; alpha-Farnesene; beta-Farnesene; Humulene; Eucalyptol; Nerolidol; cis-Nerolidol; Linalool; Camphene; Myrcene;

Optionally formulated as: Sabinene Hydrate 0.41 (mg/g), 4-Carvomenthenol 0.62 (mg/g), Terpinolene 139.55 (mg/g), Terpineol 4.15 (mg/g), alpha-Pinene 9.97 (mg/g), alpha-Phellandrene 8.07 (mg/g), gamma-Terpinene 2.09 (mg/g), alpha-Terpinene 3.53 (mg/g), beta-Pinene 15.85 (mg/g), Sabinene 0.44 (mg/g), beta-Ocimene 22.84 (mg/g), alpha-Ocimene 0.31 (mg/g), Valencene 5.4 (mg/g), p-Cymene 1.35 (mg/g), cineole 0.67 (mg/g), Fenchone 0.51 (mg/g), Fenchol 3.96 (mg/g), 3-Carene 6.61 (mg/g), Camphor 0.51 (mg/g), Borneol 0.73 (mg/g), β-Eudesmol 0.37 (mg/g), D-Menthol 0.7 (mg/g), Guaiol 0.62 (mg/g), D-Limonene 37.97 (mg/g), Caryophyllene oxide 3.1 (mg/g), Bisabolene 39.96 (mg/g), BetaCaryophyllene 183.05 (mg/g), alpha-Farnesene 35.36 (mg/g), beta-Farnesene 26.42 (mg/g), Humulene 68.46 (mg/g), Eucalyptol 1.17 (mg/g), Nerolidol 1.61 (mg/g), cis-Nerolidol 4.73 (mg/g), Linalool 5.69 (mg/g), Camphene 0.86 (mg/g), Myrcene 38.69 (mg/g),

(b) in alternative embodiment, this formulation is used to treat, ameliorate, lesson the symptoms of, or prevent any one of the following conditions, or be used as an: Anti-bacterial, Anti-insomnia, Anti-inflammatory, Anti-depressant, Analgesic, Anti-cancer, Appetite Suppressant, Anti-convulsive,

(c) in alternative embodiment, this formulation is used to treat, ameliorate, lesson the symptoms of, or prevent any one of the following conditions, or be used as an: Neuroprotective, Anti-ischemic, Anti-spasmotic, Anti-psychotic, Immunosuppressive, Appetite Stimulant, Anti-emetic, Anti-anxiety (Anxiolytic), Bronchodilator.

### Formula #101

(a) one, two, three, 4, 5, 6, 7, 8, 9, 10 or all of the following terepenes: alpha-Pinene; 4-Carvomenthenol; Terpinolene; beta-Pinene; Terpineol; beta-Ocimene; Valencene; Camphor; Linalool; Camphene; Citronellol; Fenchone; Fenchol; Myrcene; Borneol; D-Menthol; D-Limonene; Caryophyllene oxide; Bisabolene; BetaCaryophyllene; alpha-Farnesene; beta-Farnesene; Humulene; Nerolidol; cis-Nerolidol;

Optionally formulated as: alpha-Pinene 12.76 (mg/g), 4-Carvomenthenol 0.2 (mg/g), Terpinolene 1.82 (mg/g), beta-Pinene 22.5 (mg/g), Terpineol 6.48 (mg/g), beta-Ocimene 0.86 (mg/g), Valencene 11.23 (mg/g), Camphor 0.19 (mg/g), Linalool 86.38 (mg/g), Camphene 3.28 (mg/g), Citronellol 0.2 (mg/g), Fenchone 2.83 (mg/g), Fenchol 11.5 (mg/g), Myrcene 127.43 (mg/g), Borneol 1.82 (mg/g), D-Menthol 0.21 (mg/g), D-Limonene 141.83 (mg/g), Caryophyllene oxide 2.98 (mg/g), Bisabolene 253.42 (mg/g), BetaCaryophyllene 131.83 (mg/g), alpha-Farnesene 60.23 (mg/g), beta-Farnesene 29.2 (mg/g), Humulene 36.19 (mg/g), Nerolidol 6.18 (mg/g), cis-Nerolidol 0.59 (mg/g),

(b) in alternative embodiment, this formulation is used to treat, ameliorate, lesson the symptoms of, or prevent any one of the following conditions, or be used as an: Anti-inflammatory, Anti-bacterial, Anti-insomnia, Anti-depressant, Anti-convulsive, Anti-cancer, Analgesic, Appetite Suppressant,

(c) in alternative embodiment, this formulation is used to treat, ameliorate, lesson the symptoms of, or prevent any one of the following conditions, or be used as an: Anti-spasmotic, Anti-ischemic, Neuroprotective, Anti-anxiety (Anxiolytic), Anti-emetic, Bronchodilator, Appetite Stimulant, Anti-psychotic, Immunosuppressive.

### Formula #102

(a) one, two, three, 4, 5, 6, 7, 8, 9, 10 or all of the following terepenes: Eucalyptol; Linalool; Camphene; alpha-Pinene; alpha-Phellandrene; gamma-Terpinene; alpha-Terpinene; 4-Carvomenthenol; Terpinolene; Fenchone; beta-Pinene; Fenchol; Terpineol; 3-Carene; Myrcene; Borneol; D-Menthol; D-Limonene; Caryophyllene oxide; Bisabolene; BetaCaryophyllene; alpha-Farnesene; beta-Farnesene; Humulene; beta-Ocimene; cis-Nerolidol; Valencene;

Optionally formulated as: Eucalyptol 2.88 (mg/g), Linalool 6.41 (mg/g), Camphene 1.91 (mg/g), alpha-Pinene 7.02 (mg/g), alpha-Phellandrene 0.23 (mg/g), gamma-Terpinene 0.22 (mg/g), alpha-Terpinene 0.13 (mg/g), 4-Carvomenthenol 0.21 (mg/g), Terpinolene 10.75 (mg/g), Fenchone 0.53 (mg/g), beta-Pinene 15.09 (mg/g), Fenchol 5.91 (mg/g), Terpineol 1.41 (mg/g), 3-Carene 0.19 (mg/g), Myrcene 364.95 (mg/g), Borneol 0.77 (mg/g), D-Menthol 0.23 (mg/g), D-Limonene 76.27 (mg/g), Caryophyllene oxide 0.83 (mg/g), Bisabolene 30.58 (mg/g), BetaCaryophyllene 109.24 (mg/g), alpha-Farnesene 8.52 (mg/g), beta-Farnesene 8.8 (mg/g), Humulene 29.17 (mg/g), beta-Ocimene 1.78 (mg/g), cis-Nerolidol 1.1 (mg/g), Valencene 3.3 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-anxiety (Anxiolytic), Immunosuppressive, Anti-ischemic, Appetite Stimulant, Neuroprotective, Bronchodilator, Anti-psychotic, Anti-spasmotic.

### Formula #103

(a) one, two, three, 4, 5, 6, 7, 8, 9, 10 or all of the following terepenes: Caryophyllene oxide; Bisabolene; Nerolidol; cis-Nerolidol; Camphor; Linalool; Camphene; alpha-Pinene; Citronellol; cineole; 4-Carvomenthenol; Terpinolene; Fenchone; beta-Pinene; Fenchol; Terpineol; Myrcene; Borneol; Alloaromadendrene; D-Limonene; BetaCaryophyllene; alpha-Farnesene; beta-Farnesene; Humulene; beta-Ocimene; alpha-Ocimene; Valencene;

Optionally formulated as: Caryophyllene oxide 1.7 (mg/g), Bisabolene 15.6 (mg/g), Nerolidol 0.4 (mg/g), cis-Nerolidol 2.2 (mg/g), Camphor 0.2 (mg/g), Linalool 157.7 (mg/g), Camphene 5 (mg/g), alpha-Pinene 27.5 (mg/g), Citronellol 0.4 (mg/g), cineole 0.9 (mg/g), 4-Carvomenthenol 0.1 (mg/g), Terpinolene 2.1 (mg/g), Fenchone 1.4 (mg/g), beta-Pinene 31.7 (mg/g), Fenchol 13.6 (mg/g), Terpineol 4.5 (mg/g), Myrcene 147.8 (mg/g), Borneol 2.2 (mg/g), Alloaromadendrene 0.4 (mg/g), D-Limonene 164 (mg/g), BetaCaryophyllene 177.7 (mg/g), alpha-Farnesene 1.2 (mg/g), beta-Farnesene 7.3 (mg/g), Humulene 46 (mg/g), beta-Ocimene 6.8 (mg/g), alpha-Ocimene 0.4 (mg/g), Valencene 6.3 (mg/g),

(b) in alternative embodiment, this formulation is used to treat, ameliorate, lesson the symptoms of, or prevent any one of the following conditions, or be used as an: Anti-insomnia, Anti-bacterial, Anti-depressant, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Immunosuppressive, Anti-anxiety (Anxiolytic), Appetite Stimulant, Anti-emetic, Bronchodilator, Anti-psychotic, Anti-spasmotic, Neuroprotective, Anti-ischemic.

### Formula #104

(a) one, two, three, 4, 5, 6, 7, 8, 9, 10 or all of the following terepenes: Caryophyllene oxide; Bisabolene; cis-Nerolidol; Linalool; Camphene; alpha-Pinene; Citronellol; Terpinolene; Fenchone; beta-Pinene; Fenchol; Terpineol; Myrcene; Borneol; D-Limonene; BetaCaryophyllene; alpha-Farnesene; beta-Farnesene; Humulene; beta-Ocimene; Valencene;

Optionally formulated as: Caryophyllene oxide 1.2 (mg/g), Bisabolene 11.6 (mg/g), cis-Nerolidol 0.9 (mg/g), Linalool 18.8 (mg/g), Camphene 3 (mg/g), alpha-Pinene 72.1 (mg/g), Citronellol 1.4 (mg/g), Terpinolene 1.5 (mg/g), Fenchone 2.8 (mg/g), beta-Pinene 42.3 (mg/g), Fenchol 7 (mg/g), Terpineol 4.5 (mg/g), Myrcene 426.4 (mg/g), Borneol 1.4 (mg/g), D-Limonene 96.4 (mg/g), BetaCaryophyllene 93.6 (mg/g), alpha-Farnesene 4.2 (mg/g), beta-Farnesene 7.7 (mg/g), Humulene 22.9 (mg/g), beta-Ocimene 16.6 (mg/g), Valencene 4.2 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Anti-emetic, Anti-ischemic, Neuroprotective, Anti-anxiety (Anxiolytic), Anti-spasmotic, Immunosuppressive, Anti-psychotic, Appetite. Stimulant

### Formula #105

(a) one, two, three, 4, 5, 6, 7, 8, 9, 10 or all of the following terepenes: Caryophyllene oxide; Bisabolene; BetaCaryophyllene; alpha-Farnesene; beta-Farnesene; Humulene; cis-Nerolidol; Valencene; Camphor; Linalool; Camphene; alpha-Pinene; alpha-Phellandrene; gamma-Terpinene; alpha-Terpinene; p-Cymene; 4-Carvomenthenol; Terpinolene; beta-Pinene; Fenchol; Terpineol; Sabinene; 3-Carene; Myrcene; D-Menthol; D-Limonene; beta-Ocimene;

Optionally formulated as: Caryophyllene oxide 5.2 (mg/g), Bisabolene 11.4 (mg/g), BetaCaryophyllene 71.8 (mg/g), alpha-Farnesene 4.1 (mg/g), beta-Farnesene 6 (mg/g), Humulene 19.8 (mg/g), cis-Nerolidol 1.1 (mg/g), Valencene 1.9 (mg/g), Camphor 4.5 (mg/g), Linalool 2.2 (mg/g), Camphene 1.4 (mg/g), alpha-Pinene 80 (mg/g), alpha-Phellandrene 13.1 (mg/g), gamma-Terpinene 1.9 (mg/g), alpha-Terpinene 4.8 (mg/g), p-Cymene 5 (mg/g), 4-Carvomenthenol 0.7 (mg/g), Terpinolene 285.6 (mg/g), beta-Pinene 54.5 (mg/g), Fenchol 3.4 (mg/g), Terpineol 2.9 (mg/g), Sabinene 1.7 (mg/g), 3-Carene 12.5 (mg/g), Myrcene 60.9 (mg/g), D-Menthol 0.8 (mg/g), D-Limonene 43.2 (mg/g), beta-Ocimene 14.7 (mg/g),

(b) in alternative embodiment, this formulation is used to treat, ameliorate, lesson the symptoms of, or prevent any one of the following conditions, or be used as an: Anti-inflammatory, Anti-bacterial, Anti-cancer, Appetite Suppressant, Anti-depressant, Anti-insomnia, Anti-convulsive, Analgesic,

(c) in alternative embodiment, this formulation is used to treat, ameliorate, lesson the symptoms of, or prevent any one of the following conditions, or be used as an: Anti-spasmotic, Anti-psychotic, Immunosuppressive, Bronchodilator, Anti-anxiety (Anxiolytic), Appetite Stimulant, Anti-emetic, Neuroprotective, Anti-ischemic.

### Formula #106

(a) one, two, three, 4, 5, 6, 7, 8, 9, 10 or all of the following terepenes: Camphor; Linalool; Camphene; Terpinolene; Fenchone; beta-Pinene; Fenchol; Terpineol; Myrcene; Borneol; Alloaromadendrene; D-Limonene; Caryophyllene oxide; Bisabolene; BetaCaryophyllene; alpha-Farnesene; beta-Farnesene; Humulene; Nerolidol; cis-Nerolidol; Valencene; alpha-Pinene;

Optionally formulated as: Camphor 0.3 (mg/g), Linalool 43.4 (mg/g), Camphene 4.6 (mg/g), Terpinolene 1 (mg/g), Fenchone 2.3 (mg/g), beta-Pinene 28.9 (mg/g), Fenchol 17.8 (mg/g), Terpineol 6.4 (mg/g), Myrcene 41.2 (mg/g), Borneol 2 (mg/g), Alloaromadendrene 0.6 (mg/g), D-Limonene 183.1 (mg/g), Caryophyllene oxide 20.5 (mg/g), Bisabolene 13.5 (mg/g), BetaCaryophyllene 176.2 (mg/g), alpha-Farnesene 3.5 (mg/g), beta-Farnesene 21.1 (mg/g), Humulene 59.8 (mg/g), Nerolidol 0.3 (mg/g), cis-Nerolidol 0.6 (mg/g), Valencene 3.2 (mg/g), alpha-Pinene 18.9 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-inflammatory, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-anxiety (Anxiolytic), Neuroprotective, Anti-emetic, Immunosuppressive, Anti-spasmotic, Bronchodilator, Anti-psychotic, Appetite Stimulant.

### Formula #107

(a) one, two, three, 4, 5, 6, 7, 8, 9, 10 or all of the following terepenes: Camphor; Linalool; Camphene; Citronellol; 4-Carvomenthenol; Terpinolene; Fenchone; beta-Pinene; Fenchol; Terpineol; Myrcene; Borneol; D-Menthol; D-Limonene; Geraniol; Caryophyllene oxide; Bisabolene; BetaCaryophyllene; alpha-Farnesene; beta-Farnesene; Humulene; beta-Ocimene; cis-Nerolidol; alpha-Ocimene; Valencene; alpha-Pinene;

Optionally formulated as: Camphor 0.5 (mg/g), Linalool 88.3 (mg/g), Camphene 7.2 (mg/g), Citronellol 0.2 (mg/g), 4-Carvomenthenol 0.3 (mg/g), Terpinolene 4.3 (mg/g), Fenchone 5.2 (mg/g), beta-Pinene 46.3 (mg/g), Fenchol 26.3 (mg/g), Terpineol 10.6 (mg/g), Myrcene 65 (mg/g), Borneol 3.1 (mg/g), D-Menthol 0.4 (mg/g), D-Limonene 254.6 (mg/g), Geraniol 0.1 (mg/g), Caryophyllene oxide 4.4 (mg/g), Bisabolene 11.7 (mg/g), BetaCaryophyllene 88.4 (mg/g), alpha-Farnesene 3.1 (mg/g), beta-Farnesene 14.2 (mg/g), Humulene 20.6 (mg/g), beta-Ocimene 7.9 (mg/g), cis-Nerolidol 0.6 (mg/g), alpha-Ocimene 0.4 (mg/g), Valencene 4.5 (mg/g), alpha-Pinene 33.2 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Bronchodilator, Anti-spasmotic, Anti-emetic, Immunosuppressive, Neuroprotective, Appetite Stimulant, Anti-psychotic, Anti-anxiety (Anxiolytic), Anti-ischemic.

### Formula #108

(a) one, two, three, 4, 5, 6, 7, 8, 9, 10 or all of the following terepenes: 4-Carvomenthenol; Terpineol; D-Menthol; Caryophyllene oxide; Bisabolene; BetaCaryophyllene; alpha-Farnesene; beta-Farnesene; Humulene; cis-Nerolidol; Valencene; Camphor; Linalool; Camphene; alpha-Pinene; alpha-Phellandrene; gamma-Terpinene; Terpinolene; Fenchone; beta-Pinene; Fenchol; 3-Carene; Myrcene; Borneol; D-Limonene; beta-Ocimene;

Optionally formulated as: 4-Carvomenthenol 0.2 (mg/g), Terpineol 4.8 (mg/g), D-Menthol 0.3 (mg/g), Caryophyllene oxide 1.6 (mg/g), Bisabolene 27.8 (mg/g), BetaCaryophyllene 162.3 (mg/g), alpha-Farnesene 14.6 (mg/g), beta-Farnesene 7.5 (mg/g), Humulene 36.6 (mg/g), cis-Nerolidol 2 (mg/g), Valencene 4.5 (mg/g), Camphor 0.2 (mg/g), Linalool 43.8 (mg/g), Camphene 3.9 (mg/g), alpha-Pinene 21.3 (mg/g), alpha-Phellandrene 0.3 (mg/g), gamma-Terpinene 0.3 (mg/g), Terpinolene 9.2 (mg/g), Fenchone 1.9 (mg/g), beta-Pinene 24.9 (mg/g), Fenchol 10.7 (mg/g), 3-Carene 0.2 (mg/g), Myrcene 211.7 (mg/g), Borneol 1.8 (mg/g), D-Limonene 204.4 (mg/g), beta-Ocimene 6.2 (mg/g),

(b) in alternative embodiment, this formulation is used to treat, ameliorate, lesson the symptoms of, or prevent any one of the following conditions, or be used as an: Anti-inflammatory, Anti-bacterial, Anti-cancer, Appetite Suppressant, Anti-depressant, Anti-insomnia, Anti-convulsive, Analgesic,

(c) in alternative embodiment, this formulation is used to treat, ameliorate, lesson the symptoms of, or prevent any one of the following conditions, or be used as an: Anti-ischemic, Anti-spasmotic, Appetite Stimulant, Immunosuppressive, Anti-emetic, Anti-psychotic, Bronchodilator, Neuroprotective, Anti-anxiety (Anxiolytic).

### Formula #109

(a) one, two, three, 4, 5, 6, 7, 8, 9, 10 or all of the following terepenes: alpha-Bisabolol; Camphor; Linalool; Camphene; alpha-Pinene; Terpinolene; Fenchone; beta-Pinene; Fenchol; Terpineol; Myrcene; Borneol; D-Limonene; Caryophyllene oxide; Bisabolene; BetaCaryophyllene; alpha-Farnesene; beta-Farnesene; Humulene; beta-Ocimene; Nerolidol;

Optionally formulated as: alpha-Bisabolol 0.44 (mg/g), Camphor 0.01 (mg/g), Linalool 1.46 (mg/g), Camphene 0.09 (mg/g), alpha-Pinene 0.6 (mg/g), Terpinolene 0.14 (mg/g), Fenchone 0.04 (mg/g), beta-Pinene 0.61 (mg/g), Fenchol 0.55 (mg/g), Terpineol 0.42 (mg/g), Myrcene 0.72 (mg/g), Borneol 0.1 (mg/g), D-Limonene 2.38 (mg/g), Caryophyllene oxide 0.13 (mg/g), Bisabolene 0.65 (mg/g), BetaCaryophyllene 4.21 (mg/g), alpha-Farnesene 0.45 (mg/g), beta-Farnesene 0.55 (mg/g), Humulene 1.13 (mg/g), beta-Ocimene 0.08 (mg/g), Nerolidol 0.11 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-psychotic, Immunosuppressive, Anti-anxiety (Anxiolytic), Appetite Stimulant, Bronchodilator, Anti-emetic, Neuroprotective, Anti-spasmotic.

### Formula #110

(a) one, two, three, 4, 5, 6, 7, 8, 9, 10 or all of the following terepenes: Bisabolene; alpha-Farnesene; Humulene; Nerolidol; cis-Nerolidol; Camphor; Linalool; Camphene; alpha-Pinene; Terpinolene; Fenchone; beta-Pinene; Fenchol; Terpineol; Myrcene; Borneol; D-Limonene; BetaCaryophyllene; beta-Farnesene;

Optionally formulated as: Bisabolene 1.52 (mg/g), alpha-Farnesene 0.45 (mg/g), Humulene 1.13 (mg/g), Nerolidol 0.15 (mg/g), cis-Nerolidol 0.19 (mg/g), Camphor 0.01 (mg/g), Linalool 1.45 (mg/g), Camphene 0.06 (mg/g), alpha-Pinene 0.21 (mg/g), Terpinolene 0.05 (mg/g), Fenchone 0.03 (mg/g), beta-Pinene 0.37 (mg/g), Fenchol 0.32 (mg/g), Terpineol 0.27 (mg/g), Myrcene 1.92 (mg/g), Borneol 0.06 (mg/g), D-Limonene 2.16 (mg/g), BetaCaryophyllene 4.34 (mg/g), beta-Farnesene 0.78 (mg/g),

(b) in alternative embodiment, this formulation is used to treat, ameliorate, lesson the symptoms of, or prevent any one of the following conditions, or be used as an: Anti-inflammatory, Anti-bacterial, Anti-cancer, Appetite Suppressant, Anti-insomnia, Anti-depressant, Anti-convulsive, Analgesic,

(c) in alternative embodiment, this formulation is used to treat, ameliorate, lesson the symptoms of, or prevent any one of the following conditions, or be used as an: Anti-spasmotic, Neuroprotective, Anti-ischemic, Bronchodilator, Appetite Stimulant, Anti-psychotic, Anti-anxiety (Anxiolytic), Immunosuppressive, Anti-emetic.

### Formula #111

(a) one, two, three, 4, 5, 6, 7, 8, 9, 10 or all of the following terepenes: Camphor; Linalool; alpha-Phellandrene; gamma-Terpinene; alpha-Terpinene; 4-Carvomenthenol; Terpinolene; Fenchol; Terpineol; 3-Carene; Borneol; β-Eudesmol; Guaiol; D-Limonene; alpha-Bisabolol; Bisabolene; BetaCaryophyllene; alpha-Farnesene; beta-Farnesene; Humulene; beta-Ocimene; Nerolidol; alpha-Pinene; beta-Pinene; Sabinene; Myrcene;

Optionally formulated as: Camphor 0.04 (mg/g), Linalool 0.22 (mg/g), alpha-Phellandrene 0.28 (mg/g), gamma-Terpinene 0.12 (mg/g), alpha-Terpinene 0.15 (mg/g), 4-Carvomenthenol 0.07 (mg/g), Terpinolene 4.01 (mg/g), Fenchol 0.15 (mg/g), Terpineol 0.21 (mg/g), 3-Carene 0.2 (mg/g), Borneol 0.06 (mg/g), (3-Eudesmol 0.42 (mg/g), Guaiol 0.41 (mg/g), D-Limonene 0.48 (mg/g), alpha-Bisabolol 0.08 (mg/g), Bisabolene 1.04 (mg/g), BetaCaryophyllene 0.94 (mg/g), alpha-Farnesene 0.67 (mg/g), beta-Farnesene 0.11 (mg/g), Humulene 0.28 (mg/g), beta-Ocimene 0.62 (mg/g), Nerolidol 0.11 (mg/g), alpha-Pinene 0.45 (mg/g), beta-Pinene 0.64 (mg/g), Sabinene 0.08 (mg/g), Myrcene 0.89 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-cancer, Anti-inflammatory, Appetite Suppressant,

(c) in alternative embodiment, this formulation is used to treat, ameliorate, lesson the symptoms of, or prevent any one of the following conditions, or be used as an: Anti-anxiety (Anxiolytic), Neuroprotective, Anti-emetic, Anti-spasmotic, Bronchodilator, Anti-psychotic, Immunosuppressive, Appetite Stimulant, Anti-ischemic.

### Formula #112

(a) one, two, three, 4, 5, 6, 7, 8, 9, 10 or all of the following terepenes: alpha-Bisabolol; Camphor; Linalool; Camphene; alpha-Pinene; Fenchone; beta-Pinene; Fenchol; Terpineol; Myrcene; Borneol; D-Limonene; Bisabolene; BetaCaryophyllene; alpha-Farnesene; Humulene; beta-Ocimene; Nerolidol; cis-Nerolidol;

Optionally formulated as: alpha-Bisabolol 0.09 (mg/g), Camphor 0.01 (mg/g), Linalool 2.24 (mg/g), Camphene 0.17 (mg/g), alpha-Pinene 1.25 (mg/g), Fenchone 0.05 (mg/g), beta-Pinene 1.08 (mg/g), Fenchol 0.65 (mg/g), Terpineol 0.47 (mg/g), Myrcene 0.26 (mg/g), Borneol 0.11 (mg/g), D-Limonene 4.6 (mg/g), Bisabolene 0.58 (mg/g), BetaCaryophyllene 0.62 (mg/g), alpha-Farnesene 0.61 (mg/g), Humulene 0.23 (mg/g), beta-Ocimene 0.14 (mg/g), Nerolidol 0.09 (mg/g), cis-Nerolidol 0.08 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-psychotic, Anti-anxiety (Anxiolytic), Anti-ischemic, Neuroprotective, Anti-spasmotic, Immunosuppressive, Bronchodilator, Anti-emetic.

### Formula #113

(a) one, two, three, 4, 5, 6, 7, 8, 9, 10 or all of the following terepenes: Linalool; Fenchol; Borneol; D-Limonene; alpha-Bisabolol; Caryophyllene oxide; Bisabolene; Phytol; BetaCaryophyllene; beta-Farnesene; Humulene; Nerolidol; cis-Nerolidol; Camphene; alpha-Pinene; beta-Pinene; Myrcene;

Optionally formulated as: Linalool 0.28 (mg/g), Fenchol 0.06 (mg/g), Borneol 0.03 (mg/g), D-Limonene 0.23 (mg/g), alpha-Bisabolol 0.11 (mg/g), Caryophyllene oxide 0.04 (mg/g), Bisabolene 0.47 (mg/g), Phytol 0.1 (mg/g), BetaCaryophyllene 1.6 (mg/g), beta-Farnesene 0.15 (mg/g), Humulene 0.54 (mg/g), Nerolidol 0.05 (mg/g), cis-Nerolidol 0.05 (mg/g), Camphene 0.04 (mg/g), alpha-Pinene 1.86 (mg/g), beta-Pinene 0.73 (mg/g), Myrcene 1.81 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-cancer, Anti-inflammatory, Appetite Suppressant,

(c) in alternative embodiment, this formulation is used to treat, ameliorate, lesson the symptoms of, or prevent any one of the following conditions, or be used as an: Neuroprotective, Anti-ischemic, Immunosuppressive, Anti-anxiety (Anxiolytic), Anti-spasmotic, Anti-psychotic, Appetite Stimulant, Anti-emetic, Bronchodilator.

### Formula #114

(a) one, two, three, 4, 5, 6, 7, 8, 9, 10 or all of the following terepenes: Bisabolene; BetaCaryophyllene; alpha-Farnesene; Humulene; Nerolidol; cis-Nerolidol; Linalool; alpha-Pinene; beta-Pinene; Fenchol; Myrcene; Borneol; D-Limonene; Geranyl Acetate;

Optionally formulated as: Bisabolene 0.65 (mg/g), BetaCaryophyllene 0.68 (mg/g), alpha-Farnesene 0.9 (mg/g), Humulene 0.27 (mg/g), Nerolidol 0.09 (mg/g), cis-Nerolidol 0.07 (mg/g), Linalool 0.92 (mg/g), alpha-Pinene 0.1 (mg/g), beta-Pinene 0.13 (mg/g), Fenchol 0.07 (mg/g), Myrcene 0.05 (mg/g), Borneol 0.02 (mg/g), D-Limonene 1.07 (mg/g), Geranyl Acetate 0.02 (mg/g),

(b) in alternative embodiment, this formulation is used to treat, ameliorate, lesson the symptoms of, or prevent any one of the following conditions, or be used as an: Anti-inflammatory, Anti-bacterial, Anti-cancer, Appetite Suppressant, Anti-insomnia, Anti-depressant, Anti-convulsive, Analgesic,

(c) in alternative embodiment, this formulation is used to treat, ameliorate, lesson the symptoms of, or prevent any one of the following conditions, or be used as an: Anti-psychotic, Bronchodilator, Immunosuppressive, Anti-spasmotic, Anti-anxiety (Anxiolytic), Anti-emetic, Appetite Stimulant, Neuroprotective, Anti-ischemic.

### Formula #115

(a) one, two, three, 4, 5, 6, 7, 8, 9, 10 or all of the following terepenes: β-Eudesmol; Guaiol; alpha-Bisabolol; Bisabolene; Linalool; alpha-Pinene; alpha-Phellandrene; gamma-Terpinene; alpha-Terpinene; 4-Carvomenthenol; Terpinolene; beta-Pinene; Fenchol; Sabinene; 3-Carene; Myrcene; D-Menthol; D-Limonene; BetaCaryophyllene; alpha-Farnesene; Humulene; beta-Ocimene;

Optionally formulated as: β-Eudesmol 0.62 (mg/g), Guaiol 0.44 (mg/g), alpha-Bisabolol 0.11 (mg/g), Bisabolene 0.54 (mg/g), Linalool 0.1 (mg/g), alpha-Pinene 0.45 (mg/g), alpha-Phellandrene 0.37 (mg/g), gamma-Terpinene 0.12 (mg/g), alpha-Terpinene 0.22 (mg/g), 4-Carvomenthenol 0.05 (mg/g), Terpinolene 5.12 (mg/g), beta-Pinene 0.64 (mg/g), Fenchol 0.03 (mg/g), Sabinene 0.07 (mg/g), 3-Carene 0.25 (mg/g), Myrcene 3.99 (mg/g), D-Menthol 0.06 (mg/g), D-Limonene 0.27 (mg/g), BetaCaryophyllene 1.08 (mg/g), alpha-Farnesene 0.17 (mg/g), Humulene 0.27 (mg/g), beta-Ocimene 1.42 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Neuroprotective, Anti-ischemic, Immunosuppressive, Appetite Stimulant, Anti-emetic, Anti-psychotic, Anti-spasmotic, Bronchodilator.

### Formula #116

(a) one, two, three, 4, 5, 6, 7, 8, 9, 10 or all of the following terepenes: alpha-Bisabolol; Caryophyllene oxide; Linalool; Camphene; alpha-Pinene; Fenchone; beta-Pinene; Fenchol; Terpineol; Myrcene; Borneol; D-Limonene; Bisabolene; BetaCaryophyllene; Humulene; beta-Ocimene; Nerolidol; cis-Nerolidol;

Optionally formulated as: alpha-Bisabolol 0.14 (mg/g), Caryophyllene oxide 0.03 (mg/g), Linalool 1 (mg/g), Camphene 0.04 (mg/g), alpha-Pinene 0.14 (mg/g), Fenchone 0.05 (mg/g), beta-Pinene 0.29 (mg/g), Fenchol 0.2 (mg/g), Terpineol 0.17 (mg/g), Myrcene 4.91 (mg/g), Borneol 0.06 (mg/g), D-Limonene 0.98 (mg/g), Bisabolene 0.61 (mg/g), BetaCaryophyllene 1.64 (mg/g), Humulene 0.39 (mg/g), beta-Ocimene 0.09 (mg/g), Nerolidol 0.07 (mg/g), cis-Nerolidol 0.05 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-psychotic, Anti-anxiety (Anxiolytic), Anti-spasmotic, Anti-ischemic, Anti-emetic, Bronchodilator, Neuroprotective, Immunosuppressive.

### Formula #117

(a) one, two, three, 4, 5, 6, 7, 8, 9, 10 or all of the following terepenes: Linalool; Camphene; beta-Pinene; Fenchol; Terpineol; Myrcene; Borneol; D-Limonene; Bisabolene; BetaCaryophyllene; alpha-Farnesene; Humulene; beta-Ocimene; cis-Nerolidol; alpha-Pinene;

Optionally formulated as: Linalool 0.49 (mg/g), Camphene 0.08 (mg/g), beta-Pinene 0.44 (mg/g), Fenchol 0.3 (mg/g), Terpineol 0.16 (mg/g), Myrcene 0.2 (mg/g), Borneol 0.06 (mg/g), D-Limonene 1.45 (mg/g), Bisabolene 0.99 (mg/g), BetaCaryophyllene 1.8 (mg/g), alpha-Farnesene 0.47 (mg/g), Humulene 0.43 (mg/g), beta-Ocimene 0.15 (mg/g), cis-Nerolidol 0.06 (mg/g), alpha-Pinene 0.54 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-inflammatory, Anti-cancer, Appetite Suppressant,

(c) in alternative embodiment, this formulation is used to treat, ameliorate, lesson the symptoms of, or prevent any one of the following conditions, or be used as an: Anti-emetic, Anti-psychotic, Neuroprotective, Anti-ischemic, Appetite Stimulant, Anti-anxiety (Anxiolytic), Anti-spasmotic, Immunosuppressive, Bronchodilator.

### Formula #118

(a) one, two, three, 4, 5, 6, 7, 8, 9, 10 or all of the following terepenes: Linalool; alpha-Pinene; alpha-Phellandrene; gamma-Terpinene; alpha-Terpinene; Terpinolene; beta-Pinene; Terpineol; Sabinene; Myrcene; beta-Ocimene; Nerolidol; cis-Nerolidol; alpha-Ocimene; Sabinene Hydrate; Camphor; Eucalyptol; Camphene; p-Cymene; cineole; Fenchol; 3-Carene; Borneol; D-Limonene; Caryophyllene oxide; Bisabolene; BetaCaryophyllene; alpha-Farnesene; beta-Farnesene; Humulene;

Optionally formulated as: Linalool 11.2 (mg/g), alpha-Pinene 37.8 (mg/g), alpha-Phellandrene 13.7 (mg/g), gamma-Terpinene 2.5 (mg/g), alpha-Terpinene 5.9 (mg/g), Terpinolene 272 (mg/g), beta-Pinene 35.7 (mg/g), Terpineol 2.6 (mg/g), Sabinene 0.9 (mg/g), Myrcene 175 (mg/g), beta-Ocimene 66.7 (mg/g), Nerolidol 0.3 (mg/g), cis-Nerolidol 0.8 (mg/g), alpha-Ocimene 0.5 (mg/g), Sabinene Hydrate 0.6 (mg/g), Camphor 1 (mg/g), Eucalyptol 1.4 (mg/g), Camphene 1.4 (mg/g), p-Cymene 1.3 (mg/g), cineole 1.7 (mg/g), Fenchol 3.9 (mg/g), 3-Carene 8.8 (mg/g), Borneol 0.5 (mg/g), D-Limonene 57.6 (mg/g), Caryophyllene oxide 0.6 (mg/g), Bisabolene 11 (mg/g), BetaCaryophyllene 31.7 (mg/g), alpha-Farnesene 7.3 (mg/g), beta-Farnesene 3.7 (mg/g), Humulene 6.3 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Bronchodilator, Immunosuppressive, Anti-emetic, Anti-spasmotic, Anti-anxiety (Anxiolytic), Neuroprotective, Anti-psychotic, Anti-ischemic

### Formula #119

(a) one, two, three, 4, 5, 6, 7, 8, 9, 10 or all of the following terepenes: Linalool; alpha-Pinene; alpha-Phellandrene; gamma-Terpinene; alpha-Terpinene; Terpinolene; beta-Pinene; Terpineol; Sabinene; Myrcene; beta-Ocimene; cis-Nerolidol; alpha-Ocimene; Valencene; Sabinene Hydrate; Camphor; Eucalyptol; Camphene; p-Cymene; cineole; Fenchone; Fenchol; 3-Carene; Borneol; D-Limonene; Caryophyllene oxide; Bisabolene; BetaCaryophyllene; alpha-Farnesene; beta-Farnesene; Humulene;

Optionally formulated as: Linalool 18.9 (mg/g), alpha-Pinene 17 (mg/g), alpha-Phellandrene 9.8 (mg/g), gamma-Terpinene 1.6 (mg/g), alpha-Terpinene 3.9 (mg/g), Terpinolene 212.5 (mg/g), beta-Pinene 30.5 (mg/g), Terpineol 4 (mg/g), Sabinene 1.2 (mg/g), Myrcene 104.7 (mg/g), beta-Ocimene 53.8 (mg/g), cis-Nerolidol 0.4 (mg/g), alpha-Ocimene 0.5 (mg/g), Valencene 1.3 (mg/g), Sabinene Hydrate 0.5 (mg/g), Camphor 3.7 (mg/g), Eucalyptol 1.2 (mg/g), Camphene 1.4 (mg/g), p-Cymene 3.9 (mg/g), cineole 0.9 (mg/g), Fenchone 1.5 (mg/g), Fenchol 4.5 (mg/g), 3-Carene 9.9 (mg/g), Borneol 0.6 (mg/g), D-Limonene 67.4 (mg/g), Caryophyllene oxide 1.8 (mg/g), Bisabolene 7.6 (mg/g), BetaCaryophyllene 19.2 (mg/g), alpha-Farnesene 4 (mg/g), beta-Farnesene 2.3 (mg/g), Humulene 4.2 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Appetite Stimulant, Anti-anxiety (Anxiolytic), Immunosuppressive, Anti-psychotic, Anti-spasmotic, Anti-ischemic, Anti-emetic, Neuroprotective

### Formula #120

(a) one, two, three, 4, 5, 6, 7, 8, 9, 10 or all of the following terepenes: 3-Carene; Eucalyptol; Linalool; Valencene; alpha-Pinene; alpha-Phellandrene; gamma-Terpinene; alpha-Terpinene; p-Cymene; cineole; Terpinolene; Fenchone; beta-Pinene; Fenchol; Terpineol; Sabinene; Sabinene Hydrate; Myrcene; D-Limonene; Caryophyllene oxide; BetaCaryophyllene; alpha-Farnesene; beta-Farnesene; Humulene; beta-Ocimene; Nerolidol; cis-Nerolidol; alpha-Ocimene; Camphene; alpha-Bisabolol; Borneol; Bisabolene; Camphor;

Optionally formulated as: 3-Carene 2.7 (mg/g), Eucalyptol 3.4 (mg/g), Linalool 14.4 (mg/g), Valencene 6.8 (mg/g), alpha-Pinene 21.1 (mg/g), alpha-Phellandrene 4 (mg/g), gamma-Terpinene 0.8 (mg/g), alpha-Terpinene 1.7 (mg/g), p-Cymene 0.3 (mg/g), cineole 0.4 (mg/g), Terpinolene 71.6 (mg/g), Fenchone 0.5 (mg/g), beta-Pinene 27.4 (mg/g), Fenchol 7.9 (mg/g), Terpineol 2.3 (mg/g), Sabinene 0.5 (mg/g), Sabinene Hydrate 0.5 (mg/g), Myrcene 97.9 (mg/g), D-Limonene 108 (mg/g), Caryophyllene oxide 0.9 (mg/g), BetaCaryophyllene 220.9 (mg/g), alpha-Farnesene 9.1 (mg/g), beta-Farnesene 10.9 (mg/g), Humulene 57.5 (mg/g), beta-Ocimene 18.8 (mg/g), Nerolidol 0.7 (mg/g), cis-Nerolidol 2.2 (mg/g), alpha-Ocimene 0.2 (mg/g), Camphene 2.6 (mg/g), alpha-Bisabolol 0.4 (mg/g), Borneol 0.8 (mg/g), Bisabolene 36.6 (mg/g), Camphor 0.1 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Immunosuppressive, Appetite Stimulant, Neuroprotective, Anti-anxiety (Anxiolytic), Anti-psychotic, Anti-emetic, Bronchodilator, Anti-spasmotic

### Formula #121

(a) one, two, three, 4, 5, 6, 7, 8, 9, 10 or all of the following terepenes: Linalool; alpha-Pinene; alpha-Phellandrene; gamma-Terpinene; alpha-Terpinene; Terpinolene; Fenchone; beta-Pinene; Fenchol; Terpineol; Myrcene; D-Limonene; alpha-Farnesene; beta-Farnesene; Humulene; beta-Ocimene; Nerolidol; cis-Nerolidol; alpha-Ocimene; Valencene; Sabinene Hydrate; Eucalyptol; Camphene; p-Cymene; 3-Carene; Borneol; Caryophyllene oxide; Bisabolene; BetaCaryophyllene;

Optionally formulated as: Linalool 17.1 (mg/g), alpha-Pinene 52.2 (mg/g), alpha-Phellandrene 0.5 (mg/g), gamma-Terpinene 0.4 (mg/g), alpha-Terpinene 0.2 (mg/g), Terpinolene 14.9 (mg/g), Fenchone 1.6 (mg/g), beta-Pinene 36.4 (mg/g), Fenchol 11.1 (mg/g), Terpineol 5 (mg/g), Myrcene 18.9 (mg/g), D-Limonene 197.6 (mg/g), alpha-Farnesene 9.6 (mg/g), beta-Farnesene 9.3 (mg/g), Humulene 14.6 (mg/g), beta-Ocimene 209.1 (mg/g), Nerolidol 0.5 (mg/g), cis-Nerolidol 1.5 (mg/g), alpha-Ocimene 4.4 (mg/g), Valencene 3.6 (mg/g), Sabinene Hydrate 1.1 (mg/g), Eucalyptol 3.6 (mg/g), Camphene 3.7 (mg/g), p-Cymene 0.4 (mg/g), 3-Carene 0.5 (mg/g), Borneol 1.4 (mg/g), Caryophyllene oxide 2.8 (mg/g), Bisabolene 26.6 (mg/g), BetaCaryophyllene 50.2 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-psychotic, Anti-ischemic, Anti-emetic, Bronchodilator, Anti-spasmotic, Anti-anxiety (Anxiolytic), Immunosuppressive, Neuroprotective

### Formula #122

(a) one, two, three, 4, 5, 6, 7, 8, 9, 10 or all of the following terepenes: Camphor; Linalool; Camphene; alpha-Pinene; Terpinolene; Fenchone; beta-Pinene; Fenchol; Terpineol; Myrcene; Borneol; D-Limonene; Caryophyllene oxide; Bisabolene; BetaCaryophyllene; beta-Farnesene; Humulene; beta-Ocimene; Valencene;

Optionally formulated as: Camphor 0.2 (mg/g), Linalool 10.4 (mg/g), Camphene 3.7 (mg/g), alpha-Pinene 120.2 (mg/g), Terpinolene 0.7 (mg/g), Fenchone 0.9 (mg/g), beta-Pinene 63.8 (mg/g), Fenchol 6.2 (mg/g), Terpineol 1.6 (mg/g), Myrcene 466 (mg/g), Borneol 0.7 (mg/g), D-Limonene 137.1 (mg/g), Caryophyllene oxide 1.1 (mg/g), Bisabolene 3.7 (mg/g), BetaCaryophyllene 24.6 (mg/g), beta-Farnesene 1.8 (mg/g), Humulene 4.5 (mg/g), beta-Ocimene 5.6 (mg/g), Valencene 0.8 (mg/g), (b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Neuroprotective, Anti-anxiety (Anxiolytic), Appetite Stimulant, Anti-spasmotic, Bronchodilator, Anti-ischemic, Anti-emetic, Anti-psychotic

### Formula #123

(a) one, two, three, 4, 5, 6, 7, 8, 9, 10 or all of the following terepenes: Sabinene Hydrate; Terpinolene; Terpineol; beta-Pinene; alpha-Pinene; alpha-Phellandrene; gamma-Terpinene; alpha-Terpinene; Sabinene; 4-Carvomenthenol; beta-Ocimene; Nerolidol; cis-Nerolidol; alpha-Ocimene; Valencene; Fenchol; Camphor; Myrcene; Borneol; D-Menthol; D-Limonene; Caryophyllene oxide; Bisabolene; BetaCaryophyllene; alpha-Farnesene; beta-Farnesene; Humulene; Eucalyptol; Linalool; Camphene; p-Cymene; Fenchone; 3-Carene;

Optionally formulated as: Sabinene Hydrate 1.1 (mg/g), Terpinolene 13.1 (mg/g), Terpineol 2.5 (mg/g), beta-Pinene 26.4 (mg/g), alpha-Pinene 14.2 (mg/g), alpha-Phellandrene 0.4 (mg/g), gamma-Terpinene 0.4 (mg/g), alpha-Terpinene 0.3 (mg/g), Sabinene 0.2 (mg/g), 4-Carvomenthenol 0.2 (mg/g), beta-Ocimene 1.3 (mg/g), Nerolidol 0.8 (mg/g), cis-Nerolidol 2.8 (mg/g), alpha-Ocimene 0.3 (mg/g), Valencene 8.6 (mg/g), Fenchol 11 (mg/g), Camphor 0.2 (mg/g), Myrcene 29 (mg/g), Borneol 1.1 (mg/g), D-Menthol 0.3 (mg/g), D-Limonene 198.6 (mg/g), Caryophyllene oxide 1 (mg/g), Bisabolene 41.9 (mg/g), BetaCaryophyllene 216 (mg/g), alpha-Farnesene 9.8 (mg/g), beta-Farnesene 19.5 (mg/g), Humulene 60.3 (mg/g), Eucalyptol 8 (mg/g), Linalool 42.8 (mg/g), Camphene 3.8 (mg/g), p-Cymene 0.2 (mg/g), Fenchone 1.7 (mg/g), 3-Carene 0.3 (mg/g),

(b) in alternative embodiment, this formulation is used to treat, ameliorate, lesson the symptoms of, or prevent any one of the following conditions, or be used as an: Anti-bacterial, Anti-insomnia, Anti-inflammatory, Analgesic, Anti-cancer, Anti-depressant, Appetite Suppressant, Anti-convulsive,

(c) in alternative embodiment, this formulation is used to treat, ameliorate, lesson the symptoms of, or prevent any one of the following conditions, or be used as an: Anti-psychotic, Appetite Stimulant, Anti-spasmotic, Anti-anxiety (Anxiolytic), Immunosuppressive, Anti-ischemic, Neuroprotective, Bronchodilator, Anti-emetic

### Formula #124

(a) one, two, three, 4, 5, 6, 7, 8, 9, 10 or all of the following terepenes: beta-Elemene; Linalool; alpha-Pinene; alpha-Phellandrene; gamma-Terpinene; alpha-Terpinene; Terpinolene; beta-Pinene; Fenchol; Terpineol; Myrcene; β-Eudesmol; Guaiol; D-Limonene; BetaCaryophyllene; Humulene; beta-Ocimene; 3-Carene;

Optionally formulated as: beta-Elemene 0.1 (mg/g), Linalool 0.51 (mg/g), alpha-Pinene 0.5 (mg/g), alpha-Phellandrene 0.3 (mg/g), gamma-Terpinene 0.06 (mg/g), alpha-Terpinene 0.13 (mg/g), Terpinolene 3.7 (mg/g), beta-Pinene 0.73 (mg/g), Fenchol 0.09 (mg/g), Terpineol 0.13 (mg/g), Myrcene 2.85 (mg/g), β-Eudesmol 0.06 (mg/g), Guaiol 0.23 (mg/g), D-Limonene 0.49 (mg/g), BetaCaryophyllene 0.61 (mg/g), Humulene 0.15 (mg/g), beta-Ocimene 2.32 (mg/g), 3-Carene 0.23 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Appetite Stimulant, Neuroprotective, Anti-psychotic, Anti-anxiety (Anxiolytic), Anti-spasmotic, Anti-emetic, Bronchodilator, Anti-ischemic

### Formula #125

(a) one, two, three, 4, 5, 6, 7, 8, 9, 10 or all of the following terepenes: Linalool; Camphene; alpha-Pinene; beta-Pinene; Fenchol; Myrcene; Borneol; D-Limonene; BetaCaryophyllene; Humulene;

Optionally formulated as: Linalool 0.56 (mg/g), Camphene 0.1 (mg/g), alpha-Pinene 0.3 (mg/g), beta-Pinene 0.54 (mg/g), Fenchol 0.25 (mg/g), Myrcene 3.14 (mg/g), Borneol 0.05 (mg/g), D-Limonene 1.72 (mg/g), BetaCaryophyllene 3.66 (mg/g), Humulene 0.66 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Immunosuppressive, Anti-spasmotic, Appetite Stimulant, Anti-psychotic, Bronchodilator, Anti-anxiety (Anxiolytic), Anti-emetic, Anti-ischemic.

### Formula #126

(a) one, two, three, 4, 5, 6, 7, 8, 9, 10 or all of the following terepenes: Linalool; Camphene; alpha-Pinene; Terpinolene; Fenchone; beta-Pinene; Fenchol; Terpineol; Myrcene; Borneol; Guaiol; D-Limonene; BetaCaryophyllene; Humulene; beta-Ocimene; beta-Elemene;

Optionally formulated as: Linalool 0.77 (mg/g), Camphene 0.17 (mg/g), alpha-Pinene 0.66 (mg/g), Terpinolene 0.02 (mg/g), Fenchone 0.09 (mg/g), beta-Pinene 0.9 (mg/g), Fenchol 0.52 (mg/g), Terpineol 0.25 (mg/g), Myrcene 0.4 (mg/g), Borneol 0.07 (mg/g), Guaiol 0.36 (mg/g), D-Limonene 3.82 (mg/g), BetaCaryophyllene 1.03 (mg/g), Humulene 0.24 (mg/g), beta-Ocimene 0.92 (mg/g), beta-Elemene 0.13 (mg/g),

(b) in alternative embodiment, this formulation is used to treat, ameliorate, lesson the symptoms of, or prevent any one of the following conditions, or be used as an:

Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Immunosuppressive, Appetite Stimulant, Neuroprotective, Anti-psychotic, Anti-emetic, Anti-ischemic, Anti-anxiety (Anxiolytic), Bronchodilator

### Formula #127

(a) one, two, three, 4, 5, 6, 7, 8, 9, 10 or all of the following terepenes: Linalool; alpha-Pinene; beta-Pinene; Terpineol; Myrcene; D-Limonene; cis-Nerolidol; Camphene; Fenchone; Fenchol; Borneol; BetaCaryophyllene; Humulene; beta-Elemene;

Optionally formulated as: Linalool 1.15 (mg/g), alpha-Pinene 2.21 (mg/g), beta-Pinene 1.38 (mg/g), Terpineol 0.24 (mg/g), Myrcene 1.29 (mg/g), D-Limonene 2.86 (mg/g), cis-Nerolidol 0.14 (mg/g), Camphene 0.17 (mg/g), Fenchone 0.1 (mg/g), Fenchol 0.43 (mg/g), Borneol 0.1 (mg/g), BetaCaryophyllene 4.23 (mg/g), Humulene 0.73 (mg/g), beta-Elemene 0.08 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Anti-emetic, Bronchodilator, Anti-psychotic, Immunosuppressive, Anti-ischemic, Anti-spasmotic, Appetite Stimulant, Anti-anxiety (Anxiolytic).

### Formula #128

(a) one, two, three, 4, 5, 6, 7, 8, 9, 10 or all of the following terepenes: Linalool; alpha-Pinene; beta-Pinene; Fenchol; Myrcene; Guaiol; D-Limonene; Humulene; Camphene; β-Eudesmol; BetaCaryophyllene; beta-Elemene;

Optionally formulated as: Linalool 1.06 (mg/g), alpha-Pinene 0.14 (mg/g), beta-Pinene 0.21 (mg/g), Fenchol 0.1 (mg/g), Myrcene 2.13 (mg/g), Guaiol 0.5 (mg/g), D-Limonene 1.01 (mg/g), Humulene 0.11 (mg/g), Camphene 0.04 (mg/g), β-Eudesmol 0.09 (mg/g), BetaCaryophyllene 0.52 (mg/g), beta-Elemene 0.13 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-psychotic, Immunosuppressive, Anti-spasmotic, Neuroprotective, Appetite Stimulant, Bronchodilator, Anti-ischemic, Anti-anxiety (Anxiolytic)

### Formula #129

(a) one, two, three, 4, 5, 6, 7, 8, 9, 10 or all of the following terepenes: Linalool; alpha-Pinene; Terpinolene; beta-Pinene; Myrcene; beta-Ocimene; Camphene; Fenchone; Fenchol; Borneol; D-Limonene; BetaCaryophyllene; alpha-Farnesene; Humulene; beta-Elemene;

Optionally formulated as: Linalool 1.38 (mg/g), alpha-Pinene 0.42 (mg/g), Terpinolene 0.02 (mg/g), beta-Pinene 0.59 (mg/g), Myrcene 0.38 (mg/g), beta-Ocimene 0.87 (mg/g), Camphene 0.11 (mg/g), Fenchone 0.07 (mg/g), Fenchol 0.41 (mg/g), Borneol 0.06 (mg/g), D-Limonene 2.67 (mg/g), BetaCaryophyllene 1.39 (mg/g), alpha-Farnesene 3.48 (mg/g), Humulene 0.38 (mg/g), beta-Elemene 0.08 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Anti-psychotic, Immunosuppressive, Bronchodilator, Anti-spasmotic, Neuroprotective, Appetite Stimulant, Anti-ischemic, Anti-emetic.

### Formula #130

(a) one, two, three, 4, 5, 6, 7, 8, 9, 10 or all of the following terepenes: alpha-Pinene; Terpinolene; beta-Pinene; Myrcene; Valencene; Linalool; Camphene; Fenchone; Fenchol; Borneol; D-Limonene; BetaCaryophyllene; Humulene; beta-Elemene;

Optionally formulated as: alpha-Pinene 0.4 (mg/g), Terpinolene 0.05 (mg/g), beta-Pinene 0.61 (mg/g), Myrcene 0.69 (mg/g), Valencene 0.07 (mg/g), Linalool 2.18 (mg/g), Camphene 0.12 (mg/g), Fenchone 0.06 (mg/g), Fenchol 0.31 (mg/g), Borneol 0.06 (mg/g), D-Limonene 3.62 (mg/g), BetaCaryophyllene 1.76 (mg/g), Humulene 0.34 (mg/g), beta-Elemene 0.08 (mg/g),

(b) in alternative embodiment, this formulation is used to treat, ameliorate, lesson the symptoms of, or prevent any one of the following conditions, or be used as an: Anti-inflammatory, Anti-bacterial, Anti-insomnia, Analgesic, Anti-cancer, Anti-depressant, Anti-convulsive, Appetite Suppressant,

(c) in alternative embodiment, this formulation is used to treat, ameliorate, lesson the symptoms of, or prevent any one of the following conditions, or be used as an: Immunosuppressive, Anti-ischemic, Anti-anxiety (Anxiolytic), Anti-spasmotic, Neuroprotective, Anti-emetic, Anti-psychotic, Bronchodilator, Appetite Stimulant

### Formula #131

(a) one, two, three, 4, 5, 6, 7, 8, 9, 10 or all of the following terepenes: Valencene; Linalool; Camphene; alpha-Pinene; beta-Pinene; Fenchol; Myrcene; Borneol; D-Limonene; alpha-Bisabolol; BetaCaryophyllene; Humulene; cis-Nerolidol;

Optionally formulated as: Valencene 0.2 (mg/g), Linalool 0.93 (mg/g), Camphene 0.06 (mg/g), alpha-Pinene 0.22 (mg/g), beta-Pinene 0.42 (mg/g), Fenchol 0.37 (mg/g), Myrcene 1.22 (mg/g), Borneol 0.07 (mg/g), D-Limonene 2.11 (mg/g), alpha-Bisabolol 0.42 (mg/g), BetaCaryophyllene 4.08 (mg/g), Humulene 0.78 (mg/g), cis-Nerolidol 0.11 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Anti-ischemic, Bronchodilator, Anti-spasmotic, Appetite Stimulant, Immunosuppressive, Neuroprotective, Anti-emetic, Anti-anxiety (Anxiolytic).

### Formula #132

(a) one, two, three, 4, 5, 6, 7, 8, 9, 10 or all of the following terepenes: alpha-Pinene; Terpinolene; beta-Pinene; Valencene; Linalool; Camphene; Fenchol; Myrcene; Borneol; D-Limonene; BetaCaryophyllene; Humulene; cis-Nerolidol; beta-Elemene;

Optionally formulated as: alpha-Pinene 0.35 (mg/g), Terpinolene 0.03 (mg/g), beta-Pinene 0.61 (mg/g), Valencene 0.09 (mg/g), Linalool 2.89 (mg/g), Camphene 0.11 (mg/g), Fenchol 0.34 (mg/g), Myrcene 1.78 (mg/g), Borneol 0.05 (mg/g), D-Limonene 3.29 (mg/g), BetaCaryophyllene 1.92 (mg/g), Humulene 0.39 (mg/g), cis-Nerolidol 0.03 (mg/g), beta-Elemene 0.13 (mg/g),

(b) in alternative embodiment, this formulation is used to treat, ameliorate, lesson the symptoms of, or prevent any one of the following conditions, or be used as an: Anti-inflammatory, Anti-bacterial, Anti-insomnia, Anti-depressant, Anti-convulsive, Analgesic, Anti-cancer, Appetite Suppressant,

(c) in alternative embodiment, this formulation is used to treat, ameliorate, lesson the symptoms of, or prevent any one of the following conditions, or be used as an: Neuroprotective, Appetite Stimulant, Anti-anxiety (Anxiolytic), Immunosuppressive, Anti-emetic, Anti-spasmotic, Bronchodilator, Anti-psychotic, Anti-ischemic.

### Formula #133

(a) one, two, three, 4, 5, 6, 7, 8, 9, 10 or all of the following terepenes: Valencene; Linalool; Camphene; alpha-Pinene; Fenchone; beta-Pinene; Fenchol; Terpineol; Myrcene; Borneol; D-Limonene; alpha-Bisabolol; Caryophyllene oxide; Bisabolene; BetaCaryophyllene; alpha-Farnesene; beta-Farnesene; Humulene; cis-Nerolidol;

Optionally formulated as: Valencene 0.22 (mg/g), Linalool 1.88 (mg/g), Camphene 0.1 (mg/g), alpha-Pinene 0.33 (mg/g), Fenchone 0.05 (mg/g), beta-Pinene 0.54 (mg/g), Fenchol 0.53 (mg/g), Terpineol 0.38 (mg/g), Myrcene 2.15 (mg/g), Borneol 0.11 (mg/g), D-Limonene 2.3 (mg/g), alpha-Bisabolol 0.44 (mg/g), Caryophyllene oxide 0.06 (mg/g), Bisabolene 3.5 (mg/g), BetaCaryophyllene 3.16 (mg/g), alpha-Farnesene 0.82 (mg/g), beta-Farnesene 0.08 (mg/g), Humulene 0.76 (mg/g), cis-Nerolidol 0.21 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-anxiety (Anxiolytic), Immunosuppressive, Neuroprotective, Anti-emetic, Anti-psychotic, Anti-ischemic, Bronchodilator, Appetite Stimulant.

### Formula #134

(a) one, two, three, 4, 5, 6, 7, 8, 9, 10 or all of the following terepenes: Linalool; alpha-Pinene; Citronellol; beta-Pinene; Fenchol; Myrcene; D-Limonene; Caryophyllene oxide; Phytol; BetaCaryophyllene; beta-Farnesene; Humulene; beta-Ocimene; cis-Nerolidol; Camphene; Borneol; Bisabolene;

Optionally formulated as: Linalool 0.7 (mg/g), alpha-Pinene 4.23 (mg/g), Citronellol 0.08 (mg/g), beta-Pinene 1 (mg/g), Fenchol 0.12 (mg/g), Myrcene 10.64 (mg/g), D-Limonene 0.72 (mg/g), Caryophyllene oxide 0.05 (mg/g), Phytol 0.42 (mg/g), BetaCaryophyllene 1.54 (mg/g), beta-Farnesene 0.06 (mg/g), Humulene 0.37 (mg/g), beta-Ocimene 1.4 (mg/g), cis-Nerolidol 0.11 (mg/g), Camphene 0.08 (mg/g), Borneol 0.06 (mg/g), Bisabolene 1.45 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Immunosuppressive, Anti-ischemic, Anti-psychotic, Anti-emetic, Anti-anxiety (Anxiolytic), Anti-spasmotic, Appetite Stimulant, Bronchodilator, Neuroprotective.

### Formula #135

(a) one, two, three, 4, 5, 6, 7, 8, 9, 10 or all of the following terepenes: Linalool; Camphene; alpha-Pinene; 4-Carvomenthenol; Terpinolene; Fenchone; beta-Pinene; Fenchol; Terpineol; Myrcene; Borneol; β-Eudesmol; D-Menthol; D-Limonene; alpha-Bisabolol; Bisabolene; Phytol; BetaCaryophyllene; alpha-Farnesene; beta-Farnesene; Humulene; beta-Ocimene; Nerolidol; cis-Nerolidol; Valencene;

Optionally formulated as: Linalool 5.12 (mg/g), Camphene 0.51 (mg/g), alpha-Pinene 1.68 (mg/g), 4-Carvomenthenol 0.07 (mg/g), Terpinolene 0.1 (mg/g), Fenchone 0.34 (mg/g), beta-Pinene 2.14 (mg/g), Fenchol 4.21 (mg/g), Terpineol 1.33 (mg/g), Myrcene 2.18 (mg/g), Borneol 0.35 (mg/g), β-Eudesmol 0.19 (mg/g), D-Menthol 0.09 (mg/g), D-Limonene 9.19 (mg/g), alpha-Bisabolol 1.38 (mg/g), Bisabolene 8.05 (mg/g), Phytol 1.05 (mg/g), BetaCaryophyllene 20.92 (mg/g), alpha-Farnesene 2.15 (mg/g), beta-Farnesene 2.5 (mg/g), Humulene 4.93 (mg/g), beta-Ocimene 0.49 (mg/g), Nerolidol 0.67 (mg/g), cis-Nerolidol 0.35 (mg/g), Valencene 0.92 (mg/g), (b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Neuroprotective, Anti-spasmotic, Appetite Stimulant, Anti-psychotic, Anti-anxiety (Anxiolytic), Bronchodilator, Anti-ischemic, Anti-emetic.

### Formula #136

(a) one, two, three, 4, 5, 6, 7, 8, 9, 10 or all of the following terepenes: Linalool; 4-Carvomenthenol; Terpinolene; gamma-Terpineol; Fenchone; Fenchol; Borneol; Cedrol; Caryophyllene oxide; BetaCaryophyllene; alpha-Farnesene; beta-Farnesene; Humulene; Sabinene Hydrate; Camphene; alpha-Pinene; alpha-Phellandrene; gamma-Terpinene; p-Cymene; beta-Pinene; Limonene; 3-Carene; Myrcene; alpha-Ocimene; trans-beta-Ocimene;

Optionally formulated as: Linalool 483.63 (mg/g), 4-Carvomenthenol 5.96 (mg/g), Terpinolene 114.94 (mg/g), gamma-Terpineol 76.46 (mg/g), Fenchone 16.74 (mg/g), Fenchol 170.34 (mg/g), Borneol 8.37 (mg/g), Cedrol 0.84 (mg/g), Caryophyllene oxide 15.17 (mg/g), BetaCaryophyllene 850.08 (mg/g), alpha-Farnesene 75.15 (mg/g), beta-Farnesene 54.37 (mg/g), Humulene 193.74 (mg/g), Sabinene Hydrate 4.53 (mg/g), Camphene 63.63 (mg/g), alpha-Pinene 356.37 (mg/g), alpha-Phellandrene 4.97 (mg/g), gamma-Terpinene 1.57 (mg/g), p-Cymene 3 (mg/g), beta-Pinene 426.59 (mg/g), Limonene 3198.69 (mg/g), 3-Carene 3.96 (mg/g), Myrcene 2849.59 (mg/g), alpha-Ocimene 8.18 (mg/g), trans-beta-Ocimene 33.18 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-inflammatory, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-spasmotic, Neuroprotective, Appetite Stimulant, Anti-psychotic, Anti-emetic, Anti-anxiety (Anxiolytic), Bronchodilator, Anti-ischemic.

### Formula #137

(a) one, two, three, 4, 5, 6, 7, 8, 9, 10 or all of the following terepenes: Valencene; Camphor; Linalool; Citronellol; Fenchone; Fenchol; Terpineol; Myrcene; Borneol; Cedrol; β-Eudesmol; Guaiol; D-Limonene; alpha-Bisabolol; Caryophyllene oxide; Bisabolene; BetaCaryophyllene; alpha-Farnesene; beta-Farnesene; Humulene; Nerolidol; cis-Nerolidol;

Optionally formulated as: Valencene 12.04 (mg/g), Camphor 0.04 (mg/g), Linalool 13.54 (mg/g), Citronellol 0.77 (mg/g), Fenchone 0.59 (mg/g), Fenchol 3.31 (mg/g), Terpineol 4.91 (mg/g), Myrcene 1.32 (mg/g), Borneol 1.05 (mg/g), Cedrol 4.55 (mg/g), β-Eudesmol 46.88 (mg/g), Guaiol 199.65 (mg/g), D-Limonene 0.49 (mg/g), alpha-Bisabolol 224.34 (mg/g), Caryophyllene oxide 39.67 (mg/g), Bisabolene 51.6 (mg/g), BetaCaryophyllene 226.42 (mg/g), alpha-Farnesene 15.39 (mg/g), beta-Farnesene 28.72 (mg/g), Humulene 19.95 (mg/g), Nerolidol 47.27 (mg/g), cis-Nerolidol 6.68 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Anti-cancer, Analgesic, Appetite Suppressant,

(c) in alternative embodiment, this formulation is used to treat, ameliorate, lesson the symptoms of, or prevent any one of the following conditions, or be used as an: Neuroprotective, Appetite Stimulant, Anti-ischemic, Immunosuppressive, Anti-emetic, Anti-psychotic, Bronchodilator, Anti-anxiety (Anxiolytic), Anti-spasmotic.

### Formula #138

(a) one, two, three, 4, 5, 6, 7, 8, 9, 10 or all of the following terepenes: Linalool; alpha-Pinene; beta-Pinene; Myrcene; β-Eudesmol; Guaiol; D-Limonene; alpha-Bisabolol; Bisabolene; BetaCaryophyllene; Humulene; cis-Nerolidol; trans-beta-Ocimene;

Optionally formulated as: Linalool 0.6 (mg/g), alpha-Pinene 0.28 (mg/g), beta-Pinene 0.31 (mg/g), Myrcene 9.85 (mg/g), β-Eudesmol 0.45 (mg/g), Guaiol 2.09 (mg/g), D-Limonene 0.09 (mg/g), alpha-Bisabolol 0.35 (mg/g), Bisabolene 0.89 (mg/g), BetaCaryophyllene 1.77 (mg/g), Humulene 0.2 (mg/g), cis-Nerolidol 0.14 (mg/g), trans-beta-Ocimene 0.51 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Bronchodilator, Anti-psychotic, Anti-spasmotic, Appetite Stimulant, Anti-anxiety (Anxiolytic), Immunosuppressive, Neuroprotective, Anti-emetic.

### Formula #139

(a) one, two, three, 4, 5, 6, 7, 8, 9, 10 or all of the following terepenes: Linalool; alpha-Pinene; beta-Pinene; Myrcene; cis-Nerolidol; trans-beta-Ocimene; (3-Eudesmol; Guaiol; D-Limonene; alpha-Bisabolol; Bisabolene; BetaCaryophyllene; Humulene;

Optionally formulated as: Linalool 0.6 (mg/g), alpha-Pinene 0.28 (mg/g), beta-Pinene 0.31 (mg/g), Myrcene 9.85 (mg/g), cis-Nerolidol 0.14 (mg/g), trans-beta-Ocimene 0.51 (mg/g), β-Eudesmol 0.45 (mg/g), Guaiol 2.09 (mg/g), D-Limonene 0.09 (mg/g), alpha-Bisabolol 0.35 (mg/g), Bisabolene 0.89 (mg/g), BetaCaryophyllene 1.77 (mg/g), Humulene 0.2 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-spasmotic, Anti-emetic, Anti-anxiety (Anxiolytic), Anti-ischemic, Immunosuppressive, Neuroprotective, Bronchodilator, Anti-psychotic

### Formula #140

(a) one, two, three, 4, 5, 6, 7, 8, 9, 10 or all of the following terepenes: alpha-Pinene; beta-Pinene; Linalool; Myrcene; D-Limonene; alpha-Bisabolol; Caryophyllene oxide; Bisabolene; BetaCaryophyllene; Humulene; cis-Nerolidol;

Optionally formulated as: alpha-Pinene 0.07 (mg/g), beta-Pinene 0.22 (mg/g), Linalool 0.61 (mg/g), Myrcene 2.1 (mg/g), D-Limonene 0.58 (mg/g), alpha-Bisabolol 0.55 (mg/g), Caryophyllene oxide 0.07 (mg/g), Bisabolene 1.26 (mg/g), BetaCaryophyllene 2.81 (mg/g), Humulene 0.2 (mg/g), cis-Nerolidol 0.12 (mg/g), (b) in alternative embodiment, this formulation is used to treat, ameliorate, lesson the symptoms of, or prevent any one of the following conditions, or be used as an: Anti-inflammatory, Anti-bacterial, Anti-depressant, Anti-insomnia, Anti-convulsive, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-spasmotic, Anti-anxiety (Anxiolytic), Bronchodilator, Immunosuppressive, Anti-ischemic, Anti-emetic, Anti-psychotic, Neuroprotective.

### Formula #141

(a) one, two, three, 4, 5, 6, 7, 8, 9, 10 or all of the following terepenes: Linalool; alpha-Pinene; beta-Pinene; Terpineol; Myrcene; D-Limonene; Humulene; cis-Nerolidol; Camphene; Fenchol; Borneol; alpha-Bisabolol; Caryophyllene oxide; Bisabolene; BetaCaryophyllene; beta-Farnesene;

Optionally formulated as: Linalool 2.43 (mg/g), alpha-Pinene 0.18 (mg/g), beta-Pinene 0.7 (mg/g), Terpineol 0.21 (mg/g), Myrcene 3.64 (mg/g), D-Limonene 1.48 (mg/g), Humulene 0.3 (mg/g), cis-Nerolidol 0.27 (mg/g), Camphene 0.03 (mg/g), Fenchol 0.24 (mg/g), Borneol 0.03 (mg/g), alpha-Bisabolol 0.72 (mg/g), Caryophyllene oxide 0.21 (mg/g), Bisabolene 1.52 (mg/g), BetaCaryophyllene 4.33 (mg/g), beta-Farnesene 0.14 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Anti-ischemic, Appetite Stimulant, Neuroprotective, Anti-emetic, Anti-anxiety (Anxiolytic), Bronchodilator, Anti-spasmotic, Anti-psychotic.

### Formula #142

(a) one, two, three, 4, 5, 6, 7, 8, 9, 10 or all of the following terepenes: Linalool; Camphene; alpha-Pinene; beta-Pinene; Fenchol; Terpineol; Myrcene; Borneol; D-Limonene; Bisabolene; BetaCaryophyllene; beta-Farnesene; Humulene; Nerolidol; cis-Nerolidol; trans-beta-Ocimene;

Optionally formulated as: Linalool 2.02 (mg/g), Camphene 0.06 (mg/g), alpha-Pinene 0.29 (mg/g), beta-Pinene 0.91 (mg/g), Fenchol 0.3 (mg/g), Terpineol 0.29 (mg/g), Myrcene 1.86 (mg/g), Borneol 0.03 (mg/g), D-Limonene 2.41 (mg/g), Bisabolene 1.07 (mg/g), BetaCaryophyllene 11.33 (mg/g), beta-Farnesene 1.14 (mg/g), Humulene 0.84 (mg/g), Nerolidol 1.09 (mg/g), cis-Nerolidol 0.17 (mg/g), trans-beta-Ocimene 0.1 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-spasmotic, Immunosuppressive, Anti-psychotic, Appetite Stimulant, Anti-ischemic, Bronchodilator, Neuroprotective, Anti-anxiety (Anxiolytic).

### Formula #143

(a) one, two, three, 4, 5, 6, 7, 8, 9, 10 or all of the following terepenes: Linalool; Camphene; alpha-Pinene; Fenchone; beta-Pinene; Fenchol; Terpineol; Myrcene; (3-Eudesmol; Guaiol; D-Limonene; alpha-Bisabolol; Caryophyllene oxide; BetaCaryophyllene; Humulene;

Optionally formulated as: Linalool 0.9 (mg/g), Camphene 0.05 (mg/g), alpha-Pinene 0.22 (mg/g), Fenchone 0.06 (mg/g), beta-Pinene 0.68 (mg/g), Fenchol 0.18 (mg/g), Terpineol 0.15 (mg/g), Myrcene 0.9 (mg/g), β-Eudesmol 0.44 (mg/g), Guaiol 2.46 (mg/g), D-Limonene 1.04 (mg/g), alpha-Bisabolol 2.74 (mg/g), Caryophyllene oxide 0.4 (mg/g), BetaCaryophyllene 4.4 (mg/g), Humulene 0.38 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Anti-anxiety (Anxiolytic), Anti-emetic, Bronchodilator, Anti-ischemic, Anti-spasmotic, Appetite Stimulant, Neuroprotective, Immunosuppressive.

### Formula #144

(a) one, two, three, 4, 5, 6, 7, 8, 9, 10 or all of the following terepenes: Linalool; alpha-Pinene; beta-Pinene; Myrcene; D-Limonene; trans-beta-Ocimene; BetaCaryophyllene; beta-Farnesene; Humulene;

Optionally formulated as: Linalool 0.19 (mg/g), alpha-Pinene 0.51 (mg/g), beta-Pinene 0.34 (mg/g), Myrcene 2.3 (mg/g), D-Limonene 0.19 (mg/g), trans-beta-Ocimene 0.14 (mg/g), BetaCaryophyllene 1.7 (mg/g), beta-Farnesene 0.17 (mg/g), Humulene 0.16 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-ischemic, Appetite Stimulant, Anti-anxiety (Anxiolytic), Neuroprotective, Immunosuppressive, Anti-spasmotic, Anti-psychotic, Bronchodilator.

### Formula #145

(a) one, two, three, 4, 5, 6, 7, 8, 9, 10 or all of the following terepenes: Linalool; Camphene; alpha-Pinene; beta-Pinene; Fenchol; Terpineol; Myrcene; Borneol; D-Limonene; alpha-Bisabolol; Caryophyllene oxide; Bisabolene; BetaCaryophyllene; beta-Farnesene; Humulene; cis-Nerolidol;

Optionally formulated as: Linalool 1.08 (mg/g), Camphene 0.05 (mg/g), alpha-Pinene 0.15 (mg/g), beta-Pinene 0.46 (mg/g), Fenchol 0.18 (mg/g), Terpineol 0.16 (mg/g), Myrcene 2.13 (mg/g), Borneol 0.04 (mg/g), D-Limonene 1.47 (mg/g), alpha-Bisabolol 0.76 (mg/g), Caryophyllene oxide 0.1 (mg/g), Bisabolene 0.82 (mg/g), BetaCaryophyllene 7.55 (mg/g), beta-Farnesene 0.09 (mg/g), Humulene 0.56 (mg/g), cis-Nerolidol 0.11 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Anti-emetic, Anti-ischemic, Neuroprotective, Anti-spasmotic, Anti-anxiety (Anxiolytic), Bronchodilator, Appetite Stimulant, Immunosuppressive

### Formula #146

(a) one, two, three, 4, 5, 6, 7, 8, 9, 10 or all of the following terepenes: Linalool; alpha-Pinene; beta-Pinene; Fenchol; Terpineol; Myrcene; β-Eudesmol; Guaiol; D-Limonene; Humulene; cis-Nerolidol; trans-beta-Ocimene; Borneol; alpha-Bisabolol; Bisabolene; BetaCaryophyllene;

Optionally formulated as: Linalool 2.04 (mg/g), alpha-Pinene 0.06 (mg/g), beta-Pinene 0.26 (mg/g), Fenchol 0.17 (mg/g), Terpineol 0.14 (mg/g), Myrcene 7.01 (mg/g), β-Eudesmol 0.77 (mg/g), Guaiol 3.7 (mg/g), D-Limonene 0.75 (mg/g), Humulene 0.22 (mg/g), cis-Nerolidol 0.16 (mg/g), trans-beta-Ocimene 0.21 (mg/g), Borneol 0.05 (mg/g), alpha-Bisabolol 5.28 (mg/g), Bisabolene 1.92 (mg/g), BetaCaryophyllene 2.68 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-spasmotic, Anti-anxiety (Anxiolytic), Anti-ischemic, Appetite Stimulant, Bronchodilator, Anti-emetic, Anti-psychotic, Neuroprotective, Immunosuppressive.

### Formula #147

(a) one, two, three, 4, 5, 6, 7, 8, 9, 10 or all of the following terepenes: Linalool; alpha-Pinene; beta-Pinene; Fenchol; Terpineol; Myrcene; Borneol; D-Limonene; alpha-Bisabolol; BetaCaryophyllene; alpha-Farnesene; Humulene; Nerolidol; cis-Nerolidol; Bisabolene;

Optionally formulated as: Linalool 0.87 (mg/g), alpha-Pinene 0.1 (mg/g), beta-Pinene 0.38 (mg/g), Fenchol 0.33 (mg/g), Terpineol 0.29 (mg/g), Myrcene 0.71 (mg/g), Borneol 0.05 (mg/g), D-Limonene 1.57 (mg/g), alpha-Bisabolol 1.98 (mg/g), BetaCaryophyllene 7.75 (mg/g), alpha-Farnesene 2.65 (mg/g), Humulene 0.62 (mg/g), Nerolidol 0.64 (mg/g), cis-Nerolidol 0.29 (mg/g), Bisabolene 2.84 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Anti-emetic, Immunosuppressive, Appetite Stimulant, Neuroprotective, Anti-anxiety (Anxiolytic), Anti-spasmotic, Bronchodilator, Anti-psychotic.

### Formula #148

(a) one, two, three, 4, 5, 6, 7, 8, 9, 10 or all of the following terepenes: Linalool; alpha-Pinene; beta-Pinene; Terpineol; Myrcene; D-Limonene; Humulene; Nerolidol; Camphene; Fenchol; Borneol; β-Eudesmol; Guaiol; BetaCaryophyllene;

Optionally formulated as: Linalool 1 (mg/g), alpha-Pinene 0.23 (mg/g), beta-Pinene 0.85 (mg/g), Terpineol 0.32 (mg/g), Myrcene 6.96 (mg/g), D-Limonene 2.52 (mg/g), Humulene 0.09 (mg/g), Nerolidol 0.7 (mg/g), Camphene 0.09 (mg/g), Fenchol 0.32 (mg/g), Borneol 0.06 (mg/g), β-Eudesmol 0.43 (mg/g), Guaiol 2.37 (mg/g), BetaCaryophyllene 0.82 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Appetite Stimulant, Neuroprotective, Anti-spasmotic, Bronchodilator, Anti-ischemic, Immunosuppressive, Anti-anxiety (Anxiolytic), Anti-psychotic.

### Formula #149

(a) one, two, three, 4, 5, 6, 7, 8, 9, 10 or all of the following terepenes: Linalool; alpha-Pinene; alpha-Phellandrene; gamma-Terpinene; alpha-Terpinene; 4-Carvomenthenol; Terpinolene; beta-Pinene; Fenchol; Terpineol; Sabinene; 3-Carene; Myrcene; D-Menthol; D-Limonene; alpha-Bisabolol; Bisabolene; BetaCaryophyllene; Humulene; Nerolidol; cis-Nerolidol;

Optionally formulated as: Linalool 1.59 (mg/g), alpha-Pinene 0.22 (mg/g), alpha-Phellandrene 0.18 (mg/g), gamma-Terpinene 0.05 (mg/g), alpha-Terpinene 0.09 (mg/g), 4-Carvomenthenol 0.07 (mg/g), Terpinolene 2.05 (mg/g), beta-Pinene 0.7 (mg/g), Fenchol 0.09 (mg/g), Terpineol 0.23 (mg/g), Sabinene 0.05 (mg/g), 3-Carene 0.13 (mg/g), Myrcene 3.05 (mg/g), D-Menthol 0.09 (mg/g), D-Limonene 0.59 (mg/g), alpha-Bisabolol 1.96 (mg/g), Bisabolene 0.59 (mg/g), BetaCaryophyllene 5.92 (mg/g), Humulene 0.49 (mg/g), Nerolidol 1.41 (mg/g), cis-Nerolidol 0.27 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-anxiety (Anxiolytic), Immunosuppressive, Appetite Stimulant, Anti-emetic, Anti-ischemic, Neuroprotective, Bronchodilator, Anti-psychotic, Anti-spasmotic

### Formula #150

(a) one, two, three, 4, 5, 6, 7, 8, 9, 10 or all of the following terepenes: Terpinolene; Terpineol; Eucalyptol; Linalool; Camphene; alpha-Pinene; alpha-Phellandrene; gamma-Terpinene; alpha-Terpinene; 4-Carvomenthenol; beta-Pinene; Fenchol; Sabinene; 3-Carene; Myrcene; Borneol; D-Limonene; BetaCaryophyllene; beta-Farnesene; Humulene; Nerolidol; trans-beta-Ocimene;

Optionally formulated as: Terpinolene 2.24 (mg/g), Terpineol 0.32 (mg/g), Eucalyptol 0.08 (mg/g), Linalool 0.27 (mg/g), Camphene 0.06 (mg/g), alpha-Pinene 0.51 (mg/g), alpha-Phellandrene 0.2 (mg/g), gamma-Terpinene 0.05 (mg/g), alpha-Terpinene 0.09 (mg/g), 4-Carvomenthenol 0.05 (mg/g), beta-Pinene 1.21 (mg/g), Fenchol 0.23 (mg/g), Sabinene 0.07 (mg/g), 3-Carene 0.14 (mg/g), Myrcene 1.28 (mg/g), Borneol 0.04 (mg/g), D-Limonene 1.33 (mg/g), BetaCaryophyllene 3.12 (mg/g), beta-Farnesene 0.31 (mg/g), Humulene 0.4 (mg/g), Nerolidol 0.82 (mg/g), trans-beta-Ocimene 0.71 (mg/g),

(b) in alternative embodiment, this formulation is used to treat, ameliorate, lesson the symptoms of, or prevent any one of the following conditions, or be used as an: Anti-bacterial, Anti-insomnia, Anti-depressant, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-spasmotic, Anti-emetic, Appetite Stimulant, Anti-ischemic, Anti-psychotic, Immunosuppressive, Anti-anxiety (Anxiolytic), Neuroprotective.

### Formula #151

(a) one, two, three, 4, 5, 6, 7, 8, 9, 10 or all of the following terepenes: beta-Pinene; trans-beta-Ocimene; Linalool; Myrcene; β-Eudesmol; D-Limonene; alpha-Bisabolol; Bisabolene; BetaCaryophyllene; Humulene;

Optionally formulated as: beta-Pinene 0.13 (mg/g), trans-beta-Ocimene 0.16 (mg/g), Linalool 0.78 (mg/g), Myrcene 3.09 (mg/g), β-Eudesmol 0.14 (mg/g), D-Limonene 0.29 (mg/g), alpha-Bisabolol 1.05 (mg/g), Bisabolene 0.87 (mg/g), BetaCaryophyllene 0.97 (mg/g), Humulene 0.1 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Analgesic, Anti-inflammatory, Anti-cancer, Appetite Suppressant,

(c) in alternative embodiment, this formulation is used to treat, ameliorate, lesson the symptoms of, or prevent any one of the following conditions, or be used as an: Appetite Stimulant, Bronchodilator, Neuroprotective, Anti-ischemic, Anti-emetic, Immunosuppressive, Anti-spasmotic, Anti-anxiety (Anxiolytic), Anti-psychotic.

### Formula #152

(a) one, two, three, 4, 5, 6, 7, 8, 9, 10 or all of the following terepenes: Linalool; alpha-Pinene; alpha-Phellandrene; gamma-Terpinene; alpha-Terpinene; Terpinolene; beta-Pinene; Fenchol; Terpineol; Sabinene; 3-Carene; Myrcene; β-Eudesmol; Guaiol; D-Limonene; BetaCaryophyllene; Humulene; trans-beta-Ocimene;

Optionally formulated as: Linalool 0.28 (mg/g), alpha-Pinene 0.33 (mg/g), alpha-Phellandrene 0.25 (mg/g), gamma-Terpinene 0.06 (mg/g), alpha-Terpinene 0.12 (mg/g), Terpinolene 2.9 (mg/g), beta-Pinene 1.03 (mg/g), Fenchol 0.07 (mg/g), Terpineol 0.17 (mg/g), Sabinene 0.06 (mg/g), 3-Carene 0.17 (mg/g), Myrcene 4.8 (mg/g), β-Eudesmol 0.18 (mg/g), Guaiol 1.21 (mg/g), D-Limonene 0.7 (mg/g), BetaCaryophyllene 0.45 (mg/g), Humulene 0.05 (mg/g), trans-beta-Ocimene 0.8 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Neuroprotective, Appetite Stimulant, Anti-ischemic, Bronchodilator, Anti-anxiety (Anxiolytic), Anti-emetic, Immunosuppressive, Anti-spasmotic.

### Formula #153

(a) one, two, three, 4, 5, 6, 7, 8, 9, 10 or all of the following terepenes: Linalool; Camphene; alpha-Pinene; Fenchone; beta-Pinene; Fenchol; Terpineol; Myrcene; Borneol; β-Eudesmol; Guaiol; D-Limonene; BetaCaryophyllene; beta-Farnesene; Humulene; trans-beta-Ocimene;

Optionally formulated as: Linalool 0.27 (mg/g), Camphene 0.08 (mg/g), alpha-Pinene 0.42 (mg/g), Fenchone 0.07 (mg/g), beta-Pinene 0.76 (mg/g), Fenchol 0.22 (mg/g), Terpineol 0.22 (mg/g), Myrcene 3.31 (mg/g), Borneol 0.04 (mg/g), β-Eudesmol 0.21 (mg/g), Guaiol 1.08 (mg/g), D-Limonene 2.6 (mg/g), BetaCaryophyllene 2.14 (mg/g), beta-Farnesene 0.2 (mg/g), Humulene 0.19 (mg/g), trans-beta-Ocimene 0.24 (mg/g), (b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory,

Analgesic, Anti-cancer, Appetite Suppressant, (c) in alternative embodiment, this formulation is used to treat, ameliorate, lesson the symptoms of, or prevent any one of the following conditions, or be used as an: Immunosuppressive, Anti-psychotic, Anti-ischemic, Neuroprotective, Anti-emetic, Anti-spasmotic, Appetite Stimulant, Anti-anxiety (Anxiolytic), Bronchodilator.

### Formula #154

(a) one, two, three, 4, 5, 6, 7, 8, 9, 10 or all of the following terepenes: Linalool; Camphene; alpha-Pinene; beta-Pinene; Fenchol; Terpineol; Myrcene; Borneol; D-Limonene; alpha-Bisabolol; Bisabolene; BetaCaryophyllene; alpha-Farnesene; beta-Farnesene; Humulene; cis-Nerolidol;

Optionally formulated as: Linalool 2.59 (mg/g), Camphene 0.06 (mg/g), alpha-Pinene 0.2 (mg/g), beta-Pinene 0.63 (mg/g), Fenchol 0.34 (mg/g), Terpineol 0.3 (mg/g), Myrcene 2.41 (mg/g), Borneol 0.05 (mg/g), D-Limonene 2.28 (mg/g), alpha-Bisabolol 0.8 (mg/g), Bisabolene 1.21 (mg/g), BetaCaryophyllene 3.29 (mg/g), alpha-Farnesene 1.24 (mg/g), beta-Farnesene 0.1 (mg/g), Humulene 0.31 (mg/g), cis-Nerolidol 0.16 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Immunosuppressive, Neuroprotective, Anti-spasmotic, Anti-anxiety (Anxiolytic), Anti-ischemic, Appetite Stimulant, Anti-emetic, Anti-psychotic, Bronchodilator.

### Formula #155

(a) one, two, three, 4, 5, 6, 7, 8, 9, 10 or all of the following terepenes: Linalool; Camphene; alpha-Pinene; beta-Pinene; Fenchol; Myrcene; Borneol; β-Eudesmol; Guaiol; D-Limonene; Bisabolene; BetaCaryophyllene; Humulene;

Optionally formulated as: Linalool 0.38 (mg/g), Camphene 0.04 (mg/g), alpha-Pinene 0.14 (mg/g), beta-Pinene 0.4 (mg/g), Fenchol 0.15 (mg/g), Myrcene 3.31 (mg/g), Borneol 0.03 (mg/g), β-Eudesmol 0.26 (mg/g), Guaiol 1.65 (mg/g), D-Limonene 1.3 (mg/g), Bisabolene 1 (mg/g), BetaCaryophyllene 2.14 (mg/g), Humulene 0.18 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-spasmotic, Anti-ischemic, Appetite Stimulant, Neuroprotective, Anti-anxiety (Anxiolytic), Bronchodilator, Anti-psychotic, Immunosuppressive

### Formula #156

(a) one, two, three, 4, 5, 6, 7, 8, 9, 10 or all of the following terepenes: Linalool; alpha-Pinene; alpha-Phellandrene; gamma-Terpinene; alpha-Terpinene; Terpinolene; beta-Pinene; Terpineol; Sabinene; Myrcene; D-Limonene; Humulene; Camphene; Fenchol; 3-Carene; Borneol; β-Eudesmol; Guaiol; alpha-Bisabolol; BetaCaryophyllene;

Optionally formulated as: Linalool 0.7 (mg/g), alpha-Pinene 0.31 (mg/g), alpha-Phellandrene 0.21 (mg/g), gamma-Terpinene 0.05 (mg/g), alpha-Terpinene 0.1 (mg/g), Terpinolene 2.72 (mg/g), beta-Pinene 0.86 (mg/g), Terpineol 0.25 (mg/g), Sabinene 0.08 (mg/g), Myrcene 3.75 (mg/g), D-Limonene 1.12 (mg/g), Humulene 0.14 (mg/g), Camphene 0.03 (mg/g), Fenchol 0.13 (mg/g), 3-Carene 0.13 (mg/g), Borneol 0.03 (mg/g), β-Eudesmol 0.21 (mg/g), Guaiol 1.12 (mg/g), alpha-Bisabolol 0.96 (mg/g), BetaCaryophyllene 1.29 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-emetic, Anti-anxiety (Anxiolytic), Anti-psychotic, Anti-spasmotic, Immunosuppressive, Bronchodilator, Neuroprotective, Anti-ischemic.

### Formula #157

(a) one, two, three, 4, 5, 6, 7, 8, 9, 10 or all of the following terepenes: Linalool; Camphene; alpha-Pinene; Terpinolene; beta-Pinene; Fenchol; Terpineol; Myrcene; D-Limonene; BetaCaryophyllene; alpha-Farnesene; Humulene; cis-Nerolidol; Valencene; Borneol; alpha-Bisabolol; Bisabolene;

Optionally formulated as: Linalool 0.83 (mg/g), Camphene 0.07 (mg/g), alpha-Pinene 0.21 (mg/g), Terpinolene 0.03 (mg/g), beta-Pinene 0.62 (mg/g), Fenchol 0.36 (mg/g), Terpineol 0.29 (mg/g), Myrcene 3.36 (mg/g), D-Limonene 3.17 (mg/g), BetaCaryophyllene 7.32 (mg/g), alpha-Farnesene 2.38 (mg/g), Humulene 0.7 (mg/g), cis-Nerolidol 0.15 (mg/g), Valencene 0.49 (mg/g), Borneol 0.04 (mg/g), alpha-Bisabolol 3.3 (mg/g), Bisabolene 2.65 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Appetite Stimulant, Anti-emetic, Anti-psychotic, Anti-ischemic, Neuroprotective, Anti-anxiety (Anxiolytic), Immunosuppressive, Anti-spasmotic.

### Formula #158

(a) one, two, three, 4, 5, 6, 7, 8, 9, 10 or all of the following terepenes: alpha-Pinene; beta-Pinene; Terpineol; Linalool; Camphene; Fenchol; Myrcene; Borneol; (3-Eudesmol; Guaiol; D-Limonene; alpha-Bisabolol; BetaCaryophyllene; alpha-Farnesene; Humulene; cis-Nerolidol;

Optionally formulated as: alpha-Pinene 0.28 (mg/g), beta-Pinene 0.75 (mg/g), Terpineol 0.23 (mg/g), Linalool 1.56 (mg/g), Camphene 0.09 (mg/g), Fenchol 0.3 (mg/g), Myrcene 0.68 (mg/g), Borneol 0.05 (mg/g), β-Eudesmol 0.35 (mg/g), Guaiol 2 (mg/g), D-Limonene 3.65 (mg/g), alpha-Bisabolol 0.89 (mg/g), BetaCaryophyllene 1.42 (mg/g), alpha-Farnesene 1.17 (mg/g), Humulene 0.17 (mg/g), cis-Nerolidol 0.04 (mg/g),

(b) in alternative embodiment, this formulation is used to treat, ameliorate, lesson the symptoms of, or prevent any one of the following conditions, or be used as an: Anti-inflammatory, Anti-bacterial, Anti-depressant, Anti-insomnia, Anti-convulsive, Analgesic, Anti-cancer, Appetite Suppressant,

(c) in alternative embodiment, this formulation is used to treat, ameliorate, lesson the symptoms of, or prevent any one of the following conditions, or be used as an: Appetite Stimulant, Anti-ischemic, Anti-emetic, Anti-anxiety (Anxiolytic), Anti-spasmotic, Bronchodilator, Neuroprotective, Anti-psychotic, Immunosuppressive.

### Formula #159

(a) one, two, three, 4, 5, 6, 7, 8, 9, 10 or all of the following terepenes: Linalool; Camphene; alpha-Pinene; beta-Pinene; Fenchol; Terpineol; Myrcene; Borneol; D-Limonene; BetaCaryophyllene; beta-Farnesene; Humulene; Nerolidol; cis-Nerolidol;

Optionally formulated as: Linalool 1.57 (mg/g), Camphene 0.06 (mg/g), alpha-Pinene 0.21 (mg/g), beta-Pinene 0.48 (mg/g), Fenchol 0.18 (mg/g), Terpineol 0.11 (mg/g), Myrcene 1.33 (mg/g), Borneol 0.04 (mg/g), D-Limonene 2.37 (mg/g), BetaCaryophyllene 1.03 (mg/g), beta-Farnesene 0.13 (mg/g), Humulene 0.14 (mg/g), Nerolidol 0.96 (mg/g), cis-Nerolidol 0.12 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Immunosuppressive, Anti-spasmotic, Anti-ischemic, Neuroprotective, Bronchodilator, Anti-anxiety (Anxiolytic), Anti-emetic, Appetite Stimulant

### Formula #160

(a) one, two, three, 4, 5, 6, 7, 8, 9, 10 or all of the following terepenes: Linalool; Camphene; alpha-Pinene; beta-Pinene; Fenchol; Terpineol; Myrcene; Borneol; D-Limonene; alpha-Bisabolol; Caryophyllene oxide; BetaCaryophyllene; alpha-Farnesene; Humulene; cis-Nerolidol; Bisabolene;

Optionally formulated as: Linalool 3 (mg/g), Camphene 0.06 (mg/g), alpha-Pinene 0.18 (mg/g), beta-Pinene 0.49 (mg/g), Fenchol 0.3 (mg/g), Terpineol 0.24 (mg/g), Myrcene 1.87 (mg/g), Borneol 0.04 (mg/g), D-Limonene 2.12 (mg/g), alpha-Bisabolol 2.96 (mg/g), Caryophyllene oxide 0.09 (mg/g), BetaCaryophyllene 9.67 (mg/g), alpha-Farnesene 2.57 (mg/g), Humulene 1.25 (mg/g), cis-Nerolidol 0.24 (mg/g), Bisabolene 2.45 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-psychotic, Bronchodilator, Anti-emetic, Neuroprotective, Appetite Stimulant, Anti-anxiety (Anxiolytic), Anti-spasmotic, Immunosuppressive, Anti-ischemic

### Formula #161

(a) one, two, three, 4, 5, 6, 7, 8, 9, 10 or all of the following terepenes: Linalool; Camphene; alpha-Pinene; beta-Pinene; Fenchol; Myrcene; Borneol; D-Limonene; alpha-Bisabolol; Caryophyllene oxide; Bisabolene; BetaCaryophyllene; alpha-Farnesene; Humulene; Nerolidol; cis-Nerolidol; trans-beta-Ocimene;

Optionally formulated as: Linalool 0.79 (mg/g), Camphene 0.04 (mg/g), alpha-Pinene 0.43 (mg/g), beta-Pinene 0.34 (mg/g), Fenchol 0.16 (mg/g), Myrcene 2.01 (mg/g), Borneol 0.03 (mg/g), D-Limonene 0.47 (mg/g), alpha-Bisabolol 1.9 (mg/g), Caryophyllene oxide 0.07 (mg/g), Bisabolene 0.64 (mg/g), BetaCaryophyllene 1.56 (mg/g), alpha-Farnesene 0.9 (mg/g), Humulene 0.22 (mg/g), Nerolidol 0.67 (mg/g), cis-Nerolidol 0.12 (mg/g), trans-beta-Ocimene 0.47 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Anti-psychotic, Neuroprotective, Anti-spasmotic, Anti-ischemic, Anti-anxiety (Anxiolytic), Bronchodilator, Immunosuppressive, Appetite Stimulant

### Formula #162

(a) one, two, three, 4, 5, 6, 7, 8, 9, 10 or all of the following terepenes: Linalool; Camphene; alpha-Pinene; beta-Pinene; Fenchol; Terpineol; Myrcene; Borneol; D-Limonene; alpha-Bisabolol; Bisabolene; BetaCaryophyllene; Humulene; cis-Nerolidol; trans-beta-Ocimene;

Optionally formulated as: Linalool 0.18 (mg/g), Camphene 0.04 (mg/g), alpha-Pinene 0.99 (mg/g), beta-Pinene 0.53 (mg/g), Fenchol 0.15 (mg/g), Terpineol 0.1 (mg/g), Myrcene 1.68 (mg/g), Borneol 0.03 (mg/g), D-Limonene 0.71 (mg/g), alpha-Bisabolol 0.86 (mg/g), Bisabolene 0.77 (mg/g), BetaCaryophyllene 0.88 (mg/g), Humulene 0.1 (mg/g), cis-Nerolidol 0.05 (mg/g), trans-beta-Ocimene 0.09 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-ischemic, Appetite Stimulant, Immunosuppressive, Anti-psychotic, Neuroprotective, Anti-emetic, Anti-spasmotic, Anti-anxiety (Anxiolytic), Bronchodilator

### Formula #163

(a) one, two, three, 4, 5, 6, 7, 8, 9, 10 or all of the following terepenes: beta-Pinene; Linalool; alpha-Pinene; Fenchol; Myrcene; Borneol; D-Limonene; alpha-Bisabolol; Caryophyllene oxide; Bisabolene; BetaCaryophyllene; beta-Farnesene; Humulene; cis-Nerolidol;

Optionally formulated as: beta-Pinene 0.25 (mg/g), Linalool 0.79 (mg/g), alpha-Pinene 0.1 (mg/g), Fenchol 0.17 (mg/g), Myrcene 2.18 (mg/g), Borneol 0.03 (mg/g), D-Limonene 0.76 (mg/g), alpha-Bisabolol 2.34 (mg/g), Caryophyllene oxide 0.12 (mg/g), Bisabolene 0.67 (mg/g), BetaCaryophyllene 5.71 (mg/g), beta-Farnesene 0.35 (mg/g), Humulene 0.7 (mg/g), cis-Nerolidol 0.04 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Bronchodilator, Anti-psychotic, Anti-emetic, Neuroprotective, Anti-anxiety (Anxiolytic), Appetite Stimulant, Anti-ischemic, Immunosuppressive, Anti-spasmotic.

### Formula #164

(a) one, two, three, 4, 5, 6, 7, 8, 9, 10 or all of the following terepenes: Linalool; alpha-Pinene; Terpinolene; beta-Pinene; Terpineol; Myrcene; D-Limonene; Nerolidol; Valencene; Camphene; Fenchone; Fenchol; Borneol; alpha-Bisabolol; Caryophyllene oxide; Bisabolene; BetaCaryophyllene; alpha-Farnesene; beta-Farnesene; Humulene;

Optionally formulated as: Linalool 1.08 (mg/g), alpha-Pinene 0.47 (mg/g), Terpinolene 0.06 (mg/g), beta-Pinene 0.67 (mg/g), Terpineol 0.36 (mg/g), Myrcene 3.3 (mg/g), D-Limonene 3.5 (mg/g), Nerolidol 1.24 (mg/g), Valencene 0.2 (mg/g), Camphene 0.14 (mg/g), Fenchone 0.03 (mg/g), Fenchol 0.59 (mg/g), Borneol 0.12 (mg/g), alpha-Bisabolol 0.85 (mg/g), Caryophyllene oxide 0.12 (mg/g), Bisabolene 0.4 (mg/g), BetaCaryophyllene 5.8 (mg/g), alpha-Farnesene 0.35 (mg/g), beta-Farnesene 0.06 (mg/g), Humulene 1.44 (mg/g),

(b) in alternative embodiment, this formulation is used to treat, ameliorate, lesson the symptoms of, or prevent any one of the following conditions, or be used as an: Anti-bacterial, Anti-depressant, Anti-insomnia, Anti-convulsive, Anti-inflammatory, Analgesic, Anti-cancer, Appetite Suppressant,

(c) in alternative embodiment, this formulation is used to treat, ameliorate, lesson the symptoms of, or prevent any one of the following conditions, or be used as an: Anti-emetic, Appetite Stimulant, Anti-ischemic, Anti-spasmotic, Neuroprotective, Bronchodilator, Anti-psychotic, Immunosuppressive, Anti-anxiety (Anxiolytic).

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Isolation and Chemical Characterization of Compounds from Inflorescence of Cannabis Sativa L

This example describes exemplary methods for the isolation and chemical characterization of essential oils, phytochemicals and pharmaceutically active components from the inflorescence of specific strains of Cannabis Sativa L. and the reformulation of these chemical profiles, and making a synthetic, non-natural formulation comprising a selected mix of these plant-derived chemicals.

The complete terpene and/or terpenoid components from biomass of each of these strains are quantitatively extracted using several techniques including freeze-dried extraction, super-critical CO₂ extraction, and vacuum-oven extraction with optimized cold-trap and short-path distillation. These extracted materials are analyzed by gas chromatography-mass spectrometry with single ion monitoring for over 50 different terpenes for which standards are available. This analytical data is used to create specific terpene/terpenoid profiles for each of the strains.

After the terpenes have been quantitatively extracted from the biomass of specific strains, the extracted materials are analyzed by gas chromatography-mass spectrometry (GCMS). Selected ion monitoring (SIM) allows detection limits in the range of 0.05% depending on the specific terpene.

Currently standards are available for 50 terpenes, and quantitative results are limited to those compounds, although other components are observed in the analysis and may be identified qualitatively by comparison of their mass spectra to the NIST mass spectral library.

Data from the GCMS analyses are tabulated to create recipes from which the terpene profiles may be reformulated using isolated plant-derived terpenes from other sources.

Standards are currently available for 19 cannabinoids and the distribution of these can be measured using high performance liquid chromatography (HPLC).

Different strains of Cannabis Sativa L. with validated genetics have been grown from seed under controlled consistent environmental conditions. The complete terpene components of these strains have been quantitatively extracted and analyzed by gas chromatography-mass spectrometry and the analytical data used to create specific terpene profiles for each of the strains. Recipes derived from these profiles are then used to re-create the specific combinations using isolated terpenes from other sources.

In alternative embodiments, reconstituted/synthetic formulations comprising phytochemical can replicate the flavor, odor, therapeutic effect and other characteristics of specific plant strains by reformulating the chemical profiles using combinations of isolated constituent compounds.

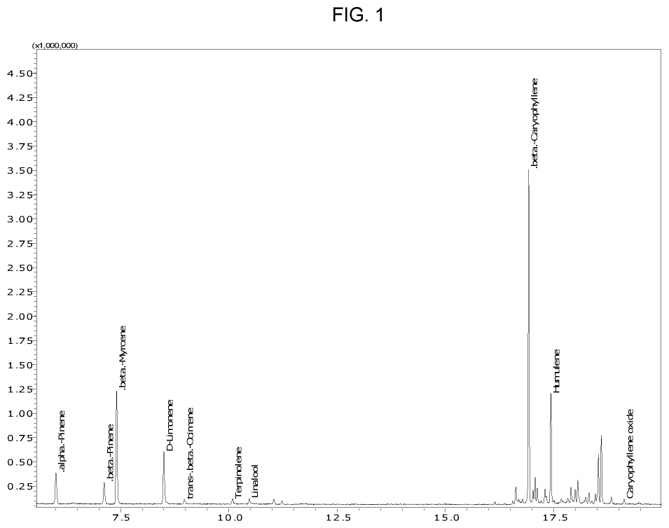

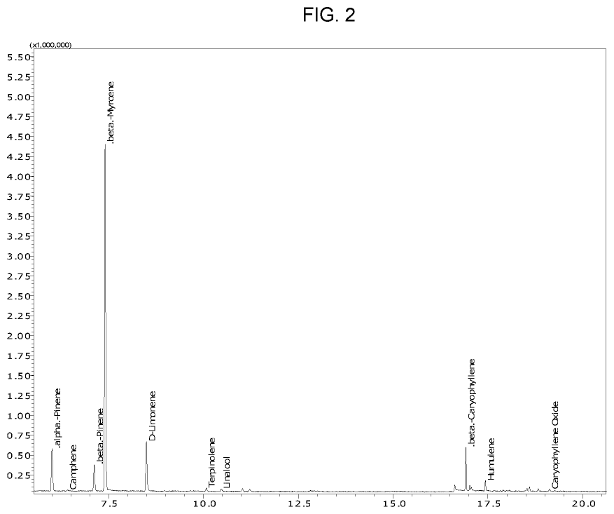

FIGS. 1 through 3 are GCMS chromatograms of the terpene extracts from three different strains of Cannabis Sativa L. with the most abundant terpenes identified. Table 1 shows the concentrations derived from this chromatographic data for 49 terpenes measured for each of the three strains.

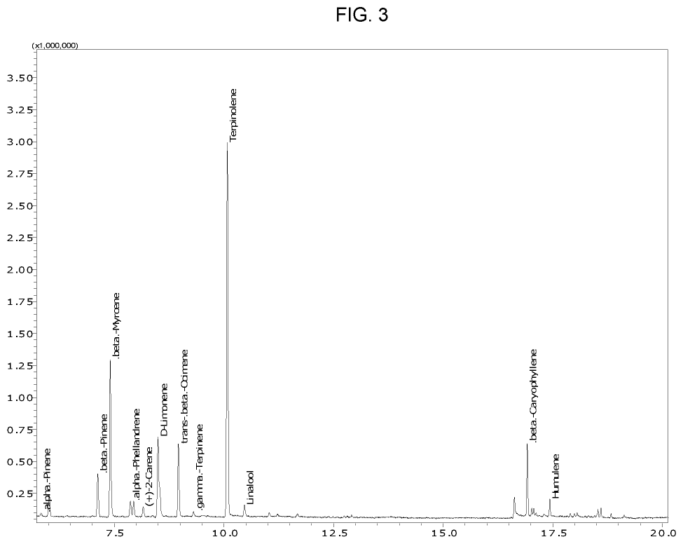

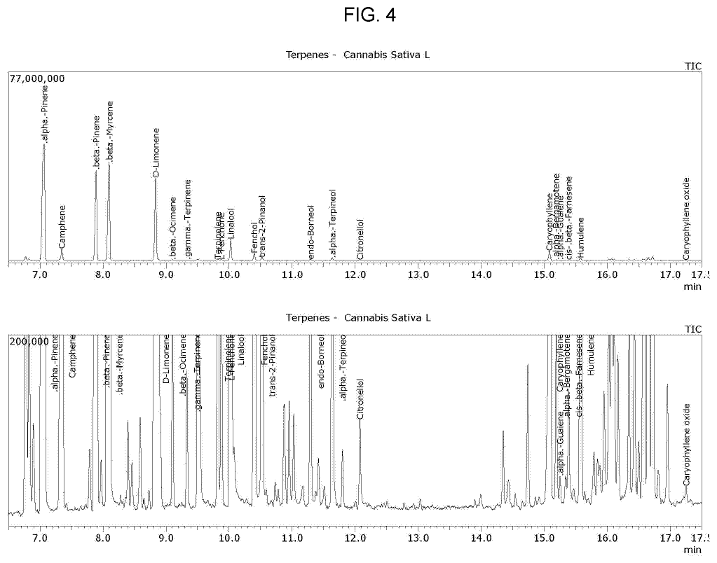

FIG. 4 shows the abundance of additional terpenes typically present in Cannabis Sativa L. The upper plot (graph) is scaled to the most abundant compounds; the lower plot (graph) is the same chromatogram expanded to show the many smaller peaks.

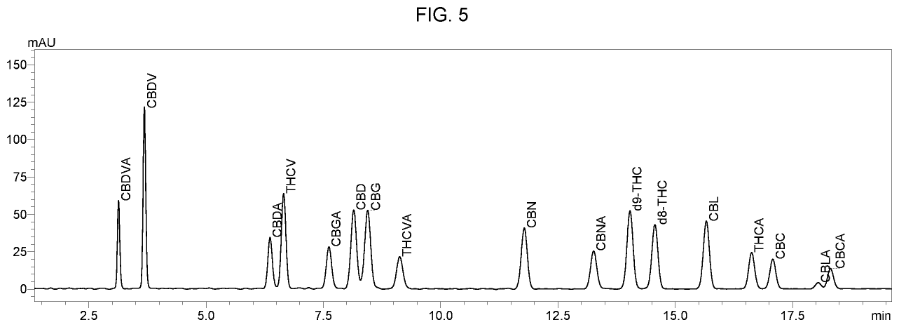

FIG. 5 shows the HPLC chromatograms of 17 cannabinoids.

TABLE 1
Summaries of 3 strain specific terpene profiles

| Terpene | Strain #1 | Strain #2 | Strain #3 |
|---|---|---|---|
| alpha-Pinene | 6.54% | 10.29% | 2.85% |
| Camphene | 0.21% | 0.22% | 0.19% |
| Sabinene | | | 0.10% |
| beta-Pinene | 3.70% | 5.01% | 4.44% |
| beta-Myrcene | 18.77% | 64.86% | 16.57% |
| alpha-Phellandrene | 0.06% | | 2.34% |
| Carene | 0.05% | | 1.81% |
| 1,4-Cineole | | | |
| alpha-Terpinene | 0.03% | | 1.11% |
| p-Cymene | | | 0.22% |
| Limonene | 7.63% | 8.93% | 7.13% |
| Eucalyptol | 0.20% | | 0.25% |
| alpha-Ocimene | 0.07% | | 0.12% |
| trans-beta-Ocimene | 0.90% | 0.10% | 7.83% |
| gamma-Terpinene | 0.02% | 0.01% | 0.47% |
| Sabinene hydrate | 0.03% | | 0.07% |
| Terpinolene | 1.08% | 0.87% | 44.56% |
| Fenchone | 0.07% | 0.04% | 0.08% |
| Linalool | 1.15% | 0.65% | 1.38% |
| Fenchol | 0.89% | 0.65% | 0.45% |
| Camphor | | | |
| Isopulegol | | | |
| Isoborneol | | | |
| Borneol | 0.06% | 0.03% | 0.03% |
| Menthol | | | |
| Terpinen-4-ol | | | 0.13% |
| Terpineol | 0.23% | 0.19% | 0.35% |
| Nerol | | | |
| Citronellol | | | |
| Pulegone | | | |
| L-Carvone | | | |
| Geraniol | | | |
| Geranyl acetate | | | |
| beta-Elemene | | | |
| alpha-Cedrene | | | |
| Caryophyllene | 38.90% | 6.09% | 5.50% |
| Aromadendrene | | | |
| trans-beta- | | | |
| Farnesene | 4.69% | 0.20% | 0.39% |
| Humulene | 11.82% | 1.29% | 1.18% |
| Valencene | | | |
| alpha-Farnesene | 2.10% | 0.15% | 0.25% |
| cis-Nerolidol | | | |
| trans-Nerolidol | | | |
| Caryophyllene | | | |
| oxide | 0.68% | 0.38% | 0.15% |
| Guaiol | 0.05% | 0.02% | 0.01% |
| Cedrol | 0.05% | 0.01% | 0.01% |
| alpha-Bisabolol | | | |
| Phytol 1 | | | |
| Phytol 2 | | | |

### Example 2: Quantitative Analysis of Terpenoid Isolates from Cannabis Strains by Gas Chromatography -Selected Ion Monitoring Mass Spectrometry

This example describes exemplary methods for the isolation and chemical characterization of essential oils, phytochemicals and pharmaceutically active components for use in synthetic formulations as provided herein.

For each sample between 85-115 mg of terpene isolate was weighed into a 15 mL polypropylene tube and dissolved in 10 mL High Performance Liquid Chromatography (HPLC) grade acetone containing 3,000 μg/mL cyclohexanone as an internal standard. Samples were vortexed and sonicated for 15 mins twice and then filtered using 0.2 micron polytetrafluoroethylene (PTFE) filters and diluted to a final dilution factor of 1:20 with 50 μL of filtered solution and 9504 of HPLC grade acetone into 2 mL glass autosampler vials. Prepared samples were then analyzed using a single quad GCMS. Chromatographic separation used helium as a carrier gas with a Rxi-5Sil MS™ (RESTEK) 30 m×0.25 mm×0.25 μm column. Scan and selected ion data were acquired. Analyte peaks were qualified by retention time, similarity of scanned spectra to standard spectra, and the ratios of SIM reference to target ions. Analytes were quantitated using SIM target ion areas compared with commercially available terpene standard mixes and standards prepared in-house.

### Example 3: Exemplary Protocol for Supercritical Carbon Dioxide Extraction of Terpenes

Preparation BioMass Materials
1. Grind the appropriate amount material (High-grade trim or flower only).
2. Record the appropriate data on the batch sheet.
3. Turn on the air compressor.
4. Turn on chiller/heater to 34 Deg F./130 Deg F.).
5. Load the extraction column with the prepared material
6. Place the gasket in the top hatch of the extraction vessel and bolt down.
7. Check that the CO₂ tanks are 900 psi or above for startup.

Operation Supercritical CO2 Extractor
1. Press process check and set the extraction high limit for THC to 1800 psi.
2. Set the THC separator target pressure to 800 psi.
3. Set the liquid accumulator pre-charge pressure to 800 psi, and the discharge pressure to 700 psi.
4. Set the extraction vessel discharge pressure to 1300 psi.
5. Set main separator starting pressure to 700 psi, and main separator target pressure to 400 psi.
6. Press hwv-1, then press temperature set point and enter 90. Press the enter arrow.
7. Press hwv-2, then press temperature set point and enter 121. Press the enter arrow.
8. Press run-time hours and enter the amount of hours.
9. Reset time (if time has elapsed).
10. Press THC process.
11. Go through the checklist:
Material loaded (if loaded and bolted down with the gasket check box)
Run time set
Separator checked (manual valve closed)
Backup separator checked (manual valve closed)
Extractor checked (visually inspect all bolts and put hands on them)
Heater/chiller checked(129 Deg F. and 40 Degree F.)
C02 tanks checked; 900 psi back 2 tanks, front tank at least 600 psi
Valve 8 closed; check that the valve behind the computer screen is closed
System air checked and drain the bottom of the air compressor
12. Press overview.
13. Press makeup pre-charge (accumulator will fill up to 800 psi).
14. Press checklist and make sure everything is checked.
15. Press system auto.
16. Press overview.
17. Press system start (will start pumping up the system).
18. Watch that the C02 tanks don't go below 600 psi. If so stop the system.
19. Press storage tank and open.
20. Press makeup gas open (will passively even out C02 tanks from the front tank).
21. Press system start (system will climb up to 1200 psi, then will say system circulating).
22. You can adjust both water temps to the actual temp to reach critical mass faster (adjust temps back to correct settings after reaching critical mass).
23. The system will stay circulating and will pull gas from the front C02 tank about every 5 min until the extraction has reached set point.
24. Once system reaches 1700 psi, it will begin to cycle (Screen will read “reached critical mass” in top left corner of display) Machine will run for alotted set time
25. At end of run, CO2 on its own; At which point it is necessary to manually recover enough CO2 to get storage tanks up to +900 psi

Recovery Process
1. Open storage tank.
2. Open V1.
3. Open V3, put gas pump in to position 3.
4. Open V5.
5. Press gas pump and press start on gas pump 2.
6. Once storage tanks reach +900 psi, press STOP SYSTEM.
7. Press system reset, then press manual.
8. Press overview.
9. Press hwv1 open and press hwv2 open.
10. At this point, SLOWLY feather handle on bottom of main separator to open valve and start draining raw extract into PYREX bowl for collection.
11. Open valve 8 slowly and watch the extractor psi drop.
12. Once drained, open valve 8 all the way and close hwv2 (DO NOT open the extractor top if HWV2 is open).
13. Press system stop (this will ensure everything on the screen is off and closed).
14. Check the gauge on top of the extraction lid to verify Opsi and begin opening the lid.
15. Take the material sample and vacuum out main extraction vessel.
16. Drain ALL condensate from the Fiac air-compressor located outside by manually opening the valve located at bottom left corner of compressor; Also drain any oil from filter regulator attached to left side of compressor by depressing the valve on bottom of apparatus.
17. At this point the system is completely shut-down, and you may proceed to processing recently extracted raw material and start preparations for next run if necessary.

### Rotovap Processing

### Pre-Run Checks

1. Check the blue drain valves and black vent caps on the receiving flasks to be sure they are closed (do not over-tighten). Also be sure the shut-off taps on top of the receiving flasks are in the proper position.
2. Check the inlet valve (trigger-like, located between boiling flask and expansion vessel) to be sure that it is in the fully-closed position, as well as any other connections from front to back to confirm tightness.
3. Be sure that the water bath is filled (water touching feet of manual flask handler); use only distilled water as the heating medium to avoid any hard water deposit on the bath.

### Dehydration

This process removes excess moisture from the raw extract in preparation for emulsification.
1. Turn ROTOVAPOR™ and recirculating chiller ON using green “I/O” toggle switch. (Vacuum pump stays switched ON and is controlled by chiller.) Using the green “TEMP. UP/DOWN” arrows on the bottom control panel, set the bath temperature at 55 degrees Celsius. Use the green “HEATER ON/OFF” button to activate the thermostat.
2. Prep the batch of raw extract (preferably fresh from the extractor) that is being dehydrated by mixing it well before transferring it to the evaporating flask. Properly label.
3. Raise the water bath using the “Lift>UP” button on upper control panel to immerse the flask enough that it displaces the bath water line to just below the overflow drain hole.
4. Turn on the rotation using the green “ROTATION ON/OFF” button on the upper control panel, and using the bottom dial adjust the speed of rotation to sufficiently melt extract (10-35 RPM).
5. On the top control panel press the “SET” button. Use the “UP/DOWN” arrows to select pressure set point. Use the top dial to adjust the vacuum to 60 mbar. Press “OK”. Chiller set point should be selected.
6. Again, use the dial to the right to adjust the chiller to 7 degrees Celsius. Press “OK”. (Press the green “START” button on the top control panel to start cooling the recirculating chiller only when your extract is sufficiently melted and ready for vacuum.)
7. Confirm that both the chiller and water bath are at set point, with extract evenly melted. Using the bottom dial, increase rotation to evenly disperse extract (75-100 RPM).
8. On the top control panel, press the green “START” button. Vacuum pump should start, and water removal begins. Once vacuum reaches set point, adjust rotation to keep extract agitated (45-65 RPM).
9. Record start time. Aerate every few minutes during the first 15-20 min of run-time.
10. After 45 min elapsed run-time vacuum should be increased to 55 mbar, and rotation should be increased to evenly disperse extract (75-100 RPM). Do a quick aeration of the system to help pull water.
11. After approximately 10-15 min decrease rotation (45-65 RPM) to agitate extract again. Increase bath temperature to 57 degrees Celsius. Do a quick aeration of the system to help pull water.
12. After another 30-45 min elapsed run-time slow rotation to 5 RPM and press the green “ROTATION ON/OFF” button to stop rotation altogether. Press the red “STOP” button on the top control panel twice to purge vacuum. (Chiller will continue to run automatically for 5 min; work swiftly to avoid a system shutdown)
13. Increase the bath temperature to 60 degrees Celsius. Lower the water bath completely using the “Lift>DOWN” button on lower control panel. Use the proper technique to safely remove the flask, letting it rest in the manual flask handler sitting on the bath.
14. Using a clean cloth dampened with ethyl alcohol carefully wipe away any excess water around the neck of the flask, then use a sterile pipette to remove any water droplets that are trapped within the extract. (Though unlikely, if the batch happens to be finished at this point skip to step #21; otherwise, proceed normally to step #15.)
15. Safely replace the evaporating flask using the proper technique and start the rotation using the “ROTATION ON/OFF” button, and increase speed to evenly disperse extract (75-100 RPM). If the chiller is still operating, press the green “START” button on the upper control panel to start the vacuum pump. Otherwise, start the chiller and allow it to reach the set temperature before starting the vacuum pump.
16. Once the pressure set-point is reached, decrease rotation (45-65 RPM) to once again agitate extract and increase vacuum pressure to 50 mbar. (Try not to exceed 20 min elapsed run-time at these finishing parameters)
17. Depending on water content a 400-600 g batch of raw extract should take about 2-2½ hours to completely de-water. (Note that there is hardly any visible moisture remaining inside the evaporating flask; extract should appear to be transparent)
18. Slow rotation to 5 RPM and press green “ROTATION ON/OFF” button to stop rotation altogether. Press the red “STOP” button on the top control panel three times to purge vacuum and stop chiller simultaneously if necessary.
19. Lower the water bath completely using the “Lift>DOWN” button on upper control panel. Use the proper technique to safely remove the flask, resting it on the manual flask handler.
20. Using a clean cloth dampened with ethyl alcohol carefully wipe away any excess water around the neck of the flask, then use a sterile pipette to remove any water droplets that are still trapped within the extract.
21. Record end time and weight in Operator Report. Extract is now ready for emulsification.
22. Ideally, after every dehydration a low-temperature sterilization (see page 5) will capture any essential oils remaining on the condensing coils, and minimize potential cross-contamination. Carefully drain the contents of the receiving flask into a graduated cylinder, label properly, and place in fridge for separation.

### Example 4: Exemplary Protocol for Freeze Drying Terpenes

Bio-Material Prep

Using grinder, reduce material if necessary; Only buds bigger than dime size should be ground.
1. Open lid; Fill with material to just below the rim
2. Close lid; secure
3. Hit JOG button while simultaneously rotating mixer handle located on lid
4. Reduce to desired size
5. Collect material now prepped for freeze dryer

Using the freeze dryer, remove the maximum amount of moisture possible
1. If the unit is dormant, connect the main power plug to the proper outlet on the wall to restore power.
2. The machine typically stays set at standard settings, revert to Manual located on top of machine to adjust if necessary.
3. Load your 10 lbs of material into the freeze dryer, spread evenly on shelves; Be sure the probe is touching the material being processed.
4. START ice bank; allow temperature to reach −40 F.
5. START vacuum; allow pressure to reach 1200MT.
6. Press F1 to start cycle; The machine will run for a duration of 16 hours before automatically shutting down, but machine can be stopped manually at any time.
7. To manually stop the cycle, press F2 upon completion.
8. Turn OFF vacuum.
9. Open the main valve located on the underside of unit, allowing pressure to release and equalize.
10. Empty shelves and collect the 10 lbs of dried material that is now ready

Essential Oil Collection
1. Turn OFF ice bank; turn ON defroster.
2. Open rear access door, and remove gasket.
3. Pause momentarily; remain present to allow defroster to increase chamber temp from −40 F to approx 37 F at which point liquid should become evident around the edges
4. Begin carefully removing ice from walls of chamber into collection vessel.
5. Cover vessel of collected ice with plastic wrap and label with proper strain identification.
6. Turn defroster OFF
7. Thoroughly clean chamber with lint-free cloth and ethanol to remove any leftover residue and/or debris in preparation for the next run.

A number of embodiments of the invention have been described. Nevertheless, it can be understood that various modifications may be made without departing from the spirit and scope of the invention. Accordingly, other embodiments are within the scope of the following claims.

1. A synthetic or non-natural formulation comprising a non-natural mix of bio-mass or plant-derived terpenes or terpenoids,
wherein:
(a) the terpenes or terpenoids comprise at least one, 2, 3, 4, 5, 6, 7, 8, 9, 10, 11, 12, 13, 14, 15, 16, 17, 18, 19 or 20 or more terpene or terpenoid species listed in Table 1, or at least one, 2, 3, 4, 5, 6, 7, 8, 9, 10, 11, 12, 13, 14, 15, 16, 17, 18, 19 or 20 or more of the following terpenes or terpenoids: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Famesene, (6E)-; beta-Ocimene; beta-Pinene; beta-Thujene; Bomeol; Camphene; Camphor; Cannabidiol; Cannabigerobc Acid; Carvone, (−)-; Caryophyllene Oxide; Cedrol; cis-2-Pinanol; cis-beta-ocimene; cis-Nerobdol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isobomeol, (−)-; Isopulegol; Limonene; Linalool; Menthol; Myrcene; Nerol; Nerobdol; p-Cymene; Phytol; Pulegone; Sabinene; Sabinene Hydrate; Terpineol; Terpinolene; Valencene; (−)-Terpinen-4-ol, and (−)-Terpinen-4-ol,
(b) the synthetic formulation or non-natural formulation comprises at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Famesene, (6E)-; beta-Ocimene; beta-Pinene; beta-Thujene; Bomeol; Camphene; Camphor; Cannabidiol; Cannabigerobc Acid; Carvone, (−)-; Caryophyllene Oxide; Cedrol; cis-2-Pinanol; cis-beta-ocimene; cis-Nerobdol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isobomeol, (−)-; Isopulegol; Limonene; Linalool; Menthol; Myrcene; Nerol; Nerobdol; p-Cymene; Phytol; Pulegone; Sabinene; Sabinene Hydrate; Terpineol; Terpinolene; Valencene; (−)-Terpinen-4-ol, and (−)-Terpinen-4-ol,
(c) the only terpene or terpenoids in the synthetic formulation comprise at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Famesene, (6E)-; beta-Ocimene; beta-Pinene; beta-Thujene; Bomeol; Camphene; Camphor; Cannabidiol; Cannabigerobc Acid; Carvone, (−)-; Caryophyllene Oxide; Cedrol; cis-2-Pinanol; cis-beta-ocimene; cis-Nerobdol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isobomeol, (−)-; Isopulegol; Limonene; Linalool; Menthol; Myrcene; Nerol; Nerobdol; p-Cymene; Phytol; Pulegone; Sabinene; Sabinene Hydrate; Terpineol; Terpinolene; Valencene; (−)-Terpinen-4-ol, and (−)-Terpinen-4-ol,
(d) the synthetic formulation, other than a solvent, thinner, diluent, dilutant or excipient, consists of at least one, 2, 3, 4, 5, 6, 7, 8, 9, 10, 11, 12, 13, 14, 15, 16, 17, 18, 19 or 20 or more of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Famesene, (6E)-; beta-Ocimene; beta-Pinene; beta-Thujene; Bomeol; Camphene; Camphor; Cannabidiol; Cannabigerobc Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isobomeol, (−)-; Isopulegol; Limonene; Linalool; Menthol; Myrcene; Nerol; Nerolidol; p-Cymene; Phytol; Pulegone; Sabinene; Sabinene Hydrate; Terpineol; Terpinolene; Valencene; (−)-Terpinen-4-ol, and (−)-Terpinen-4-ol,
(e) the synthetic or non-natural formulation comprises a formulation of any one of Formula #1 to #164, or
(f) any combination of (a) to (e).

2. The synthetic or non-natural formulation of claim 1, wherein:
at least one, 2, 3, 4, 5, 6, 7, 8, 9, 10, 11, 12, 13, 14, 15, 16, 17, 18, 19 or 20 or more of the terpene or terpenoid species is a synthetic terpene or terpenoid, or the synthetic formulation further comprises a non-terpene or non-terpenoid plant-derived phytochemical,
wherein optionally the non-terpene or non-terpenoid plant-derived phytochemical comprises a phytocannabinoid, a flavonoid, a cuticle wax or a plant-derived hydrocarbon,
wherein optionally the non-terpene or non-terpenoid plant-derived phytochemical, or the phytocannabinoid, comprises: tetrahydrocannabinol (THC), tetrahydrocannabivarin (THCV), cannabichromene (CBC), cannabichromanon (CBCN), cannabidiol (CBD), cannabielsoin (CBE), cannabidivarin (CBDV), cannbifuran (CBF), cannabicyclol (CBL), Cannabinol (CBN), Cannabinodiol (CBND), cannabitriol (CBT), cannabivarin (CBV), an isocanabinoid, cannabidiolic acid (CBDA), A9-tetrahydrocannabinol (A9-THC) or A8-tetrahydrocannabinol (D8-THC),
wherein optionally the non-terpene or non-terpenoid plant-derived phytochemical, or the phytocannabinoid, comprises: cannabigerolic Acid (CBGA), Cannabigerolic Acid, monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDV A), Cannabidivarin (CBDV), Cannabidiorcol (CBD-C1), Tetrahydrocannabinolic acid A (THC A-A), Tetrahydrocannabinolic acid B (THCA-B), Tetrahydrocannabinol (THC), Tetrahydrocannabinolic acid C4 (THCA-C4), Tetrahydrocannbinol C4 (THC-C4), Tetrahydrocannabivarinic acid (THCVA), Tetrahydrocannabivarin (THCV), Tetrahydrocannabiorcolic acid (THCA-C1), Tetrahydrocannabiorcol (THC-Ci), Delta-7-cis-iso-tetrahydrocannabivarin, A8-tetrahydrocannabinolic acid (A8-THCA), A8˜tetrahydrocannabinol (A8-TF1C), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinobc acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C₁), Cannabinodiol (CBND), Cannabinodivarin (CBDV), Cannabitriol (CBT), 10-Ethoxy-9-hydroxy-A6a-tetrahydrocannabinoi, 8, 9-Dihydroxy-A6a(10a)-tetrahydrocannabinol (8,9-Di-OH-CBT-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-A6a(10a) -tetrahydrocannabinol (OTHC), A9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Isocanabinoids, Epigallocatechin gallate, Dodeca-2E, 4E, 8Z, 10Z-tetraenoic acid isobutylamide, or Dodeca-2E,4E-dienoic acid isobutylamide,
wherein optionally the terpene, terpenoid or flavonoid is or comprises d-bmonene bnalool, 1,8-cineole (eucalyptol), α-pinene, terpineol-4-ol, p-cymene, bomeol, D-3-carene, b-sitosterol, b-myrcene, b-caryophyllene, cannflavin A, apigenin, quercetin or pulegone or any combination thereof,
wherein optionally the terpene, terpenoid or flavonoid is or comprises: alpha-bisabolol; bomeol; camphene; camphor; delta-3-carene; beta-caryophyllene; caryophyllene oxide; alpha-cedrene; beta-eudesmol; fenchyl alcohol; geraniol; guaiol; alpha-humulene; isobomeol; limonene; bnalool; menthol; myrcene; nerol; ocimene; trans-ocimene; alpha-phellandrene; alpha-pinene; beta-pinene; sabinene; alpha-terpinene; alpha-terpineol; terpinolene; alpha-guaiene; elemene; famesene; germacrene B; guaia-I(IO), 11-diene; trans-2-pinanol; selina-3, 7(1 I)-diene; eudesm-7(1 I)-en-4-ol; and/or valencene,
wherein optionally the terpene, terpenoid or flavonoid is or comprises: 7,8-dihydroionone, Acetanisole, Acetic Acid, Acetyl Cedrene, Anethole, Anisole, Benzaldehyde, Bergamotene (a-cis-Bergamotene) (a-trans-Bergamotene), Bisabolol (b-Bisabolol), Bomeol, Butanoic/Butyric Acid, Cadinene (a-Cadinene) (g-Cadinene), Cafestol, Caffeic acid, Camphene, Camphor, Capsaicin, Carene (D-3-Carene), Carotene, Carvacrol, Carvone, Dextro-Carvone, Laevo-Carvone, Caryophyllene (b-Caryophyllene), Caryophyllene oxide, Castoreum Absolute, Cedrene (α-Cedrene) (b-Cedrene), Cedrene Epoxide (α-Cedrene Epoxide), Cedrol, Cembrene, Chlorogenic Acid, Cinnamaldehyde (a-amyl-Cinnamaldehyde) (a-hexyl-Cinnamaldehyde), Cinnamic Acid, Cinnamyl Alcohol, Citronellal, Citronellol, Cryptone, Curcumene (a-Curcumene) (g-Curcumene), Decanal, Dehydrovomifobol, Diallyl Disulfide, Dihydroactinidiobde, Dimethyl Disulfide, Eicosane/lcosane, Elemene (b-Elemene), Estragole, Ethyl acetate, Ethyl Cinnamate, Ethyl maltol, Eucalyptol/1,8-Cineole, Eudesmol (a-Eudesmol) (b-Eudesmol) (g-Eudesmol), Eugenol, Euphol, Famesene, Famesol, Fenchol (b-Fenchol), Fenchone, Geraniol, Geranyl acetate, Germacrenes, Germacrene B, Guaia-1 (10),1 1-diene, Guaiacol, Guaiene (a-Guaiene), Gurjunene (a-Gurjunene), Hemiarin, Hexanaldehyde, Hexanoic Acid, Humulene (α-Humulene) (b-Humulene), lonol (3-oxo-a-ionol) (bïbhoï), Ionone (a-Ionone) (b-Ionone), Ipsdienol, Isoamyl acetate, Isoamyl Alcohol, Isoamyl Formate, Isobomeol, Isomyrcenol, Isopulegol, Isovaleric Acid, Isoprene, Kahweol, Lavandulol, Limonene, g-Linolenic Acid, Linalool, Longifolene, α-Longipinene, Lycopene, Menthol, Methyl butyrate, 3-Mercapto-2-Methylpentanal, Mercaptan/Thiols, b-Mercaptoethanol, Mercaptoacetic Acid, AIM Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (b-Myrcene), g-Muurolene, Nepetalactone, Nerol, Nerolidol, Neryl acetate, Nonanaldehyde, Nonanoic Acid, Ocimene, Octanal, Octanoic Acid, p-cymene, pentyl butyrate, phellandrene, phenylacetaldehyde, phenylethanethiol, Phenylacetic Acid, Phytol, Pinene, b-Pinene, propanethiol, Pristimerin, Pulegone, Retinol, Rutin, Sabinene, Sabinene Hydrate, cis-Sabinene Hydrate, trans-Sabinene Hydrate, Safranal, a-Selinene, a-Sinensal, b-Sinensal, b-Sitosterol, Squalene, Taxadiene, Terpin hydrate, Terpineol, Terpine-4-ol, a-Terpinene, g-Terpinene, Terpinolene, Thiophenol, Thujone, Thymol, a-Tocopherol, Tonka Undecanone, Undecanal, Valeraldehyde/Pentanal, Verdoxan, a-Ylangene, Umbelliferone or Vanillin,
wherein optionally the terpene, terpenoid or flavonoid is or comprises: a phenolic acid, a stilbenoid, a dihydroflavonol, an anthocyanin, an anthocyanidin, a polyphenol, a tannin, a flavone, flavan-3-ol, flavan-4-ol, flavan-3,4-diol flavonol, a stilbenoid, a phytochemicals, an antioxidant, a homoisoflavonoid, a phenylpropanoid, a phloroglucinol coumarin, a phenolic acid, a naphthodianthrone, a steroid glycoside, a bioflavonoid, an isoflavonoid, a neoflavonoid, adenosine, Adhyperforin, amentoflavone, Anandamide, Apigenin, Cannaflavin B, Catechin (C), Catechin 3-gallate (Cg), Chlorogenic acid, cichoric acid, caftaric acid, Daidzein, Delphinidin, Eleutherosides, epicatechin 3-gallate (ECg), Epicatechins, Epicatechin, epigallocatechin, myricetin, Oxalic acid, Pelargonidin, Tannin, Theaflavin-3-gallate, Theanine, Theobromine, Theophylline, Tryptophan, Tyramine, Xanthine, Caffeine, Cannaflavin A, Cannaflavin B, Catechin (C), Catechin 3-gallate (Cg), Epicatechin 3-gallate (ECg), Epicatechins (Epicatechin (EC)), epigallocatechin, Epigallocatechin (EGC), Epigallocatechin 3-gallate (EGCg), Gallocatechin (GC), Gallocatechin 3-gallate (GCg)), Gamma amino butyric acid, Genistein, Ginkgo biloba, Ginsenosides, Quercetin, Quercitrin or Rutin.

3. The synthetic or non-natural formulation of claim 1, further comprising a synthetic compound or a non-plant derived compound,
wherein optionally the synthetic compound comprises a synthetic: cannabinoid, a terpenoid, a flavonoid, a cuticle wax or a synthetic version of a plant-derived hydrocarbon, or a synthetic d-limonene linalool, 1,8-cineole (eucalyptol), a-pinene, terpineol-4-ol, p-cymene, bomeol, D-3-carene, b-sitosterol, b-myrcene, b-caryophyllene, cannflavin A, apigenin, quercetin or pulegone, or a synthetic version of a compound as listed in Table 1.

4. The synthetic or non-natural formulation of claim 1, wherein the synthetic formulation further comprises or is formulated with a solvent, thinner, or a diluent (or dilutant),
and optionally the solvent, thinner, or a diluent comprises squalane, squalene, 1,3-propanediol, acetone, acetonitrile, an alcohol, triacetin, dimethyl sulfoxide, dimethylformamide, phenol, propylene glycol, dipropylene glycol, 1,3-propanediol, isophytol, and/or phytol,
and optionally the alcohol is or comprises a benzyl alcohol, or ethanol, butanol, 2-ethylhexanol, isobutanol, isopropanol, methanol, propanol or propylene glycol.

5. The synthetic or non-natural formulation of claim 1, wherein the synthetic formulation further comprises or is formulated with a stabilizer, anti-oxidant or a compound to prevent crystallization,
wherein optionally the stabilizer, anti-oxidant or a compound to prevent crystallization comprises triethyl citrate.

6. The synthetic or non-natural formulation of claim 1, wherein the synthetic formulation comprises or further comprises a non-plant derived compound or synthetic compound,
and optionally the non-plant derived compound or synthetic compound comprises an organic compound having a odor detectable to a human, wherein optionally the organic compound having a odor detectable to a human comprise or is:
3-methyl-2-butene-1-thiol; hexanoic acid hexyl ester; 2-heptanone, octanal or cis-4-decenal,
and optionally the non-plant derived compound or synthetic compound comprises an organic compound having a taste detectable to a human, wherein optionally the organic compound having a taste detectable to a human comprise or is:
4-hydroxy-2,5-dimethyl-3(2H)-furanone (strawberry), ethyl butyrate (apple, fruity), isoamyl acetate (banana), propyl hexanoate (pineapple, fruity), allyl hexanoate (pineapple, fruity), valencene (orange, fresh fruity), methyl anthranilate (also known as methyl 2-aminobenzoate) (grape), methyl butyrate (fruity, apple, pineapple), benzyl acetate (fruity, strawberry), p-mentha-8-thiol-3-one (grapefruit), (IS,4S)-trans-p-menthan-8-thiol-3-one acetate (black currant, exotic), or (IR,4S)-cis-p-menthan-8-thiol-3-one acetate (fruity, sweet),
and optionally the non-plant derived compound or synthetic comprises a surfactant, and optionally the surfactant comprises or is polysorbate 20, a TWEEN, a stearate, ammonium lauryl sulfate, sodium lauryl sulfate, perfluorooctanesulfonate (PFOS), dioctyl sodium sulfosuccinate, a siloxane, cetrimonium bromide (CTAB), cetylpyridinium chloride (CPC), benzalkonium chloride (BAC), benzethonium chloride (BZT), dimethyldioctadecylammonium chloride, octaethylene glycol monododecyl ether, pentaethylene glycol monododecyl ether and dioctadecyldimethylammonium bromide (DODAB); or the surfactants is an anionic, cationic, nonionic or zwitterionic surfactant.

7. The synthetic or non-natural formulation of claim 1, comprising or further comprising: cannabigerolic Acid (CBGA), Cannabigerolic Acid monomethyl ether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiobc Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-Ci), Tetrahydrocannabinobc acid A (THCA-A), tetrahydrocannabinobc acid B (THCA-B), Tetrahydrocannabinol (THC), Tetrahydrocannabinobc acid C4 (THCA-C4), Tetrahydrocannbinol C4 (THC-C4), Tetrahydrocannabivarinic acid (THCVA), Tetrahydrocannabivarin (THCV), Tetrahydrocannabiorcobc acid (THCA-Ci), Tetrahydrocannabiorcol (THC-Ci), Delta-7-cis-iso-tetrahydrocannabivarin, A8-tetrahydrocannabinobc acid (A8-THC A), D8-tetrahydrocannabinol (A8-THC), Cannabicyclobc acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinobc acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-Ci), Cannabinodiol (CBND), Cannabinodivarin (CBDV), Cannabitriol (CBT), 10-Ethoxy-9-hydroxy-A6a-tetrahydrocannabinol, 8,9-Dihydroxy-A6a(10a)˜tetrahydrocannabinol (8,9-Di-OH-CBT-Cs), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oco-D'af I,Ia) t-etrahydrocannabinol (OTHC), As-c/s˜tetrahydrocannabinol (cis-THC), Cannabiripsol (CBR), 3,4,5,6-tetrahydro-7-hydroxy-alpha-alpha-2-trimethyl-9-n-propyl-2,6-methano-2H-I-benzoxocin-5-methanol (OH-iso-HHCV), Trihydroxy-delta-9-tetrahydrocannabinol (triOH-THC), an isocanabinoid or epigallocatechin gallate.

8. The synthetic or non-natural formulation of claim 1, further comprising a mineral, wherein optionally the mineral comprises or is lithium.

9. The synthetic or non-natural formulation of claim 1, where the synthetic formulation comprises:
(a) one, two, three, 4, 5, 6, 7, 8, 9, 10 or all of the following terepenes: 3-Carene; 3,7(11)-Eudesmadiene; 4-Carvomenthenol; 4-Thujanol; alpha-Bergamotene; alpha-Bisabolol, (+)-; alpha-Bulnesene; alpha-Cedrene; alpha-Guaiene; alpha-Ocimene, (3E)-; alpha-Phellandrene; alpha-Pinene; alpha-Terpinene; Aromadendrene; beta-Caryophyllene; beta-Elemene; beta-Famesene, (6E)-; beta-Ocimene; beta-Pinene; beta-Thujene; Bomeol; Camphene; Camphor; Cannabidiol; Cannabigerolic Acid; Carvone, (−)-; Caryophyllene Oxide; Cedrol; cis-2-Pinanol; cis-beta-ocimene; cis-Nerolidol; Citronellol; d-Limonene; delta8-THC; Dronabinol; Eucalyptol; Fenchone; Fenchol; gamma-Elemene; gamma-Terpinene; Geraniol; Geranyl Acetate; Germacrene B; Guaiol; Humulene; Isobomeol, (−)-; Isopulegol; Limonene; Linalool; Menthol; Myrcene; Nerol; Nerobdol; p-Cymene; Phytol; Pulegone; Sabinene; Sabinene Hydrate; Terpineol; Terpinolene; Valencene; (−)-Terpinen-4-ol, and (−)-Terpinen-4-ol;
(b) the synthetic formulation of (a), wherein the only terpenes in the synthetic formulation are one, two, three, 4, 5, 6, 7, 8, 9, 10 or all of the terpenes listed in (a); or
(c) the synthetic formulation of (a), wherein the terpenes are present in approximately equal amounts.

10. A product of manufacture comprising or having contained therein a synthetic or non-natural formulation of claim 1,
wherein optionally the product of manufacture is a medical device, a patch, a implant or a dispenser (optionally a nebulizer or an aerosol dispenser).

11. A pharmaceutical or nutraceutical formation comprising a synthetic or non-natural formulation of claim 1,
wherein optionally the pharmaceutical or nutraceutical formulation is manufactured or formulated for oral, sublingual, buccal, aerosol or topical administration,
wherein optionally the pharmaceutical or nutraceutical formulation is manufactured or formulated as a liquid, a tincture, an aerosol, a mist, a gel, a tablet, a capsule, a gel, a geltab, a pill or a powder,
wherein optionally the pharmaceutical or nutraceutical formulation further comprises a pharmaceutically acceptable excipient, or a sweetener, wherein the sweetener is a Stevia extract.

12. A method for treating, preventing or ameliorating a cancer or an epileptic seizure in an individual in need thereof, or for generating a sedative or energizing effect in an individual in need thereof, comprising administering a pharmaceutical or nutraceutical formulation comprising a synthetic or non-natural formulation of claim 1.

13. (canceled)

14. A method for treating, preventing or ameliorating anxiety in an individual in need thereof comprising administering a pharmaceutical or nutraceutical formulation comprising a synthetic or non-natural formulation of claim 1.

15. A method for treating, preventing or ameliorating spasms in an individual in need thereof comprising administering a pharmaceutical or nutraceutical formulation comprising a synthetic or non-natural formulation of claim 1.

16. A method for immunosupressing an individual in need thereof comprising administering a pharmaceutical or nutraceutical formulation comprising a synthetic or non-natural formulation of claim 1.

17. A method for suppressing appetite in an individual in need thereof comprising administering a pharmaceutical or nutraceutical formulation comprising a synthetic or non-natural formulation of claim 1.

18. A method for dilating bronchi in an individual in need thereof comprising administering a pharmaceutical or nutraceutical formulation comprising a synthetic or non-natural formulation of claim 1.
